(19) United States
(12) Patent Application Publication
(10) Pub. No.: US 2022/0054528 A1
(43) Pub. Date: Feb. 24, 2022

(54) TREATMENT OF ADULT T CELL LEUKEMIA/LYMPHOMA (ATLL) USING MIR-155 INHIBITORS

(71) Applicant: MIRAGEN THERAPEUTICS, INC., Boulder, CO (US)

(72) Inventors: Diana M. ESCOLAR, Boulder, CO (US); Linda Anne PESTANO, Boulder, CO (US); Paul D. RUBIN, Boulder, CO (US)

(21) Appl. No.: 17/299,415

(22) PCT Filed: May 15, 2019
(86) PCT No.: PCT/US19/32424

§ 371 Date: Jun. 3, 2021

Related U.S. Application Data
(60) Provisional application No. 62/774,805, filed on Dec. 3, 2018.

(51) Int. Cl. A61K 31/7125; A61P 35/00; A61K 31/7072; A61K 38/21; A61K 31/454; A61K 31/7048; A61K 31/573; A61K 31/475; A61K 31/675; A61K 31/704; A61K 38/15; A61K 31/522

(52) U.S. Cl. CPC A61K 31/7125; A61P 35/00; A61K 31/7072; A61K 38/212; A61K 31/454; A61K 31/522; A61K 31/573; A61K 31/475; A61K 31/675; A61K 31/704; A61K 38/15; A61K 31/7048

(57) ABSTRACT

The present disclosure provides methods for treating Adult T-cell Leukemia/Lymphoma (ATLL) in a subject by administering to the subject an oligonucleotide inhibitor of miR-155. In some embodiments, the subject may have received one or more therapies for treating ATLL prior to receiving the oligonucleotide inhibitor of miR-155. Administration of an oligonucleotide inhibitor of miR-155 according to the methods of the present disclosure can decrease the tumor cell count or can maintain the tumor cell count at or below the tumor cell count at the beginning of administration of the oligonucleotide inhibitor of miR-155. Accordingly, the methods provided in this disclosure can be used as maintenance therapies for treating ATLL subjects.

### CROSS-REFERENCE TO RELATED APPLICATIONS

The present application claims the benefit of priority to U.S. Provisional Application No. 62/774,805, filed on Dec. 3, 2018, the contents of which are hereby incorporated by reference in their entireties.

### INCORPORATION BY REFERENCE OF THE SEQUENCE LISTING

The contents of the text file submitted electronically herewith are incorporated herein by reference in their entirety: A computer readable format copy of the Sequence Listing (filename: MIRG_063_01WO_SeqList_ST25.txt, date recorded May 8, 2019, file size 106,948 bytes).

### FIELD OF THE DISCLOSURE

The present invention relates to methods for treating Adult T-cell Leukemia/Lymphoma (ATLL), methods for decreasing or maintaining the tumor cell count in a subject suffering from ATLL, and/or methods for treating a disease associated with a Human T-cell Lymphotropic Virus (HTLV), wherein the methods comprise administering an oligonucleotide inhibitor of miR-155.

### BACKGROUND

Adult T-cell leukaemia/lymphoma (ATLL) is a T-lymphoproliferative disorder of mature helper/inducer T-cell origin (CD4+, CD29+, CD45R−) and is etiologically linked to the human T-cell lymphotropic virus, in particular, HTLV-I. The disease manifests with leukaemia in greater than two thirds of patients, while the remaining patients have a lymphomatous form.

The clinical course of ATLL is aggressive with a median survival ranging from a few days or weeks to at best, a few months in the acute and lymphomatous forms. Despite major advances in understanding the pathogenesis of the disease, management of these patients remains a challenge for clinicians as they do not respond or achieve only transient responses to therapies used in high-grade lymphomas. Thus, there remains an unmet medical need for new therapies to treat ATLL.

### SUMMARY OF THE INVENTION

In some embodiments, provided herein is a method for treating ATLL in a subject in need thereof, the method comprising (a) administering to the subject one or more therapeutic agents for treating ATLL; and (b) administering an oligonucleotide inhibitor of miR-155 to the subject after the administration of the one or more therapeutic agents.

In some embodiments, provided herein is a method for decreasing or maintaining the tumor cell count in a subject suffering from ATLL, the method comprising administering an oligonucleotide inhibitor of miR-155 to the subject, wherein the subject has received one or more therapeutic agents for treating ATLL prior to the treatment with the oligonucleotide inhibitor of miR-155.

In some embodiments, provided herein is a method for treating ATLL in a subject in need thereof, comprising administering to the subject an oligonucleotide inhibitor of miR-155, wherein the oligonucleotide inhibitor of miR-155 decreases or maintains the tumor cell count in the subject.

Also provided herein are methods for treating a disease associated with a Human T-cell Lymphotropic Virus (HTLV) in a subject in need thereof, comprising administering to the subject an oligonucleotide inhibitor of miR-155. The disease associated with HTLV can be caused by HTLV-1, HTLV-2, HTLV-3, or HTLV-4. In some embodiments, a disease associated with HTLV is ATLL, HTLV-1 associated myelopathy/tropical spastic paraparesis (HAM/TSP), or HTLV-associated dermatitis.

ATLL treated by the method of the present disclosure can be acute ATLL, lymphomatous ATLL, smouldering ATLL or chronic ATLL. In some embodiments, acute ATLL is acute leukemic ATLL. In some embodiments, lymphomatous ATLL is acute lymphomatous ATLL.

In some embodiments, the subject suffering from ATLL (may also referred to as “ATLL subject”) has received one or more therapeutic agents for treating ATLL prior to administration of the oligonucleotide inhibitor of miR-155.

In some embodiments, the subject suffering from ATLL (may also referred to as “ATLL subject”) may receive one or more therapeutic agents for treating ATLL after or concurrently with administration of the oligonucleotide inhibitor of miR-155.

The one or more therapeutic agents that may be administered to the ATLL subject prior to, after, or concurrently with administration of an oligonucleotide inhibitor of miR-155 include, but are not limited to, zidovudine, interferon alpha, lenalidomide, etoposide, prednisone, vincristine, cyclophosphamide, doxorubicin hydrochloride, rituximab, romidepsin, mogamulizumab, pralatrexate, triamcinolone acetonide, ranimustine, vindesine, carboplatin, ALRN-6924 (a stapled alpha-helical peptide), methotrexate, gemcitabine, oxaliplatin, belinostat, acyclovir, Histone deacetylase (HDAC) inhibitors (HDIs) such as valproic acid, radiation therapy, and/or combinations thereof.

In some embodiments, one or more therapeutic agents that may be administered to the ATLL subject prior to, after, or concurrently with the administration of an oligonucleotide inhibitor of miR-155 are zidovudine, interferon alpha, lenalidomide, etoposide, prednisone, vincristine, cyclophosphamide, and doxorubicin hydrochloride.

In some embodiments, one or more therapeutic agents that may be administered to the ATLL subject prior to, after, or concurrently with the administration of an oligonucleotide inhibitor of miR-155 cyclophosphamide, doxorubicin, vincristine, etoposide, and prednisone.

In some embodiments, one or more therapeutic agents that may be administered to the ATLL subject prior to, after, or concurrently with the administration of an oligonucleotide inhibitor of miR-155 are etoposide, zidovudine, interferon alpha, romidepsin, mogamulizumab, pralatrexate, lenalidomide, triamcinolone acetonide, and ALRN-6924.

In some embodiments, one or more therapeutic agents that may be administered to the ATLL subject prior to, after, or concurrently with the administration of an oligonucleotide inhibitor of miR-155 include VCAP-AMP-VECP chemotherapy (vincristine, cyclophosphamide, doxorubicin, and prednisone (VCAP), doxorubicin, ranimustine, and prednisone (AMP), and vindesine, etoposide, carboplatin, and prednisone (VECP)), zidovudine and interferon alpha (AZT/IFN), gemcitabine with oxaliplatin, belinostat, pralatrexate, IT chemotherapy and zidovudine with valproic acid (AZT/VPA).

In some embodiments, the ATLL subject may receive a phototherapy or a photochemotherapy prior to, after, or concurrently with the administration of an oligonucleotide inhibitor of miR-155. In some embodiments, the photochemotherapy is a combination treatment comprising psoralens and long wave ultraviolet radiation.

In some embodiments, the ATLL subject may receive a radiation therapy prior to, after, or concurrently with the administration of an oligonucleotide inhibitor of miR-155.

In some embodiments, the one or more therapeutic agents for treating ATLL are administered to the subject at least for one cycle prior to the administration of the oligonucleotide inhibitor of miR-155.

In any one of the methods of the present disclosure, an oligonucleotide inhibitor of miR-155 can be administered according to a cycle, said cycle comprising: (a) first administering at least one loading dose; and (b) second administering at least one maintenance dose.

In some embodiments, the loading dose can be about 2 times, 3 times, 4 times, or 5 times the maintenance dose.

In some embodiments, the loading dose can range from about 1500 mg to about 2400 mg. In some embodiments, the loading dose is about 1500 mg, 1800 mg, 2100 mg, or about 2400 mg. In an exemplary embodiment, the loading dose can be about 1800 mg.

In some embodiments, the loading dose can be administered as a bolus dose. In some embodiments, the loading dose can be split into equal doses administered on consecutive days or alternative days over a period of 7 to 10 days. In some embodiments, the loading dose can be split into three doses administered every day for three consecutive days. In some embodiments, the loading dose can be split into three doses administered every other day. In an exemplary embodiment, the loading dose can be about 1800 mg split into three doses with each split dose being about 600 mg administered every other day. In another exemplary embodiment, the loading dose can be about 1800 mg split into three doses with each split dose being about 600 mg administered on three consecutive days. In another exemplary embodiment, the loading dose can be about 1800 mg split into three doses of about 600 mg each administered on day 1, 3, and 5 of the cycle. In another exemplary embodiment, the loading dose can be about 1800 mg split into three doses of about 600 mg each administered on day 1, 2, and 3 of the cycle.

In some embodiments, the maintenance dose may range from about 400 mg to about 1200 mg. In some embodiments, the maintenance dose can be about 400 mg, 600 mg, 800 mg, 1000 mg, or about 1200 mg. In some embodiments, the maintenance dose is about 600 mg.

In some embodiments, the maintenance dose is administered once a week. In some embodiments, the maintenance dose is administered twice a week. In an exemplary embodiment, the maintenance dose is about 600 mg administered once a week.

In some embodiments, an oligonucleotide inhibitor of miR-155 that may be administered in any one of the methods of the present disclosure may comprise a sequence selected from Table 2.

In some embodiments, an oligonucleotide inhibitor of miR-155 that may be administered in any one of the methods of the present disclosure comprises the sequence of 5′-lCs.dAs.lCs.dGs.dAs.lTs.lTs.dAs.lGs.dCs.lAs.lTs.lTs.lA-3′ (SEQ ID NO: 25).

In some embodiments, administration of an oligonucleotide inhibitor of miR-155 decreases the tumor cell count in the ATLL subject compared to the tumor cell count at the beginning of the administration of the oligonucleotide inhibitor of miR-155.

In some embodiments, administration of an oligonucleotide inhibitor of miR-155 decreases the tumor cell count in the ATLL subject compared to the tumor cell count at the beginning of the administration of the oligonucleotide inhibitor of miR-155 and maintains the tumor cell count at or below the decreased level.

In some embodiments, administration of an oligonucleotide inhibitor of miR-155 decreases the tumor cell count compared to the tumor cell count at the beginning of the administration of the oligonucleotide inhibitor of miR-155 and maintains the tumor cell count to the decreased level for 6 months or more.

In some embodiments, administration of an oligonucleotide inhibitor of miR-155 maintains the tumor cell count to the level that was present at the beginning of the administration of the oligonucleotide inhibitor of miR-155 for 6 months or more.

In some embodiments, administration of an oligonucleotide inhibitor of miR-155 may decrease the tumor cell count in the ATLL subject by about 10 to 80% compared to the tumor cell count at the beginning of the administration of the oligonucleotide inhibitor of miR-155.

In some embodiments, administration of an oligonucleotide inhibitor of miR-155 maintains the tumor cell count at or below the level that was present at the beginning of the administration of the oligonucleotide inhibitor of miR-155.

In some embodiments, administration of an oligonucleotide inhibitor of miR-155 decreases the size of enlarged lymph nodes in the ATLL subject.

In some embodiments, administration of an oligonucleotide inhibitor of miR-155 decreases the size of enlarged lymph nodes in the ATLL subject to a normal size.

In some embodiments, one or more therapeutic agents administered to the ATLL subject prior to the administration of an oligonucleotide inhibitor of miR-155 may decrease the size of lymph nodes in the subject and subsequent administration of an oligonucleotide inhibitor of miR-155 to the subject may maintain the size of lymph nodes to the decreased size.

In some embodiments, administration of an oligonucleotide inhibitor of miR-155 to an ATLL subject decreases the activation and/or proliferation of tumor cells.

In some embodiments, administration of an oligonucleotide inhibitor of miR-155 to an ATLL subject decreases the activation of tumor cells as measured by the levels of activation markers HLA-DR and/or CD69.

In some embodiments, administration of an oligonucleotide inhibitor of miR-155 to an ATLL subject may decrease the levels of activation markers HLA-DR and/or CD69 on tumor cells by about 5-80% compared to the levels at the beginning of the administration of the oligonucleotide inhibitor of miR-155.

In some embodiments, administration of an oligonucleotide inhibitor of miR-155 to an ATLL subject decreases the proliferation of tumor cells as measured by the levels of cell cycle marker Ki67.

In some embodiments, administration of an oligonucleotide inhibitor of miR-155 to an ATLL subject may decrease the levels of cell cycle marker Ki67 on tumor cells by about 5-80% compared to the levels at the beginning of the administration of the oligonucleotide inhibitor of miR-155.

In some embodiments, administration of an oligonucleotide inhibitor of miR-155 to an ATLL subject may decrease the proliferation of tumor cells by about 5-90% compared to the proliferation of tumor cells at the beginning of the administration of the oligonucleotide inhibitor of miR-155.

In some embodiments, an oligonucleotide inhibitor of miR-155 may be administered to an ATLL subject or a subject having a disease associated with HTLV for about 6 weeks, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 2.5 year, 3 years, 3.5 years, 4 years, 4.5 years, 5 years, 5.5 years, 6 years, 6.5 years, 7 years, 7.5 years, 8 years, 8.5 years, 9 years, 9.5 years, 10 years or more.

In some embodiments, administration of an oligonucleotide inhibitor of miR-155 increases the progression-free survival of the subject by at least about 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 2.5 year, 3 years, 3.5 years, 4 years, 4.5 years, 5 years, 5.5 years, 6 years, 6.5 years, 7 years, 7.5 years, 8 years, 8.5 years, 9 years, 9.5 years, 10 years or more.

In some embodiments, administration of an oligonucleotide inhibitor of miR-155 reduces the HTLV load in the ATLL subject or in the subject having a disease associated with HTTLV. In some embodiments, administration of an oligonucleotide inhibitor of miR-155 reduces the HTLV load in the subject by about 10-80%, compared to the HTLV load at the beginning of administration of the oligonucleotide inhibitor of miR-155.

In some embodiments, administration of an oligonucleotide inhibitor of miR-155 to a subject promotes restoration of normal homeostatic immune response in the subject. For example, in some embodiments, administration of an oligonucleotide inhibitor of miR-155 promotes re-population of healthy B and/or T cells in the subject. In some embodiments, administration of an oligonucleotide inhibitor of miR-155 to a subject increases the number of healthy B and/or T cells in the subject by about 5 to 60%.

### BRIEF DESCRIPTION OF THE DRAWINGS

FIG. 1A shows the cell count of white blood cells (WBCs) and malignant T cells in Patient No. 101-008 with acute leukemic ATLL from the beginning of the ATLL treatment until filing of the provisional application.

FIG. 1B shows the cell count of white blood cells (WBCs) and malignant T cells in Patient No. 101-008 with acute leukemic ATLL from the beginning of the ATLL treatment until filing of the present application.

FIG. 2A shows the status of activation markers HLA-DR (upper panel) and CD69 (lower panel), expressed as mean fluorescent intensity (MFI) and percent positive tumor cells, over 6 cycles of antimiR-155 treatment (from the beginning of the antimiR-155 treatment until filing of the provisional application) in Patient No. 101-008. C1D1 stands for Cycle 1 Day 1, C1D5 stands for Cycle 1 Day 5 and so forth.

FIG. 2B shows the status of a proliferation marker, Ki67 (upper panel), and an apoptosis marker, cPARP (lower panel), expressed as percent positive tumor cells, over 6 cycles of antimiR-155 treatment (from the beginning of the antimiR-155 treatment until filing of the provisional application) in Patient No. 101-008.

FIG. 2C shows percentage of tumor cells in Patient No. 101-008 from the beginning of the ATLL treatment until the filing of the provisional application.

FIG. 2D shows percentage of tumor cells in Patient No. 101-008 from the beginning of the ATLL treatment until the filing of the present application.

FIG. 2E shows the status of activation markers HLA-DR (upper panel) and CD69 (middle panel), and the status of a proliferation marker, Ki67 (lower panel), expressed as mean fluorescent intensity (MFI) and percent positive tumor cells, from the beginning of the antimiR-155 treatment until filing of the present application, in Patient No. 101-008.

FIG. 2F shows the percentage of ATL cells positive for each biomarker over the course of the antimiR-155 treatment.

FIG. 2G shows the fold change from baseline (C1D1) in the levels of the biomarkers over the course of the antimiR-155 treatment.

FIG. 3A shows the levels of hematocrit and hemoglobin and the RBC count in Patient No. 101-008 from the beginning of the antimiR-155 treatment until filing of the provisional application.

FIG. 3B shows the cell count of CD8⁺ T cells, Natural Killer (NK) cells, and B cells in Patient No. 101-008 from the beginning of the antimiR-155 treatment until filing of the provisional application.

FIG. 3C shows the appearance of B cells developing over time (upper panel), further characterization of those B cells as non-plasmablasts vs plasmablasts (middle panel), and maturing naïve B cells (lower panel) that are within the non-plasmablast gate in the peripheral blood from Patient No. 101-008 over the course of antimiR-155 treatment.

FIG. 3D shows the levels of hematocrit and hemoglobin and the RBC count in Patient No. 101-008 from the beginning of the antimiR-155 treatment until filing of the present application.

FIG. 3E shows the cell count of CD8⁺ T cells, Natural Killer (NK) cells, and B cells in Patient No. 101-008 from the beginning of the antimiR-155 treatment until filing of the present application.

FIG. 3F shows a graphical representation of B cell maturation as % mature B cell (CD10⁻CD27⁻IgD⁺) and % transitional B cells (CD10⁺CD38⁺⁺) of CD19⁺ cells over the course of the antimiR-155 treatment.

FIG. 4A shows the cell count of white blood cells (WBCs) and malignant T cells in Patient No. 101-010 with lymphomatous ATLL from the beginning of the ATLL treatment until filing of the provisional application. The spike in WBC count is attributed primarily to large increase in neutrophils and correlates with a reported rhinovirus infection.

FIG. 4B shows the cell count of white blood cells (WBCs) and malignant T cells in Patient No. 101-010 with lymphomatous ATLL from the beginning of the ATLL treatment until filing of the present application. The spike in WBC count is attributed primarily to large increase in neutrophils and correlates with a reported rhinovirus infection.

FIG. 5A shows the status of activation markers HLA-DR (upper panel) and CD69 (lower panel), expressed as mean fluorescent intensity (MFI) and percent positive tumor cells, over 4 cycles of antimiR-155 treatment (from the beginning of the antimiR-155 treatment until filing of the provisional application) in Patient No. 101-010. C1D1 stands for Cycle 1 Day 1, C1D5 stands for Cycle 1 Day 5 and so forth.

FIG. 5B shows the status of a proliferation marker, Ki67 (upper panel), and an apoptosis marker, cPARP (lower panel), expressed as percent positive tumor cells, over 4 cycles of antimiR-155 treatment (from the beginning of the antimiR-155 treatment until filing of the provisional application) in Patient No. 101-010.

FIG. 5C shows the percentage of tumor cells in Patient No. 101-010 from the beginning of the ATLL treatment until the filing of the provisional application.

FIG. 5D shows the percentage of tumor cells in Patient No. 101-010 from the beginning of the ATLL treatment until the filing of the present application.

FIG. 5E shows the status of activation markers HLA-DR (upper panel) and CD69 (lower panel), expressed as mean fluorescent intensity (MFI) and percent positive tumor cells, from the beginning of the antimiR-155 treatment until filing of the present application, in Patient No. 101-010.

FIG. 5F shows the status of a proliferation marker, Ki67 (upper panel), and an apoptosis marker, cPARP (lower panel), expressed as percent positive tumor cells, from the beginning of the antimiR-155 treatment until filing of the present application, in Patient No. 101-010.

FIG. 5G shows the percentage of ATL cells positive for each biomarker over the course of the antimiR-155 treatment.

FIG. 5H shows the fold change from baseline (C1D1) in the levels of the biomarkers over the course of the antimiR-155 treatment.

FIG. 6A shows the cell count of white blood cells (WBCs) and malignant T cells in Patient No. 102-012 with lymphomatous ATLL over 3 cycles of antimiR-155 treatment.

FIG. 6B shows the mSWAT score in Patient No. 102-012 over 3 cycles of antimiR-155 treatment and for 102-015 after the same patient re-enrolled on the study to continue treatment for an additional 7 doses.

FIG. 7A shows the status of activation markers HLA-DR (upper panel) and CD69 (lower panel), expressed as mean fluorescent intensity (MFI) and percent positive tumor cells, over 2 cycles of antimiR-155 treatment in Patient No. 102-012.

FIG. 7B shows the cell count of ATLL cells, CD69+ ATLL cells, and HLA-DR+ ATLL cells in Patient No. 102-012 over 2 cycles of antimiR-155 treatment.

FIG. 8A shows the status of a proliferation marker, Ki67 (upper panel), and an apoptosis marker, cPARP (lower panel), expressed as percent positive tumor cells, over 2 cycles of antimiR-155 treatment in Patient No. 102-012.

FIG. 8B is a graphical representation of the data in FIG. 8A showing percentage of tumor cells positive for Ki-67 and cPARP over 2 cycles of antimiR-155 treatment in Patient No. 102-012.

FIG. 9A shows the levels of lactate dehydrogenase (LDH) in Patient No. 101-011 with acute lymphomatous ATLL over one cycle of antimiR-155 treatment and the following round of chemotherapy.

FIG. 9B shows the cell count of neutrophils, lymphocytes, and WBCs in Patient No. 101-011 over one cycle of antimiR-155 treatment.

FIG. 9C shows the percentage of CD3+ T cell, CD4+ T cells, and CD4+CD7− tumor cells in Patient No. 101-011 over one cycle of antimiR-155 treatment.

FIG. 10A shows the cell count of neutrophils, lymphocytes, and WBCs in Patient No. 119-001 with acute leukemic ATLL over one cycle of antimiR-155 treatment.

FIG. 10B shows the levels of LDH in Patient No. 119-001 over one cycle of antimiR-155 treatment.

FIG. 10C shows the LDH levels in Patient No. 119-001 from the beginning of the antimiR-155 treatment until filing of the present application.

FIG. 10D shows a percent change in the size of 4 lymph nodes as assessed by the CT scan over the course of the antimiR-155 treatment.

FIG. 11A shows a graphical representation of the average fold change from baseline of the percent peripheral blood ATL tumor cells positive for activation markers CD69 and HLA-DR and proliferation marker Ki-67 for all evaluable patients at each time point.

FIG. 11B shows a graphical representation of the average fold change from baseline of the percent peripheral blood ATL tumor cells positive for activation markers CD69 and HLA-DR for all evaluable patients at the indicated time points. Number of patients averaged indicated for each cycle shown.

FIG. 11C shows a graphical representation of the average fold change from baseline of the percent peripheral blood ATL tumor cells positive for proliferation marker Ki-67 for all evaluable patients at the indicated time points. Number of patients averaged indicated for each cycle shown.

FIG. 12 shows the cell count of WBCs and malignant T cells in Patient No. 101-012 with acute ATLL from the beginning of the ATLL treatment until filing of the present application.

FIG. 13A shows the LDH levels over the course of the treatment from the patients that were in partial remission at the start of study.

FIG. 13B shows the LDH levels over the course of the treatment from the patients that came off the study.

### DETAILED DESCRIPTION

In the context of the present disclosure, the terms “oligonucleotide inhibitor”, “antimiR” (e.g., antimiR-155), “antisense oligonucleotide or ASO”, or “anti-microRNA oligonucleotide or AMO” are used broadly and encompass an oligomer comprising ribonucleotides, deoxyribonucleotides, modified ribonucleotides, modified deoxyribonucleotides or a combination thereof, that inhibits the activity or function of the target microRNA (miRNA) by fully or partially hybridizing to the miRNA thereby repressing the function or activity of the target miRNA.

The term “miR-155” as used herein includes pri-miR-155, pre-miR-155, miR-155-5p, and hsa-miR-155-5p.

The term “about” as used herein encompasses variations of +/−10% and more preferably +/−5%, as such variations are appropriate for practicing the present invention.

The term “subject” or “patient” as used herein refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In other embodiments, the subject is a human.

In humans, miR-155 is encoded by the MIR155 host gene or MIR155HG and is located on human chromosome 21. Since both arms of pre-miR-155 can give rise to mature miRNAs, processing products of pre-miR-155 are designated as miR-155-5p (from the 5′ arm) and miR-155-3p (from the 3′ arm). miR-155-5p is expressed in hematopoietic cells including B-cells, T-cells, monocytes and granulocytes. The mature sequences for human miR-155-5p and miR-155-3p are given below:

Human mature miR-155-5p
(SEQ ID NO: 1)
5′-UUAAUGCUAAUCGUGAUAGGGGU-3′
Human mature miR-155-3p
(SEQ ID NO: 2)
5′-CUCCUACAUAUUAGCAUUAACA-3′

The prognosis of ATLL is poor as most patients do not respond or have short-lived partial responses to treatments used in other T-cell lymphomas. In particular, it has been found that the current treatment options may not decrease the number of malignant T cells in the ATLL patients effectively thereby play a major role in poor prognosis. For example, in some patients, the current treatment options may decrease the tumor cell count; however, the decrease can be temporary and the tumor cells can increase in numbers once the treatment is over. In some patients, the current treatment options may not decrease the tumor cell count substantially. The present invention is based, in part, on a surprising finding by the inventors that the administration of an oligonucleotide inhibitor of miR-155 to ATLL patients decreases the tumor cell count or maintains the tumor cell count at or below the level that was present at the beginning of the administration of the oligonucleotide inhibitor of miR-155. Accordingly, in one aspect, the present disclosure provides a maintenance therapy for treating ATLL patients, wherein the maintenance therapy comprises administration of an oligonucleotide inhibitor of miR-155 to the ATLL patient to decrease the tumor cell count or maintain the tumor cell count at or below the level that was present at the beginning of the administration of the oligonucleotide inhibitor of miR-155. In some embodiments, an oligonucleotide inhibitor of miR-155 can be administered for extended period of time to maintain the tumor cell count at or below the level that was present at the beginning of the administration of the oligonucleotide inhibitor of miR-155.

According to the disease manifestations, there are four sub-types of ATLL: acute, chronic, smouldering, and lymphoma. These sub-types are summarized in the table below.

TABLE 1

| ATLL | |
|---|---|
| Sub-type | Clinical Symptoms |
| Acute | Aggressive. Leukaemic picture, organomegaly (larger than |
| | normal lymph nodes, liver, and spleen), high lactate |
| | dehydrogenase (LDH), often hypercalcaemia, rashes, raised |
| | patches or lumps on the skin, higher than normal white |
| | blood cell, lymphocyte and T cell counts. |
| Chronic | Less aggressive. It can affect the skin, lungs, liver or |
| | spleen and causes enlarged lymph nodes. Higher than |
| | normal numbers of lymphocytes and T cells in the blood. |
| Smouldering | Less aggressive. It can affect the skin or the lungs. It |
| | causes abnormal T cell counts, but the number of |
| | lymphocytes in the blood is not higher than normal. |
| Lympho- | Aggressive. Organomegaly, may cause enlarged lymph |
| matous | nodes and may cause high white blood cell counts, high |
| | LDH and possible hypercalcaemia. |

Patients with chronic ATLL can be further divided into two sub-populations: favorable chronic and unfavorable chronic.

The methods for treating ATLL provided by the present disclosure can be used for treating any one of the subtypes of ATLL.

It is also hypothesized that an oligonucleotide inhibitor of miR-155 may interfere with the life cycle of a Human T-cell Lymphotropic Virus (HTLV). Accordingly, the present disclosure contemplates the use of an oligonucleotide inhibitor of miR-155 for treating HTLV infection itself or a disease associated with HTLV. There are four types of HTLVs—HTLV-1, HTLV-2, HTLV-3, and HTLV-4. Methods of the present disclosure can be used to treat a disease associated with any one of the sub-types of HTLVs. Exemplary diseases associated with a HTLV that may be treated with the methods of the present disclosure include, but are not limited to, ATLL, HTLV-1 associated myelopathy/tropical spastic paraparesis (HAM/TSP), and HTLV-associated dermatitis.

### Methods

In some embodiments, provided herein is a method for treating ATLL in a subject in need thereof, comprising: administering to the subject one or more therapeutic agents for treating ATLL; and administering an oligonucleotide inhibitor of miR-155 to the subject after the administration of the one or more therapeutic agents. In some of these embodiments, an oligonucleotide inhibitor of miR-155 is administered to the ATLL subject only after the administration of one or more therapeutic agents (i.e. the subject is not exposed to the oligonucleotide inhibitor of miR-155 prior to the treatment with one or more therapeutic agents) whereas in some other embodiments, an oligonucleotide inhibitor of miR-155 may be administered prior to or concurrently with the administration of one or more therapeutic agents.

In some embodiments, provided herein is a method for decreasing or maintaining the tumor cell count in a subject suffering from ATLL, comprising administering an oligonucleotide inhibitor of miR-155 to the subject, wherein the subject has received one or more therapeutic agents for treating ATLL prior to the treatment with the oligonucleotide inhibitor of miR-155.

In some embodiments, provided herein is a method for treating ATLL in a subject in need thereof, comprising administering to the subject an oligonucleotide inhibitor of miR-155, wherein the administration of the oligonucleotide inhibitor of miR-155 decreases the tumor cell count in the subject compared to the tumor cell count prior to the administration of the oligonucleotide inhibitor of miR-155 or maintains the tumor cell count at or below the level that was present at the beginning of the administration of the oligonucleotide inhibitor of miR-155.

In certain embodiments, the oligonucleotide inhibitor of miR-155 is administered to the subject in any one of the dosing amounts and/or according to any one of the dosing schedules disclosed herein.

In some embodiments, an oligonucleotide inhibitor of miR-155 administered to the subject to treat ATLL and/or to decrease or maintain the tumor cell count in a subject suffering from ATLL comprises the sequence of 5′-lCs.dAs.lCs.dGs.dAs.lTs.lTs.dAs.lGs.dCs.lAs.lTs.lTs.lA-3′ (SEQ ID NO: 25).

In some embodiments, an oligonucleotide inhibitor of miR-155 administered to the subject to treat ATLL and/or to decrease or maintain the tumor cell count in a subject suffering from ATLL comprises a sequence selected from Table 2.

As provided herein, in certain embodiments, the subject suffering from ATLL is treated with one or more therapeutic agents for ATLL prior to the treatment with the oligonucleotide inhibitor of miR-155. The treatment of the subject with one or more therapeutic agents for ATLL may decrease the number of tumor cells in the subject and the subsequent administration of an oligonucleotide inhibitor of miR-155 to the subject may further decrease the number of tumor cells in the subject. In some embodiments, the subsequent administration of an oligonucleotide inhibitor of miR-155 to the subject may decrease the number of tumor cells in the subject and then maintain the tumor cell count to the decreased level. Thus, administration of an oligonucleotide inhibitor of miR-155 to the subject treated with one or more therapeutic agents for ATLL prior to the treatment with the oligonucleotide inhibitor of miR-155 is expected to decrease the tumor cell count compared to the tumor cell count at the beginning of the treatment with antimiR-155 and maintain the tumor cell count at or below the decreased level.

In the embodiments where an oligonucleotide inhibitor of miR-155 is administered to the subject prior to the administration of one or more therapeutic agents, the oligonucleotide inhibitor of miR-155 may decrease the number of tumor cells in the subject and the subsequent administration of one or more therapeutic agents to the subject may further decrease the number of tumor cells in the subject. In these embodiments, the present disclosure contemplates continued administration of the oligonucleotide inhibitor of miR-155 to the subject even after the treatment with one or more therapeutic agents to further decrease the tumor cell count and/or to maintain the tumor cell count at or below the level provided by the treatment with one or more therapeutic agents.

The one or more therapeutic agents that may be administered to the subject for treating ATLL include, but are not limited to, zidovudine, interferon alpha, lenalidomide, etoposide, prednisone, vincristine, cyclophosphamide, doxorubicin hydrochloride, rituximab, romidepsin, mogamulizumab, pralatrexate, triamcinolone acetonide, ranimustine, vindesine, carboplatin, ALRN-6924 (a stapled alpha-helical peptide), methotrexate, gemcitabine, oxaliplatin, belinostat, acyclovir, Histone deacetylase (HDAC) inhibitors (HDIs) such as valproic acid, radiation therapy, and/or combinations thereof. In some embodiments, the one or more therapeutic agents described herein may be administered to the subject at least for one cycle prior to the administration of the oligonucleotide inhibitor of miR-155.

In some embodiments, one or more therapeutic agents administered to the subject for treating ATLL can be zidovudine, interferon alpha (e.g. interferon alpha-2b), lenalidomide, and the EPOCH chemotherapy. The EPOCH chemotherapy comprises administration of etoposide, prednisone, vincristine, cyclophosphamide, and doxorubicin hydrochloride. In some of these embodiments, the subject may be suffering from acute leukemic ATLL.

In some embodiments, one or more therapeutic agents administered to the subject for treating ATLL can be the CHOEP chemotherapy. In some embodiments, one or more therapeutic agents administered to the subject for treating ATLL can be the CHOEP chemotherapy and radiation therapy. The CHOEP chemotherapy comprises administration of cyclophosphamide, doxorubicin, vincristine, etoposide, and prednisone. In some of these embodiments, the subject may be suffering from acute ATLL or lymphomatous ATLL.

In some embodiments, one or more therapeutic agents administered to the subject for treating ATLL include methotrexate, the EPOCH chemotherapy, and lenalidomide. In some of these embodiments, the subject may be suffering from lymphomatous ATLL.

In some embodiments, one or more therapeutic agents administered to the subject for treating ATLL include lenalidomide and romidepsin. In some of these embodiments, the subject may be suffering from chronic unfavorable ATLL.

In some embodiments, one or more therapeutic agents administered to the subject for treating ATLL can be etoposide, zidovudine, interferon alpha, romidepsin, mogamulizumab, pralatrexate, lenalidomide, triamcinolone acetonide, and an inhibitor of primary p53 suppressor proteins MDMX and MDM2. An exemplary inhibitor of MDMX and MDM2 includes ALRN-6924, a lead product candidate being developed by Aileron Therapeutics. In some of these embodiments, the subject may be suffering from lymphomatous ATLL.

In some embodiments, one or more therapeutic agents that may be administered to the subject for treating ATLL can be VCAP-AMP-VECP chemotherapy (vincristine, cyclophosphamide, doxorubicin, and prednisone (VCAP), doxorubicin, ranimustine, and prednisone (AMP), and vindesine, etoposide, carboplatin, and prednisone (VECP)), zidovudine and interferon alpha (AZT/IFN), gemcitabine with oxaliplatin, belinostat, pralatrexate, intrathecal (IT) chemotherapy and zidovudine with valproic acid (AZT/VPA). In some of these embodiments, the subject may be suffering from lymphomatous ATLL.

In some embodiments, one or more therapeutic agents administered to the subject for treating ATLL can be phototherapy or photochemotherapy. The subject may have received a phototherapy or photochemotherapy prior to the administration of an oligonucleotide inhibitor of miR-155 or the subject may receive the phototherapy or photochemotherapy after and/or concurrently with the administration of an oligonucleotide inhibitor of miR-155 (also referred to herein as antimiR-155). The subject may also receive other therapeutic agents described herein in combination with the phototherapy or photochemotherapy. A phototherapy comprises exposing the skin of the subject to light waves, e.g. ultraviolet light or sunlight, according to a medical protocol. A photochemotherapy comprises a combination treatment using light waves and certain drugs. An exemplary photochemotherapy that the subject may receive is PUVA, which is a combination treatment comprising administration of Psoralens (P) and then exposing the skin to UVA (long wave ultraviolet radiation). A phototherapy or photochemotherapy is generally administered for treating skin lesions.

In some embodiments, administration of an oligonucleotide inhibitor of miR-155 to a subject decreases the tumor cell count compared to the tumor cell count at the beginning of the administration of the oligonucleotide inhibitor of miR-155. The term “tumor cell” refers to malignant T cells and includes CD4⁺ T cells, CD8⁺ T cells, αβ T cells, γδ T cells, memory T cells, and/or other subtypes of T cells.

In some embodiments, administration of an oligonucleotide inhibitor of miR-155 to a subject decreases the tumor cell count compared to the tumor cell count at the beginning of the administration of the oligonucleotide inhibitor of miR-155. In some embodiments, administration of an oligonucleotide inhibitor of miR-155 to a subject decreases the tumor cell count compared to the tumor cell count at the beginning of the administration of the oligonucleotide inhibitor of miR-155 and maintains the tumor cell count at or below the decreased level.

In some embodiments, administration of an oligonucleotide inhibitor of miR-155 to an ATLL subject maintains the tumor cell count at or below the level that was present at the beginning of the administration of the oligonucleotide inhibitor of miR-155.

In some embodiments, administration of an oligonucleotide inhibitor of miR-155 maintains the tumor cell count at or below the level that was present at the beginning of the administration of the oligonucleotide inhibitor of miR-155 for 3 months or more, 6 months or more, 9 months or more, 12 months or more, 15 months or more, 18 months or more, 24 months or more, 2.5 years or more, 3 years or more, 3.5 years or more, 4 years or more, 4.5 years or more, or 5 years or more.

In some embodiments, administration of an oligonucleotide inhibitor of miR-155 to an ATLL subject according to the present methods can decrease the tumor cell count by about 10%, 15%, 20%, 25%, 30%, 35%, 40%, 45%, 50%, 55%, 60%, 65%, 70%, 75%, 80%, 85%, or by about 90%, including values and ranges therebetween, compared to the tumor cell count at the beginning of the administration of the oligonucleotide inhibitor of miR-155.

In some embodiments, administration of an oligonucleotide inhibitor of miR-155 to an ATLL subject may reduce or inhibit proliferation of malignant T cells and/or induce apoptosis of malignant T cells. In some embodiments, proliferation of malignant T cells can be measured by measuring by the levels of cell cycle marker Ki67 using techniques such as flow cytometry. In some embodiments, administration of an oligonucleotide inhibitor of miR-155 to an ATLL subject may reduce or inhibit proliferation of malignant T cells by about 5%, 10%, 15%, 20%, 25%, 30%, 35%, 40%, 45%, 50%, 55%, 60%, 65%, 70%, 80%, 90% or 100%, including values therebetween, compared to the rate of proliferation at the beginning of the administration of the oligonucleotide inhibitor of miR-155.

In some embodiments, administration of an oligonucleotide inhibitor of miR-155 to an ATLL subject may increase apoptosis of malignant T cells by about 5%, 10%, 15%, 20%, 25%, 30%, 35%, 40%, 45%, 50%, 55%, 60%, 65%, 70%, 80%, 90% or 100%, including values therebetween, compared to the rate of apoptosis at the beginning of the administration of the oligonucleotide inhibitor of miR-155.

In some embodiments, administration of an oligonucleotide inhibitor of miR-155 to an ATLL subject may decrease the levels of cell cycle marker Ki67 on tumor cells by about 5%, 10%, 15%, 20%, 25%, 30%, 35%, 40%, 45%, 50%, 55%, 60%, 65%, 70%, 80%, 90% or 100%, including values therebetween, compared to the levels of Ki67 at the beginning of the administration of the oligonucleotide inhibitor of miR-155.

In some embodiments, administration of an oligonucleotide inhibitor of miR-155 to an ATLL subject may increase the levels of apoptosis marker cPARP on tumor cells by about 5%, 10%, 15%, 20%, 25%, 30%, 35%, 40%, 45%, 50%, 55%, 60%, 65%, 70%, 80%, 90% or 100%, including values therebetween, compared to the levels of cPARP at the beginning of the administration of the oligonucleotide inhibitor of miR-155.

In some embodiments, administration of an oligonucleotide inhibitor of miR-155 to an ATLL subject may reduce or inhibit proliferation of malignant T cells by about 5%, 10%, 15%, 20%, 25%, 30%, 35%, 40%, 45%, 50%, 55%, 60%, 65%, 70%, 80%, 90% or 100%, including values therebetween compared to the rate of proliferation of malignant T cells not treated with the oligonucleotide inhibitor of miR-155.

In some embodiments, administration of an oligonucleotide inhibitor of miR-155 to an ATLL subject may increase apoptosis of malignant T cells by about 5%, 10%, 15%, 20%, 25%, 30%, 35%, 40%, 45%, 50%, 55%, 60%, 65%, 70%, 80%, 90% or 100%, including values therebetween compared to the rate of apoptosis of malignant T cells not treated with the oligonucleotide inhibitor of miR-155.

In some embodiments, administration of an oligonucleotide inhibitor of miR-155 to an ATLL subject may decrease the activation level of malignant T cells. The level of activation of malignant T cells can be determined by measuring the levels of activation markers such as HLA-DR and/or CD69 using techniques such as flow cytometry. For example, a highly active malignant T cell is expected to express a greater number of HLA-DR and/or CD69 compared to a malignant T cell that is less active. In some embodiments, administration of an oligonucleotide inhibitor of miR-155 to an ATLL subject may decrease the activation of tumor cells by about 5%, 10%, 15%, 20%, 25%, 30%, 35%, 40%, 45%, 50%, 55%, 60%, 65%, 70%, 80%, 90% or 100%, including values therebetween, compared to the level of activation of tumor cells at the beginning of the administration of the oligonucleotide inhibitor of miR-155. In some embodiments, administration of an oligonucleotide inhibitor of miR-155 to an ATLL subject may decrease the activation of tumor cells by about 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 9 fold, or about 10 fold, including values therebetween, compared to the level of activation of tumor cells at the beginning of the administration of the oligonucleotide inhibitor of miR-155.

In some embodiments, administration of an oligonucleotide inhibitor of miR-155 to an ATLL subject may decrease the levels of activation markers such as HLA-DR and/or CD69 on tumor cells by about 5%, 10%, 15%, 20%, 25%, 30%, 35%, 40%, 45%, 50%, 55%, 60%, 65%, 70%, 75%, 80%, 85%, 90%, or 95%, including values therebetween, compared to the levels of HLA-DR and/or CD69 at the beginning of the administration of the oligonucleotide inhibitor of miR-155. In some embodiments, administration of an oligonucleotide inhibitor of miR-155 to an ATLL subject may decrease the levels of activation markers such as HLA-DR and/or CD69 on tumor cells by about 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 9 fold, or about 10 fold, including values therebetween, compared to the levels of activation markers at the beginning of the administration of the oligonucleotide inhibitor of miR-155.

In some embodiments, administration of an oligonucleotide inhibitor of miR-155 to an ATLL subject decreases the size of enlarged lymph nodes of the subject. In some embodiments, administration of the oligonucleotide inhibitor of miR-155 may decrease the size of enlarged lymph nodes of the subject to a normal size. In some embodiments, administration of an oligonucleotide inhibitor of miR-155 to an ATLL subject maintains the size of lymph nodes in the subject at about the current size, i.e. the size at the beginning of administration of an oligonucleotide inhibitor of miR-155.

In some embodiments, prior administration of one or more therapeutic agents to an ATLL subject may decrease the size of enlarged lymph nodes in the subject and the administration of the oligonucleotide inhibitor of miR-155 may further decrease the size of the enlarged lymph nodes and/or maintain the size of the lymph nodes to the decreased size.

In some embodiments, administration of an oligonucleotide inhibitor of miR-155 may decrease the size of an enlarged lymph node in the subject by about 5%, 10%, 15%, 20%, 25%, 30%, 35%, 40%, 45%, 50%, 55%, 60%, 65%, 70%, 80%, 90% or 100%, including values therebetween, compared to the size of the lymph node at the beginning of the administration of the oligonucleotide inhibitor of miR-155.

In some embodiments, administration of an oligonucleotide inhibitor of miR-155 may increase the progression-free survival of the subject by at least about 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 2.5 year, 3 years, 3.5 years, 4 years, 4.5 years, 5 years, 5.5 years, 6 years, 6.5 years, 7 years, 7.5 years, 8 years, 8.5 years, 9 years, 9.5 years, 10 years, or more. In some embodiments, administration of an oligonucleotide inhibitor of miR-155 to an ATLL subject may increase the progression-free survival of the subject by about 3 to 6 months or more, 4 to 8 months or more, 8 to 12 months or more, 6 to 12 months or more, 8 to 16 months or more, 10 to 15 months or more, 12 to 18 months or more, 12 to 24 months or more, 15 to 24 months or more, 18 to 24 months or more, 24 to 30 months or more, 1 to 2 years or more, 2 to 3 years or more, 2 to 4 years or more, 3 to 4 years or more, 3 to 5 years or more, 3 to 6 years or more, 2.5 years or more, 3 years or more, 3.5 years or more, 4 years or more, 4.5 years or more, 5 years or more, 6 years or more, 7 years or more, 8 years or more, 9 years or more, 10 years or more and the like.

In some embodiments, administration of an oligonucleotide inhibitor of miR-155 to a subject may reduce the HTLV load in the ATLL subject. For example, administration of an oligonucleotide inhibitor of miR-155 may reduce the HTLV load in the subject by about 5%, 10%, 15%, 20%, 25%, 30%, 35%, 40%, 45%, 50%, 55%, 60%, 65%, 70%, 75%, 80%, 85%, 90%, or 95%, including values therebetween, compared to the HTLV load at the beginning of the administration of the oligonucleotide inhibitor of miR-155.

In some embodiments, administration of an oligonucleotide inhibitor of miR-155 to a subject promotes restoration of normal homeostatic immune response in the subject. For example, in some embodiments, administration of an oligonucleotide inhibitor of miR-155 promotes re-population of healthy B and/or T cells in the subject. That is, in some embodiments, administration of an oligonucleotide inhibitor of miR-155 increases the number of healthy B and/or T cells in the subject. The increase in the number of healthy B and/or T cells can be by about 5%, 10%, 15%, 20%, 25%, 30%, 35%, 40%, 45%, 50%, 55%, 60%, 65%, 70%, 75%, 80%, 85%, 90%, or 95%, including values therebetween, compared to the number of healthy B and/or T cells at the beginning of the administration of the oligonucleotide inhibitor of miR-155.

In some embodiments, provided herein is a method for treating a disease associated with HTLV in a subject in need thereof, comprising administering to the subject an oligonucleotide inhibitor of miR-155. The disease associated with HTLV can be caused by HTLV-1, HTLV-2, HTLV-3, and/or HTLV-4. In exemplary embodiments, the disease associated with HTLV is selected from the group consisting of ATLL, HTLV-1 associated myelopathy/tropical spastic paraparesis (HAM/TSP), and HTLV-associated dermatitis.

Exemplary methods for treating ATLL comprising administering an oligonucleotide inhibitor of miR-155 are disclosed above. In another exemplary embodiment, provided herein is a method for treating HAM/TSP comprising administering an oligonucleotide inhibitor of miR-155. In another exemplary embodiment, provided herein is a method for treating HTLV-associated dermatitis comprising administering an oligonucleotide inhibitor of miR-155. In these embodiments, the oligonucleotide inhibitor of miR-155 can be administered in any one of the dosage amounts and according to any one of the schedules and routes of administration described throughout this disclosure.

In some embodiments, administration of an oligonucleotide inhibitor of miR-155 to a subject suffering from a disease associated with HTLV may reduce the HTLV load in the subject. For example, administration of an oligonucleotide inhibitor of miR-155 may reduce the HTLV load in the subject by about 5%, 10%, 15%, 20%, 25%, 30%, 35%, 40%, 45%, 50%, 55%, 60%, 65%, 70%, 75%, 80%, 85%, 90%, or 95%, including values therebetween, compared to the HTLV load at the beginning of the administration of the oligonucleotide inhibitor of miR-155.

In some embodiments, provided herein is a method for treating an HTLV infection in a subject in need thereof, comprising administering to the subject an oligonucleotide inhibitor of miR-155. The HTLV infection can be caused by HTLV-1, HTLV-2, HTLV-3, and/or HTLV-4. In some embodiments, administration of an oligonucleotide inhibitor of miR-155 to a subject suffering from an HTLV infection may reduce the HTLV load in the subject. For example, administration of an oligonucleotide inhibitor of miR-155 may reduce the HTLV load in the subject by about 5%, 10%, 15%, 20%, 25%, 30%, 35%, 40%, 45%, 50%, 55%, 60%, 65%, 70%, 75%, 80%, 85%, 90%, or 95%, including values therebetween, compared to the HTLV load at the beginning of the administration of the oligonucleotide inhibitor of miR-155.

Dosages, Schedules, and Routes of Administration of antimiR-155

In some embodiments, an antimiR-155 is administered according to a cycle or a schedule. In an exemplary embodiment, the cycle/schedule comprises (a) first administering at least one loading dose; and (b) second administering at least one maintenance dose.

In some embodiments, the cycle may be repeated 2, 3, 4, 5, 6, 7, 8, 9, 10, 15, 20, 25, 30, 35, 40, 45, or 50 times. In some embodiments, the loading dose may be repeated at least 2, 3, 4, 5, 6, 7, 8, 9, 10, 15, 20, 25, 30, 35, 40, 45, or 50 times. In some embodiments, the maintenance dose may be repeated at least 2, 3, 4, 5, 6, 7, 8, 9, 10, 15, 20, 25, 30, 35, 40, 45, or 50 times. The present disclosure also contemplates combinations of these embodiments. For example, in some embodiments, after administration of one loading dose and one maintenance dose, the maintenance dose may be repeated at least 2, 3, 4, 5, 6, 7, 8, 9, 10, 15, 20, 25, 30, 35, 40, 45, or 50 times. In some embodiments, the cycle may comprise administering one loading dose, followed by administering a maintenance dose at least 2 to 20 times and repeating the cycle for at least 2, 3, 4, 5, 6, 7, 8, 9, 10, 15, 20, 25, 30, 35, 40, 45, or 50 times.

In some embodiments, the loading dose may range from about 450 mg to about 4800 mg, about 450 mg to about 3600 mg, about 900 mg to about 4000 mg, about 900 mg to about 3600 mg, about 900 mg to about 3000 mg, about 900 mg to about 2500 mg, about 1250 mg to about 2750 mg, about 1500 mg to about 3000 mg, about 1500 mg to about 3600 mg, about 1500 mg to about 2400 mg, about 2000 mg to about 4000 mg, about 2000 mg to about 3600 mg, about 1800 mg to about 3600 mg, or about 1800 mg to about 2400 mg, including values and ranges therebetween.

In some embodiments, the loading dose can be about 450 mg, 750 mg, 900 mg, 1200 mg, 1400 mg, 1500 mg, 1600 mg, 1800 mg, 2000 mg, 2100 mg, 2200 mg, 2400 mg, 2600 mg, 2700 mg, 2800 mg, 3000 mg, 3200 mg, 3300 mg, 3400 mg, 3600 mg, 3800 mg, or about 4000 mg, including values and ranges therebetween. In some embodiments, the loading dose can be about 1500 mg, 1800 mg, 2100 mg, about 2400 mg, or about 3600 mg. In an exemplary embodiment, the loading dose is about 1800 mg. In another exemplary embodiment, the loading dose is about 3600 mg. In some embodiments, the loading dose is about 2 times, 3 times, 4 times, 5, 6 times, 8 times, or 10 times the maintenance dose.

In the present methods, the entire loading dose may be administered at once (a single bolus dose) or the loading dose can be split into 2, 3, 4, or 5 doses administered over a period of 7 to 10 days. In some embodiments, the loading dose can be split into equal doses, e.g., 3 equal doses and administered over a period of 7 days. In an exemplary embodiment, the loading dose can be split into equal doses, e.g., two or three doses administered every other day. In another exemplary embodiment, the loading dose can be split into equal doses administered on days 1, 3, and 5 of the cycle; or on days 1, 2, and 3 of the cycle; or on days, 1, 2, and 4 of the cycle, or on days 1, 3, and 6 of the cycle; or on days 1, 2, and 6 of the cycle; or on days 1, 2, and 5 of the cycle; or on days 1, 2, and 7 of the cycle; or on days 1, 3, and 7 of the cycle; or on days 1, 4, and 7 of the cycle and the like. In an exemplary embodiment, assuming the loading dose is about 1800 mg, it can be split into three doses with each split dose being about 600 mg administered every other day; or on three consecutive days; or on days, 1, 2, and 4 of the cycle, or on days 1, 3, and 6 of the cycle; or on days 1, 2, and 6 of the cycle; or on days 1, 2, and 5 of the cycle; or on days 1, 2, and 7 of the cycle; or on days 1, 3, and 7 of the cycle; or on days 1, 4, and 7 of the cycle and the like. In an exemplary embodiment, assuming the loading dose is about 3600 mg, it can be split into three doses with each split dose being about 1200 mg administered every other day; or on three consecutive days; or on days, 1, 2, and 4 of the cycle, or on days 1, 3, and 6 of the cycle; or on days 1, 2, and 6 of the cycle; or on days 1, 2, and 5 of the cycle; or on days 1, 2, and 7 of the cycle; or on days 1, 3, and 7 of the cycle; or on days 1, 4, and 7 of the cycle and the like. In another exemplary embodiment, assuming the loading dose is about 1800 mg, it can be split into two doses with each split dose being about 900 mg administered every other day. In another exemplary embodiment, assuming the loading dose is about 2400 mg, it can be split into three doses with each split dose being about 800 mg administered every other day. In yet another exemplary embodiment, assuming the loading dose is about 2400 mg, it can be split into two doses with each split dose being about 1200 mg administered every other day. In exemplary embodiments, administration every other day includes administering a dose on day 1, 3, 5, 7, 9, 11, 13, or 15 of the cycle and the like; or administering a dose on day 2, 4, 6, 8, 10, 12, 14, or 16 of the cycle and the like.

In some embodiments, the maintenance dose may range from about 150 mg to about 1800 mg, about 300 mg to about 2000 mg, about 300 mg to about 1800 mg, about 300 mg to about 1600 mg, about 300 mg to about 1200 mg, about 500 mg to about 1500 mg, about 500 mg to about 2000 mg, about 600 mg to about 2000 mg, about 600 mg to about 1800 mg, or about 600 mg to about 1500 mg, including values and ranges therebetween.

In some embodiments, the maintenance dose can be about 150 mg, 300 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or about 2000 mg, including values and ranges therebetween. In exemplary embodiments, the maintenance dose can be about 300 mg, 600 mg, 800 mg, 1000 mg, or about 1200 mg. In another exemplary embodiment, the maintenance dose is about 600 mg.

The maintenance dose can be administered once a week, twice a week, once in 10 days, once in two weeks, and the like. It is possible that the maintenance dose may be administered at short intervals at the beginning of the cycle and at larger intervals in the later part of the cycle. For example, at the beginning of the cycle, a maintenance dose may be administered at shorter intervals such as once every 3 or 4 days or twice a week and in the later part of the cycle, at larger intervals, such as once a week or once in 10 days. In an exemplary embodiment, the maintenance dose is administered once a week. In another exemplary embodiment, the maintenance dose is administered twice a week. In another exemplary embodiment, the maintenance dose is about 300 mg, 600 mg, 800 mg, 1000 mg, or about 1200 mg administered twice a week. In another exemplary embodiment, any of the maintenance dose amounts described herein can be administered twice a week for the first two months followed once a week administration. In another exemplary embodiment, the maintenance dose is about 600 mg administered once a week. In another exemplary embodiment, the maintenance dose is about 300 mg administered twice a week.

In some embodiments, an antimiR-155 is administered at a dose of about 0.5 mg/kg to 25 mg/kg, about 0.5 mg/kg to 20 mg/kg, about 1 mg/kg to 25 mg/kg, about 1 mg/kg to 20 mg/kg, about 0.5 mg/kg to 15 mg/kg, about 15 mg/kg to 15 mg/kg, including values and ranges therebetween. In some embodiments, an antimiR-155 is administered at a dose of about 0.5 mg/kg, 1 mg/kg, 2 mg/kg, 2.5 mg/kg, 3 mg/kg, 4 mg/kg, 5 mg/kg, 6 mg/kg, 7 mg/kg, 7.5 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, or 25 mg/kg, including values and ranges therebetween.

In some embodiments, an antimiR-155 is administered for at least 6 weeks, one month, 2 months, 3 months, 4 months, 5 months, 6 months, 8 months, 9 months, 10 months, 12 months, 15 months, 18 months, 2 years, 2.5 years, 3 years, 3.5 years, 4 years, 5 years, or 10 years or more. In these embodiments, an antimiR-155 can be administered in any one of the dosing amounts and/or according to any one of the dosing schedules disclosed herein. For example, in some embodiments, first a loading dose of about 1500 mg to about 2400 mg may be administered (bolus or split as described above) followed by administration of a maintenance dose of about 400 mg to about 1200 mg once a week for at least six months.

In the methods of the present disclosure, an oligonucleotide inhibitor of miR-155 can be administered orally, topically, parenterally, intradermally, subcutaneously, intravenously, intratumorally, intralesionally, or intramuscularly. In some embodiments, an oligonucleotide inhibitor of miR-155 is administered intravenously. In some embodiments, an oligonucleotide inhibitor of miR-155 is administered intratumorally or intralesionally. In some embodiments, an oligonucleotide inhibitor of miR-155 is administered subcutaneously.

### Pharmacokinetics

In some embodiments, the Cmax of an oligonucleotide inhibitor of miR-155 (“antimiR-155”) of the present disclosure, after a single dose 600 mg intravenous administration, is at least about 10 μg/mL, at least about 11 μg/mL, at least about 12 μg/mL, at least about 13 μg/mL, at least about 14 μg/mL, at least about 15 μg/mL, at least about 16 μg/mL, at least about 17 μg/mL, at least about 18 μg/mL, at least about 19 μg/mL, at least about 20 μg/mL, at least about 21 μg/mL, at least about 22 μg/mL, at least about 23 μg/mL, at least about 24 μg/mL, at least about 25 μg/mL, at least about 26 μg/mL, at least about 27 μg/mL, at least about 28 μg/mL, at least about 29 μg/mL, at least about 30 μg/mL, at least about 32 μg/mL, or at least about 35 μg/mL.

In some embodiments, the Cmax of an antimiR-155, after a 600 mg single dose intravenous administration, ranges from about 80% to about 125% of: about 10 μg/mL, about 12 μg/mL, about 14 μg/mL, about 16 μg/mL, about 18 μg/mL, about 19 μg/mL, about 20 μg/mL, about 21 μg/mL, about 22 μg/mL, about 23 μg/mL, about 24 μg/mL, about 25 μg/mL, about 26 μg/mL, about 27 μg/mL, about 28 μg/mL, about 29 μg/mL, about 30 μg/mL, about 32 μg/mL, or about 35 μg/mL.

In some embodiments, the Cmax of an antimiR-155, after a multiple dose intravenous administration of 600 mg, is at least about 10 μg/mL, at least about 11 μg/mL, at least about 12 μg/mL, at least about 13 μg/mL, at least about 14 μg/mL, at least about 15 μg/mL, at least about 16 μg/mL, at least about 17 μg/mL, at least about 18 μg/mL, at least about 19 μg/mL, at least about 20 μg/mL, at least about 21 μg/mL, at least about 22 μg/mL, at least about 23 μg/mL, at least about 24 μg/mL, at least about 25 μg/mL, at least about 26 μg/mL, at least about 27 μg/mL, at least about 28 μg/mL, at least about 29 μg/mL, at least about 30 μg/mL, at least about 32 μg/mL, or at least about 35 μg/mL.

In some embodiments, the Cmax of an antimiR-155, after a multiple dose intravenous administration of 600 mg, ranges from about 80% to about 125% of: about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, about 19 μg/mL, about 20 μg/mL, about 21 μg/mL, about 22 μg/mL, about 23 μg/mL, about 24 μg/mL, about 25 μg/mL, about 26 μg/mL, about 27 μg/mL, about 28 μg/mL, about 29 μg/mL, about 30 μg/mL, about 32 μg/mL, or about 35 μg/mL.

In some embodiments, the tₘₐₓ of an antimiR-155 after 600 mg single dose intravenous administration is about 1.8 h or greater, 1.9 h or greater, 2 h or greater, 2.1 h or greater, 2.2 h or greater, 2.3 h or greater, 2.4 h or greater or about 2.5 h or greater. In some embodiments, the tₘₐₓ of an antimiR-155 after 600 mg single dose intravenous administration of the antimiR-155 is about 1.9 h or greater.

In some embodiments, the tₘₐₓ of an antimiR-155 after 600 mg multiple dose intravenous administration of the antimiR-155 is about 1.8 h or greater, 1.9 h or greater, 2 h or greater, 2.1 h or greater, 2.2 h or greater, 2.3 h or greater, 2.4 h or greater or about 2.5 h or greater. In some embodiments, the tₘₐₓ of an antimiR-155 after 600 mg multiple dose intravenous administration is about 1.9 h or greater.

In some embodiments, an antimiR-155 provides an AUC₀₋₂₄, after 600 mg single dose intravenous administration, of at least about 50 μg*hr/mL, at least about 55 μg*hr/mL, at least about 60 μg*hr/mL, at least about 65 μg*hr/mL, at least about 70 μg*hr/mL, at least about 75 μg*hr/mL, at least about 80 μg*hr/mL, at least about 85 μg*hr/mL, at least about 90 μg*hr/mL, at least about 95 μg*hr/mL, at least about 100 μg*hr/mL, at least about 105 μg*hr/mL, at least about 110 μg*hr/mL, at least about 115 μg*hr/mL, or at least about 120 μg*hr/mL.

In some embodiments, the AUC₀₋₂₄ of an antimiR-155, after 600 mg single dose intravenous administration, ranges from about 80% to about 125% of: about 50 μg*hr/mL, about 55 μg*hr/mL, about 60 μg*hr/mL, about 65 μg*hr/mL, about 70 μg*hr/mL, about 75 μg*hr/mL, about 80 μg*hr/mL, about 85 μg*hr/mL, about 90 μg*hr/mL, about 95 μg*hr/mL, about 100 μg*hr/mL, about 105 μg*hr/mL, about 110 μg*hr/mL, about 115 μg*hr/mL, or about 120 μg*hr/mL.

In some embodiments, an antimiR-155 provides an AUC₀₋₂₄, after 600 mg multiple dose intravenous administration, of at least about 40 μg*hr/mL, at least about 45 μg*hr/mL, at least about 50 μg*hr/mL, at least about 55 μg*hr/mL, at least about 60 μg*hr/mL, at least about 65 μg*hr/mL, at least about 70 μg*hr/mL, at least about 75 μg*hr/mL, at least about 80 μg*hr/mL, at least about 85 μg*hr/mL, at least about 90 μg*hr/mL, at least about 95 μg*hr/mL, at least about 100 μg*hr/mL, at least about 105 μg*hr/mL, at least about 110 μg*hr/mL, at least about 115 μg*hr/mL, or at least about 120 μg*hr/mL.

In some embodiments, the AUC₀₋₂₄ of an antimiR-155 after 600 mg multiple dose intravenous administration ranges from about 80% to about 125% of: about 40 μg*hr/mL, about 45 μg*hr/mL, about 50 μg*hr/mL, about 55 μg*hr/mL, about 60 μg*hr/mL, about 65 μg*hr/mL, about 70 μg*hr/mL, about 75 μg*hr/mL, about 80 μg*hr/mL, about 85 μg*hr/mL, about 90 μg*hr/mL, about 95 μg*hr/mL, about 100 μg*hr/mL, about 105 μg*hr/mL, about 110 μg*hr/mL, about 115 μg*hr/mL, or about 120 μg*hr/mL.

In some embodiments, an AUC_{inf} of an antimiR-155 of the present disclosure after a 600 mg single dose intravenous administration is at least about 40 μg*hr/mL, at least about 45 μg*hr/mL, at least about 50 μg*hr/mL, at least about 55 μg*hr/mL, at least about 60 μg*hr/mL, at least about 65 μg*hr/mL, at least about 70 μg*hr/mL, at least about 75 μg*hr/mL, at least about 80 μg*hr/mL, at least about 85 μg*hr/mL, at least about 90 μg*hr/mL, at least about 95 μg*hr/mL, at least about 100 μg*hr/mL, at least about 105 μg*hr/mL, at least about 110 μg*hr/mL, at least about 115 μg*hr/mL, or at least about 120 μg*hr/mL.

In some embodiments, an AUC_{inf} of an antimiR-155 after a 600 mg single dose intravenous administration ranges from about 80% to about 125% of: about 40 μg*hr/mL, about 45 μg*hr/mL, about 50 μg*hr/mL, about 55 μg*hr/mL, about 60 μg*hr/mL, about 65 μg*hr/mL, about 70 μg*hr/mL, about 75 μg*hr/mL, about 80 μg*hr/mL, about 85 μg*hr/mL, about 90 μg*hr/mL, about 95 μg*hr/mL, about 100 μg*hr/mL, about 105 μg*hr/mL, about 110 μg*hr/mL, about 115 μg*hr/mL, or about 120 μg*hr/mL.

In some embodiments, an AUC_{inf} of an antimiR-155 after 600 mg multiple dose intravenous administration is at least about 40 μg*hr/mL, at least about 45 μg*hr/mL, at least about 50 μg*hr/mL, at least about 55 μg*hr/mL, at least about 60 μg*hr/mL, at least about 65 μg*hr/mL, at least about 70 μg*hr/mL, at least about 75 μg*hr/mL, at least about 80 μg*hr/mL, at least about 85 μg*hr/mL, at least about 90 μg*hr/mL, at least about 95 μg*hr/mL, at least about 100 μg*hr/mL, at least about 105 μg*hr/mL, at least about 110 μg*hr/mL, at least about 115 μg*hr/mL, or at least about 120 μg*hr/mL.

In some embodiments, an AUC_{inf} of an antimiR-155 after a 600 mg single dose intravenous administration ranges from about 80% to about 125% of: about 40 μg*hr/mL, about 45 μg*hr/mL, about 50 μg*hr/mL, about 55 μg*hr/mL, about 60 μg*hr/mL, about 65 μg*hr/mL, about 70 μg*hr/mL, about 75 μg*hr/mL, about 80 μg*hr/mL, about 85 μg*hr/mL, about 90 μg*hr/mL, about 95 μg*hr/mL, about 100 μg*hr/mL, about 105 μg*hr/mL, about 110 μg*hr/mL, about 115 μg*hr/mL, or about 120 μg*hr/mL.

In some embodiments, an antimiR-155 comprising the sequence of SEQ ID NO: 25 provides any of the Cmax, tₘₐₓ, AUC₀₋₂₄, and AUC_{inf} values disclosed herein.

Oligonucleotide Inhibitors of miR-155

In some embodiments, an oligonucleotide inhibitor of miR-155 used in the present methods has a length of 11 to 16, 11 to 15, or 11 to 14 nucleotides. For example, the oligonucleotide inhibitor of miR-155 used in the present methods can be 11, 12, 13, 14, 15, or 16 nucleotides in length. In some embodiments, the oligonucleotide inhibitor of miR-155 has a length of 12 or 14 nucleotides.

In some embodiments, the sequence of an oligonucleotide inhibitor of miR-155 is sufficiently complementary to a mature sequence of miR-155-5p to hybridize to miR-155-5p under physiological conditions and inhibit the activity or function of miR-155-5p in the cells of a subject. For instance, in some embodiments, an oligonucleotide inhibitor comprises a sequence that is at least partially complementary to a mature sequence of miR-155-5p, e.g. at least about 75%, 80%, 85%, 90%, 95%, 96%, 97%, 98%, or 99% complementary to a mature sequence of miR-155-5p. In some embodiments, the oligonucleotide inhibitor can be substantially complementary to a mature sequence of miR-155-5p, that is at least about 90%, 95%, 96%, 97%, 98%, or 99% complementary to a mature sequence of miR-155-5p. In one embodiment, the oligonucleotide inhibitor comprises a sequence that is 100% or fully complementary to a mature sequence of miR-155-5p. It is understood that the sequence of the oligonucleotide inhibitor is considered complementary to miR-155 even if the oligonucleotide sequence includes a modified nucleotide instead of a naturally occurring nucleotide. For example, if a mature sequence of miR-155 comprises a guanosine nucleotide at a specific position, the oligonucleotide inhibitor may comprise a modified cytidine nucleotide, such as a locked cytidine nucleotide or 2′-fluoro-cytidine, at the corresponding position.

In some embodiments, the entire sequence of the oligonucleotide inhibitor of miR-155 is fully complementary to a mature sequence of human miR-155-5p. In various embodiments, the mature sequence of human miR-155-5p to which the sequence of the oligonucleotide inhibitor of the present invention is partially, substantially, or fully complementary to includes nucleotides 1-17, or nucleotides 2-17, or nucleotides 2-16, or nucleotides 2-15, or nucleotides 2-14, or nucleotides 2-13, or nucleotides 2-12 from the 5′ end of SEQ ID NO: 1. In one embodiment, the mature sequence of human miR-155-5p to which the sequence of the oligonucleotide inhibitor of the present invention is partially, substantially, or fully complementary to includes nucleotides 2-15 from the 5′ end of SEQ ID NO: 1. In another embodiment, the mature sequence of human miR-155-5p to which the sequence of the oligonucleotide inhibitor of the present invention is partially, substantially, or fully complementary to includes nucleotides 2-13 from the 5′ end of SEQ ID NO: 1.

In some embodiments, the oligonucleotide inhibitor of miR-155 contains at least one backbone modification, such as at least one phosphorothioate, morpholino, or phosphonocarboxylate internucleotide linkage (see, for example, U.S. Pat. Nos. 6,693,187 and 7,067,641, which are herein incorporated by reference in their entireties). In certain embodiments, the oligonucleotide inhibitor of miR-155 is fully phosphorothioate-linked.

In some embodiments, the oligonucleotide inhibitor of miR-155 contains at least one modified nucleotide. In some embodiments, the oligonucleotide inhibitor contains at least 5, 6, 7, 8, 9, 10, or more modified nucleotides. The term “modified nucleotide” as used herein encompasses nucleotides with sugar, base, and/or backbone modifications. Examples of modified nucleotides include, but are not limited to, locked nucleotides (LNA), ethylene-bridged nucleotides (ENA), 2′-C-bridged bicyclic nucleotide (CBBN), 2′,4′-constrained ethyl nucleic acid called S-cEt or cEt, 2′-4′-carbocyclic LNA, and 2′ substituted nucleotides.

The terms “locked nucleotide,” “locked nucleic acid unit,” “locked nucleic acid residue,” or “LNA unit” may be used interchangeably throughout the disclosure and refer to a bicyclic nucleoside analogue. For instance, suitable oligonucleotide inhibitors can be comprised of one or more “conformationally constrained” or bicyclic sugar nucleoside modifications (BSN) that confer enhanced thermal stability to complexes formed between the oligonucleotide containing BSN and their complementary target strand. In one embodiment, the oligonucleotide inhibitors contain locked nucleotides or LNAs containing the 2′-O, 4′-C-methylene ribonucleoside (structure A) wherein the ribose sugar moiety is in a “locked” conformation. In another embodiment, the oligonucleotide inhibitors contain at least one 2′-C, 4′-C-bridged 2′ deoxyribonucleoside (structure B). See, e.g., U.S. Pat. No. 6,403,566 and Wang et al. (1999) Bioorganic and Medicinal Chemistry Letters, Vol. 9: 1147-1150, both of which are herein incorporated by reference in their entireties. In yet another embodiment, the oligonucleotide inhibitors contain at least one modified nucleoside having the structure shown in structure C. The oligonucleotide inhibitors targeting miR-155 can contain combinations of BSN (LNA, 2′-C, 4′-C-bridged 2′ deoxyribonucleoside, and the like) or other modified nucleotides, and ribonucleotides or deoxyribonucleotides.

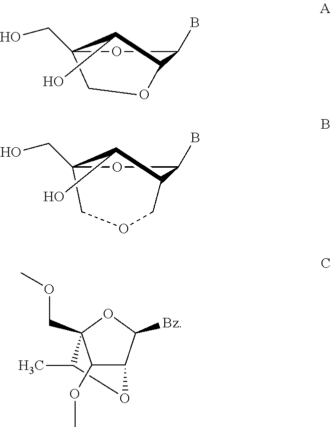

The terms “non-LNA nucleotide”, and “non-LNA modification” as used herein refer to a nucleotide different from a LNA nucleotide, i.e. the terms include a DNA nucleotide, an RNA nucleotide as well as a modified nucleotide where a base and/or sugar is modified except that the modification is not a LNA modification.

In some embodiments, the oligonucleotide inhibitor of miR-155 contains at least one nucleotide containing a non-LNA modification. For example, in one embodiment, the oligonucleotide inhibitor of miR-155 contains at least one 2′-C-bridged bicyclic nucleotide (CBBN) as described in U.S. Pre-Grant Publication No. 2016/0010090A1 (“the '090 publication”), which is hereby incorporated by reference herein in its entirety. The '090 publication describes a variety of CBBN modifications such as 2′-CBBN, oxoCBBN, amino CBBN, thioCBBN, etc. All CBBN modifications described in the '090 publications could be used in the oligonucleotide inhibitors of the present invention. In another embodiment, the non-LNA modification present in the oligonucleotide inhibitor of miR-155 could be an ethylene-bridged nucleic acid (ENA) modification. For example, in one embodiment, the oligonucleotide inhibitor of miR-155 contains at least one ethylene-bridged nucleic acid (ENA), also referred to herein as ethylene-bridged nucleotide. Other bridged modifications include 2′,4′-constrained ethyl nucleic acid called S-cEt or cEt and 2′-4′-carbocyclic LNA (carba-LNA).

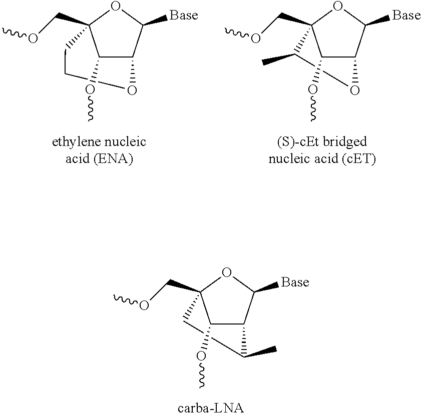

When referring to substituting a DNA or RNA nucleotide by its corresponding locked nucleotide in the context of the present invention, the term “corresponding locked nucleotide” is intended to mean that the DNA/RNA nucleotide has been replaced by a locked nucleotide containing the same naturally-occurring nitrogenous base as the DNA/RNA nucleotide that it has replaced or the same nitrogenous base that is chemically modified. For example, the corresponding locked nucleotide of a DNA nucleotide containing the nitrogenous base C may contain the same nitrogenous base C or the same nitrogenous base C that is chemically modified, such as 5-methylcytosine.

In certain embodiments, the oligonucleotide inhibitor of miR-155 contains at least 5, 6, 7, 8, 9, 10, or 11 locked nucleotides. In one embodiment, the oligonucleotide inhibitor of miR-155 contains at least 7, 8, 9, or 10 locked nucleotides. In one embodiment, at least the first three nucleotides from the 3′ end of the oligonucleotide inhibitor are locked nucleotides. In another embodiment, at least the first four nucleotides from the 3′ end of the oligonucleotide inhibitor are locked nucleotides. In yet another embodiment, the first nucleotide from the 5′ end of the oligonucleotide inhibitor is a locked nucleotide.

In certain embodiments, the oligonucleotide inhibitor contains at least 1, at least 2, at least 3, at least 4, or at least 5 DNA nucleotides. In one embodiment, at least the second nucleotide from the 5′ end of the oligonucleotide inhibitor is a DNA nucleotide. In another embodiment, at least the second and fourth nucleotides from the 5′ end of the oligonucleotide inhibitor are DNA nucleotides.

Oligonucleotide inhibitors of the present invention may include modified nucleotides that have a base modification or substitution. The natural or unmodified bases in RNA are the purine bases adenine (A) and guanine (G), and the pyrimidine bases cytosine (C) and uracil (U) (DNA has thymine (T)). Modified bases, also referred to as heterocyclic base moieties, include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (including 5-bromo, 5-trifluoromethyl and other 5-substituted uracils and cytosines), 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. In certain embodiments, oligonucleotide inhibitors targeting miR-155 comprise one or more BSN modifications in combination with a base modification (e.g. 5-methylcytosine).

Oligonucleotide inhibitors of the present invention may include nucleotides with modified sugar moieties. Representative modified sugars include carbocyclic or acyclic sugars, sugars having substituent groups at one or more of their 2′,3′ or 4′ positions and sugars having substituents in place of one or more hydrogen atoms of the sugar. In certain embodiments, the sugar is modified by having a substituent group at the 2′ position. In additional embodiments, the sugar is modified by having a substituent group at the 3′ position. In other embodiments, the sugar is modified by having a substituent group at the 4′ position. It is also contemplated that a sugar may have a modification at more than one of those positions, or that an oligonucleotide inhibitor may have one or more nucleotides with a sugar modification at one position and also one or more nucleotides with a sugar modification at a different position.

Sugar modifications contemplated in the oligonucleotide inhibitors of the present invention include, but are not limited to, a substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted with C₁ to C₁₀ alkyl or C₂ to C₁₀ alkenyl and alkynyl. In one embodiment, the modification includes 2′-methoxyethoxy (2′-O—CH₂CH₂OCH₃, which is also known as 2′-O-(2-methoxyethyl) or 2′-MOE), that is, an alkoxyalkoxy group. Another modification includes 2′-dimethylaminooxyethoxy, that is, a O(CH₂)₂ON(CH₃)₂ group, also known as 2′-DMAOE and 2′-dimethylaminoethoxyethoxy (also known in the art as 2′-O-dimethyl-amino-ethoxy-ethyl or 2′-DMAEOE), that is, 2′-O—CH₂—O—CH₂—N(CH₃)₂.

Additional sugar substituent groups include allyl (—CH₂—CH═CH₂), —O-allyl, methoxy (—O—CH₃), aminopropoxy (—OCH₂CH₂CH₂NH₂), and fluoro (F). Sugar substituent groups on the 2′ position (2′-) may be in the arabino (up) position or ribo (down) position. One 2′-arabino modification is 2′-F. Other similar modifications may also be made at other positions on the sugar moiety, particularly the 3′ position of the sugar on the 3′ terminal nucleoside or in 2′-5′ linked oligonucleotides and the 5′ position of 5′ terminal nucleotide. In certain embodiments, the sugar modification is a 2′-O-alkyl (e.g. 2′-O-methyl, 2′-O-methoxyethyl), 2′-halo (e.g., 2′-fluoro, 2′-chloro, 2′-bromo), and 4′ thio modifications.

Other modifications of oligonucleotide inhibitors to enhance stability and improve efficacy, such as those described in U.S. Pat. No. 6,838,283, which is herein incorporated by reference in its entirety, are known in the art and are suitable for use in the methods of the invention. For instance, to facilitate in vivo delivery and stability, the oligonucleotide inhibitor can be linked to a steroid, such as cholesterol moiety, a vitamin, a fatty acid, a carbohydrate or glycoside, a peptide, or other small molecule ligand at its 3′ end.

In some embodiments, the oligonucleotide inhibitors of the present invention may be conjugated to a carrier molecule such as a steroid (cholesterol). The carrier molecule is attached to the 3′ or 5′ end of the oligonucleotide inhibitor either directly or through a linker or a spacer group. In various embodiments, the carrier molecule is cholesterol, a cholesterol derivative, cholic acid or a cholic acid derivative. The use of carrier molecules disclosed in U.S. Pat. No. 7,202,227, which is incorporated by reference herein in its entirety, is also envisioned. In certain embodiments, the carrier molecule is cholesterol and it is attached to the 3′ or 5′ end of the oligonucleotide inhibitor through at least a six carbon linker. In some embodiments, the carrier molecule is attached to the 3′ or 5′ end of the oligonucleotide inhibitor through a six or nine carbon linker. In some embodiments, the linker is a cleavable linker. In various embodiments, the linker comprises a substantially linear hydrocarbon moiety. The hydrocarbon moiety may comprise from about 3 to about 15 carbon atoms and may be conjugated to cholesterol through a relatively non-polar group such as an ether or a thioether linkage. In certain embodiments, the hydrocarbon linker/spacer comprises an optionally substituted C2 to C15 saturated or unsaturated hydrocarbon chain (e.g. alkylene or alkenylene). A variety of linker/spacer groups described in U.S. Pre-grant Publication No. 2012/0128761, which is incorporated by reference herein in its entirety, can be used in the present invention.

In one embodiment, the oligonucleotide inhibitor of miR-155 comprises a sequence of 11 to 16 nucleotides, wherein the oligonucleotide inhibitor is fully complementary to a mature sequence of miR-155 and has a full phosphorothioate backbone; and wherein at least the first three nucleotides from the 3′ end of the oligonucleotide inhibitor are locked nucleotides and at least the second nucleotide from the 5′ end of the oligonucleotide inhibitor is a deoxyribonucleic acid (DNA) nucleotide. In some of these embodiments, the fourth nucleotide from the 3′ end of the oligonucleotide inhibitor is also a locked nucleotide. In some of these embodiments, at least the second and fourth nucleotides from the 5′ end of the oligonucleotide inhibitor are DNA nucleotides. In certain embodiments, the oligonucleotide inhibitor of miR-155 has a length of 12 or 14 nucleotides. In some embodiments, the oligonucleotide inhibitor contains at least 5, 6, 7, 8, 9, or 10 locked nucleotides. In further embodiments, at least the sixth and/or the eighth nucleotide from the 5′ end of the oligonucleotide inhibitor is a DNA nucleotide. In yet further embodiments, the oligonucleotide inhibitor comprises DNA nucleotides at the second, sixth, and the eighth position from the 5′ end.

In another embodiment, the oligonucleotide inhibitor of miR-155 comprises a sequence of 11 to 14 nucleotides, wherein the oligonucleotide inhibitor is fully complementary to a mature sequence of miR-155 and has a full phosphorothioate backbone; and wherein at least the first three nucleotides from the 3′ end of said oligonucleotide inhibitor are modified nucleotides and at least the second nucleotide from the 5′ end of the oligonucleotide inhibitor is a modified or an unmodified deoxyribonucleic acid (DNA) nucleotide.

In yet another embodiment, the oligonucleotide inhibitor of miR-155 comprises a sequence of 11 to 14 nucleotides, wherein the oligonucleotide inhibitor is fully complementary to a mature sequence of miR-155 and has a full phosphorothioate backbone; wherein at least 7 nucleotides of said oligonucleotide inhibitor are modified nucleotides and at least the second nucleotide from the 5′ end of the oligonucleotide inhibitor is a modified or an unmodified deoxyribonucleic acid (DNA) nucleotide.

In yet another embodiment, the oligonucleotide inhibitor of miR-155 comprises a sequence of 11 to 14 nucleotides, wherein the oligonucleotide inhibitor is fully complementary to a mature sequence of miR-155 and has a full phosphorothioate backbone; and wherein at least the first three nucleotides from 3′ end of said oligonucleotide inhibitor are modified nucleotides and at least the fourth and fifth nucleotides from the 5′ end of the oligonucleotide inhibitor are modified or unmodified deoxyribonucleic acid (DNA) nucleotides. In some of these embodiments, the fourth and/or the fifth DNA nucleotide from the 5′ end of the oligonucleotide inhibitor are unmodified DNA nucleotides.

In some embodiments where the oligonucleotide inhibitor is 11 to 14 nucleotides long, said inhibitor contains at least 5, 6, 7, 8, 9, or 10 modified nucleotides. In some of these embodiments, the oligonucleotide inhibitor contains 7, 8, 9, or 10 modified nucleotides. In some embodiments where the oligonucleotide inhibitor is 11 to 14 nucleotides long, at least the first three nucleotides from the 3′ end of said oligonucleotide inhibitor are modified nucleotides. In some embodiments, all modified nucleotides are locked nucleotides. In some embodiments, the 5, 6, 7, 8, 9, or 10 modified nucleotides present in the oligonucleotide inhibitors are a combination of locked nucleotides and nucleotides containing non-LNA modifications such as ethylene-bridged nucleotides, 2′-C-bridged bicyclic nucleotides, 2′-substituted nucleotides, and other sugar and/or base modifications described herein.

In some embodiments, the second nucleotide from the 5′ end of the oligonucleotide inhibitor is an unmodified deoxyribonucleic acid (DNA) nucleotide.

In an exemplary embodiment, the oligonucleotide inhibitor of miR-155 administered to a subject according to the present methods comprises a sequence of SEQ ID NO: 25.

In some embodiments, the oligonucleotide inhibitor of miR-155 administered to a subject according to the present methods comprises a sequence selected from Table 2.

TABLE 2

| SEQ ID | Sequence (5′-3′) |
|---|---|
| NO. | with modifications¹ |
| SEQ ID | 5′-lAs.dTs.dCs.dAs.lCs.lGs.dAs.lTs. |
| NO: 3 | dTs.lAs.lGs.dCs.lAs.dTs.lTs.lA-3′ |
| SEQ ID | 5′-lAs.dTs.dCs.dAs.lCs.lGs.dAs.dTs. |
| NO: 4 | lTs.lAs.lGs.dCs.lAs.dTs.lTs.lA-3′ |
| SEQ ID | 5′-lAs.lTs.dCs.dAs.dCs.lGs.dAs.lTs. |
| NO: 5 | dTs.lAs.lGs.dCs.lAs.dTs.lTs.lA-3′ |
| SEQ ID | 5′-lAs.lTs.dCs.dAs.dCs.lGs.lAs.dTs. |
| NO: 6 | dTs.lAs.lGs.lCs.dAs.lTs.dTs.lA-3′ |
| SEQ ID | 5′-lAs.dTs.dCs.dAs.lCs.lGs.dAs.lTs. |
| NO: 7 | dTs.lAs.lGs.dCs.lAs.lTs.dTs.lA-3′ |
| SEQ ID | 5′-lAs.lTs.dCs.dAs.lCs.dGs.dAs.dTs. |
| NO: 8 | lTs.lAs.dGs.lCs.lAs.dTs.lTs.lA-3′ |
| SEQ ID | 5-lAs.dTs.dCs.dAs.lCs.dGs.lAs.dTs.l |
| NO: 9 | Ts.lAs.dGs.lCs.lAs.dTs.lTs.lA-3 |
| SEQ ID | 5′-lAs.dTs.dCs.lAs.dCs.dGs.lAs.lTs. |
| NO: 10 | dTs.lAs.lGs.dCs.lAs.dTs.lTs.lA-3′ |
| SEQ ID | 5′-lAs.dTs.lCs.dAs.dCs.lGs.dAs.lTs. |
| NO: 11 | lTs.dAs.dGs.lCs.lAs.dTs.lTs.lA-3′ |
| SEQ ID | 5′-lAs.lTs.dCs.lAs.lCs.dGs.dAs.dTs. |
| NO: 12 | lTs.lAs.dGs.lCs.lAs.dTs.dTs.lA-3′ |
| SEQ ID | 5′-lAs.dTs.lCs.dAs.dCs.dGs.lAs.dTs. |
| NO: 13 | lTs.lAs.dGs.lCs.lAs.dTs.lTs.lA-3′ |
| SEQ ID | 5′-lAs.dTs.lCs.dAs.lCs.dGs.lAs.dTs. |
| NO: 14 | lTs.dAs.lGs.dCs.lAs.dTs.lTs.lA-3′ |
| SEQ ID | 5′-lTs.dCs.dAs.lCs.dGs.dAs.lTs.dTs. |
| NO: 15 | dAs.lGs.dCs.lAs.lTs.dTs.lA-3′ |
| SEQ ID | 5′-lTs.dCs.lAs.dCs.dGs.lAs.lTs.dTs. |
| NO: 16 | dAs.lGs.dCs.lAs.dTs.lTs.lA-3′ |
| SEQ ID | 5′-lTs.dCs.dAs.dCs.lGs.lAs.lTs.dTs. |
| NO: 17 | dAs.lGs.dCs.lAs.dTs.lTs.lA-3′ |
| SEQ ID | 5′-lTs.lCs.lAs.dCs.lGs.dAs.dTs.lTs. |
| NO: 18 | lAs.dGs.lCs.dAs.dTs.lTs.lA-3′ |
| SEQ ID | 5′-lTs.dCs.dAs.lCs.dGs.dAs.dTs.lTs. |
| NO: 19 | lAs.lGs.lCs.lAs.lTs.lTs.lA-3′ |
| SEQ ID | 5′-lTs.dCs.lAs.dCs.lGs.lAs.lTs.dTs. |
| NO: 20 | dAs.lGs.lCs.lAs.dTs.lTs.lA-3′ |
| SEQ ID | 5′-lGs.lAs.lTs.lTs.lAs.lGs.dCs.lAs. |
| NO: 21 | lTs.dTs.lA-3′ |
| SEQ ID | 5′-lCs.dGs.lAs.lTs.lTs.lAs.lGs.dCs. |
| NO: 22 | lAs.lTs.lTs.lA-3′ |
| SEQ ID | 5′-lCs.dGs.lAs.lTs.lTs.dAs.lGs.dCs. |
| NO: 23 | lAs.lTs.lTs.lA-3′ |
| SEQ ID | 5′-lCs.lAs.dCs.lGs.dAs.lTs.lTs.dAs. |
| NO: 24 | lGs.dCs.lAs.lTs.lTs.lA-3′ |
| SEQ ID | 5′-lCs.dAs.lCs.dGs.dAs.lTs.lTs.dAs. |
| NO: 25 | lGs.dCs.lAs.lTs.lTs.lA-3′ |
| SEQ ID | 5′-lTs.dCs.lAs.mdCs.lGs.lAs.lTs.dTs. |
| NO: 26 | dAs.lGs.lCs.lAs.dTs.lTs.lA-3′ |
| SEQ ID | 5′-lTs.lAs.lGs.lCs.lAs.lTs.lTs.lA-3′ |
| NO: 27 | |
| SEQ ID | 5′-lCs.dAs.lCs.dGs.lAs.lTs.lTs.dAs. |
| NO: 29 | lGs.dCs.lAs.lTs.lTs.lA-3′ |
| SEQ ID | 5′-lCs.dAs.lCs.dGs.lAs.dTs.lTs.dAs. |
| NO: 30 | lGs.dCs.lAs.lTs.lTs.lA-3′ |
| SEQ ID | 5′-lCs.dAs.lCs.dGs.dAs.lTs.lTs.lAs. |
| NO: 31 | lGs.dCs.lAs.dTs.lTs.lA-3′ |
| SEQ ID | 5′-dCs.dAs.lCs.dGs.dAs.lTs.lTs.dAs. |
| NO: 32 | lGs.dCs.lAs.lTs.lTs.lA-3′ |
| SEQ ID | 5′-lCs.lAs.lCs.dGs.dAs.lTs.lTs.dAs. |
| NO: 33 | lGs.dCs.lAs.lTs.lTs.lA-3′ |
| SEQ ID | 5′-lCs.dAs.dCs.dGs.dAs.lTs.lTs.dAs. |
| NO: 34 | lGs.dCs.lAs.lTs.lTs.lA-3′ |
| SEQ ID | 5′-lCs.dAs.lCs.lGs.dAs.lTs.lTs.dAs. |
| NO: 35 | lGs.dCs.lAs.lTs.lTs.lA-3′ |
| SEQ ID | 5′-lCs.dAs.lCs.dGs.lAs.lTs.lTs.dAs. |
| NO: 36 | lGs.dCs.lAs.lTs.lTs.lA-3′ |
| SEQ ID | 5′-lCs.dAs.lCs.dGs.dAs.dTs.lTs.dAs. |
| NO: 37 | lGs.dCs.lAs.lTs.lTs.lA-3′ |
| SEQ ID | 5′-lCs.dAs.lCs.dGs.dAs.lTs.dTs.dAs. |
| NO: 38 | lGs.dCs.lAs.lTs.lTs.lA-3′ |
| SEQ ID | 5′-lCs.dAs.lCs.dGs.dAs.lTs.lTs.lAs. |
| NO: 39 | lGs.dCs.lAs.lTs.lTs.lA-3′ |
| SEQ ID | 5′-lCs.dAs.lCs.dGs.dAs.lTs.lTs.dAs. |
| NO: 40 | dGs.dCs.lAs.lTs.lTs.lA-3′ |
| SEQ ID | 5′-lCs.dAs.lCs.dGs.dAs.lTs.lTs.dAs. |
| NO: 41 | lGs.lCs.lAs.lTs.lTs.lA-3′ |
| SEQ ID | 5′-lCs.dAs.lCs.dGs.dAs.lTs.lTs.dAs. |
| NO: 42 | lGs.dCs.dAs.lTs.lTs.lA-3′ |
| SEQ ID | 5′-lCs.dAs.lCs.dGs.dAs.lTs.lTs.dAs. |
| NO: 43 | lGs.dCs.lAs.dTs.lTs.lA-3′ |
| SEQ ID | 5′-lCs.dAs.lCs.dGs.dAs.lTs.lTs.dAs. |
| NO: 44 | lGs.dCs.lAs.lTs.dTs.lA-3′ |
| SEQ ID | 5′-lCs.dAs.lCs.dGs.dAs.lTs.lTs.dAs. |
| NO: 45 | lGs.dCs.lAs.lTs.lTs.dA-3′ |
| SEQ ID | 5′-dCs.lAs.lCs.dGs.dAs.lTs.lTs.dAs. |
| NO: 46 | lGs.dCs.lAs.lTs.lTs.lA-3′ |
| SEQ ID | 5′-lCs.lAs.dCs.dGs.dAs.lTs.lTs.dAs. |
| NO: 47 | lGs.dCs.lAs.lTs.lTs.lA-3′ |
| SEQ ID | 5′-lCs.dAs.dCs.lGs.dAs.lTs.lTs.dAs. |
| NO: 48 | lGs.dCs.lAs.lTs.lTs.lA-3′ |
| SEQ ID | 5′-lCs.dAs.lCs.dGs.lAs.dTs.lTs.dAs. |
| NO: 49 | lGs.dCs.lAs.lTs.lTs.lA-3′ |
| SEQ ID | 5′-lCs.dAs.lCs.dGs.dAs.lTs.dTs.lAs. |
| NO: 50 | lGs.dCs.lAs.lTs.lTs.lA-3′ |
| SEQ ID | 5′-lCs.dAs.lCs.dGs.dAs.lTs.lTs.lAs. |
| NO: 51 | dGs.dCs.lAs.lTs.lTs.lA-3′ |
| SEQ ID | 5′-lCs.dAs.lCs.dGs.dAs.lTs.lTs.dAs. |
| NO: 52 | dGs.lCs.lAs.lTs.lTs.lA-3′ |
| SEQ ID | 5′-lCs.dAs.lCs.dGs.dAs.lTs.lTs.dAs. |
| NO: 53 | lGs.lCs.dAs.lTs.lTs.lA-3′ |
| SEQ ID | 5′-lAs.lCs.dGs.dAs.lTs.lTs.dAs.lGs. |
| NO: 54 | dCs.lAs.lTs.lTs.lA-3′ |
| SEQ ID | 5′-lCs.dGs.dAs.lTs.lTs.dAs.lGs.dCs. |
| NO: 55 | lAs.lTs.lTs.lA-3′ |
| SEQ ID | 5′-lGs.dAs.lTs.lTs.dAs.lGs.dCs.lAs. |
| NO: 56 | lTs.lTs.lA-3′ |
| SEQ ID | 5′-dAs.lCs.dGs.dAs.lTs.lTs.dAs.lGs. |
| NO: 57 | dCs.lAs.lTs.lTs.lA-3′ |
| SEQ ID | 5′-lAs.dCs.dGs.dAs.lTs.lTs.dAs.lGs. |
| NO: 58 | dCs.lAs.lTs.lTs.lA-3′ |
| SEQ ID | 5′-lAs.lCs.lGs.dAs.lTs.lTs.dAs.lGs. |
| NO: 59 | dCs.lAs.lTs.lTs.lA-3′ |
| SEQ ID | 5′-lAs.lCs.dGs.lAs.lTs.lTs.dAs.lGs. |
| NO: 60 | dCs.lAs.lTs.lTs.lA-3′ |
| SEQ ID | 5′-lAs.lCs.dGs.dAs.dTs.lTs.dAs.lGs. |
| NO: 61 | dCs.lAs.lTs.lTs.lA-3′ |
| SEQ ID | 5′-lAs.lCs.dGs.dAs.lTs.dTs.dAs.lGs. |
| NO: 62 | dCs.lAs.lTs.lTs.lA-3′ |
| SEQ ID | 5′-lAs.lCs.dGs.dAs.lTs.lTs.lAs.lGs. |
| NO: 63 | dCs.lAs.lTs.lTs.lA-3′ |
| SEQ ID | 5′-lAs.lCs.dGs.dAs.lTs.lTs.dAs.dGs. |
| NO: 64 | dCs.lAs.lTs.lTs.lA-3′ |
| SEQ ID | 5′-lAs.lCs.dGs.dAs.lTs.lTs.dAs.lGs. |
| NO: 65 | lCs.lAs.lTs.lTs.lA-3′ |
| SEQ ID | 5′-lAs.lCs.dGs.dAs.lTs.lTs.dAs.lGs. |
| NO: 66 | dCs.dAs.lTs.lTs.lA-3′ |
| SEQ ID | 5′-lAs.lCs.dGs.dAs.lTs.lTs.dAs.lGs. |
| NO: 67 | dCs.lAs.dTs.lTs.lA-3′ |
| SEQ ID | 5′-lAs.lCs.dGs.dAs.lTs.lTs.dAs.lGs. |
| NO: 68 | dCs.lAs.lTs.dTs.lA-3′ |
| SEQ ID | 5′-lAs.lCs.dGs.dAs.lTs.lTs.dAs.lGs. |
| NO: 69 | dCs.lAs.lTs.lTs.dA-3′ |
| SEQ ID | 5′-lAs.dCs.lGs.dAs.lTs.lTs.dAs.lGs. |
| NO: 70 | dCs.lAs.lTs.lTs.lA-3′ |
| SEQ ID | 5′-lAs.lCs.dGs.lAs.dTs.lTs.dAs.lGs. |
| NO: 71 | dCs.lAs.lTs.lTs.lA-3′ |
| SEQ ID | 5′-lAs.lCs.dGs.dAs.lTs.lTs.dAs.lGs. |
| NO: 72 | dCs.lAs.lTs.lTs.lA-3′ |
| SEQ ID | 5′-lAs.lCs.dGs.dAs.lTs.dTs.lAs.lGs. |
| NO: 73 | dCs.lAs.lTs.lTs.lA-3′ |
| SEQ ID | 5′-lAs.lCs.dGs.dAs.lTs.lTs.lAs.dGs. |
| NO: 74 | dCs.lAs.lTs.lTs.lA-3′ |
| SEQ ID | 5′-lAs.lCs.dGs.dAs.lTs.lTs.dAs.dGs. |
| NO: 75 | lCs.lAs.lTs.lTs.lA-3′ |
| SEQ ID | 5′-lAs.lCs.dGs.dAs.lTs.lTs.dAs.lGs. |
| NO: 76 | lCs.dAs.lTs.lTs.lA-3′ |
| SEQ ID | 5′-dCs.dGs.dAs.lTs.lTs.dAs.lGs.dCs. |
| NO: 77 | lAs.lTs.lTs.lA-3′ |
| SEQ ID | 5′-lCs.lGs.dAs.lTs.lTs.dAs.lGs.dCs. |
| NO: 78 | lAs.lTs.lTs.lA-3′ |
| SEQ ID | 5′-lCs.dGs.lAs.lTs.lTs.dAs.lGs.dCs. |
| NO: 79 | lAs.lTs.lTs.lA-3′ |
| SEQ ID | 5′-lCs.dGs.dAs.dTs.lTs.dAs.lGs.dCs. |
| NO: 80 | lAs.lTs.lTs.lA-3′ |
| SEQ ID | 5′-lCs.dGs.dAs.lTs.dTs.dAs.lGs.dCs. |
| NO: 81 | lAs.lTs.lTs.lA-3′ |
| SEQ ID | 5′-lCs.dGs.dAs.lTs.lTs.lAs.lGs.dCs. |
| NO: 82 | lAs.lTs.lTs.lA-3′ |
| SEQ ID | 5′-lCs.dGs.dAs.lTs.lTs.dAs.dGs.dCs. |
| NO: 83 | lAs.lTs.lTs.lA-3′ |
| SEQ ID | 5′-lCs.dGs.dAs.lTs.lTs.dAs.lGs.lCs. |
| NO: 84 | lAs.lTs.lTs.lA-3′ |
| SEQ ID | 5′-lCs.dGs.dAs.lTs.lTs.dAs.lGs.dCs. |
| NO: 85 | dAs.lTs.lTs.lA-3′ |
| SEQ ID | 5′-lCs.dGs.dAs.lTs.lTs.dAs.lGs.dCs. |
| NO: 86 | lAs.dTs.lTs.lA-3′ |
| SEQ ID | 5′-lCs.dGs.dAs.lTs.lTs.dAs.lGs.dCs. |
| NO: 87 | lAs.lTs.dTs.lA-3′ |
| SEQ ID | 5′-lCs.dGs.dAs.lTs.lTs.dAs.lGs.dCs. |
| NO: 88 | lAs.lTs.lTs.dA-3′ |
| SEQ ID | 5′-dCs.lGs.dAs.lTs.lTs.dAs.lGs.dCs. |
| NO: 89 | lAs.lTs.lTs.lA-3′ |
| SEQ ID | 5′-lCs.dGs.lAs.dTs.lTs.dAs.lGs.dCs. |
| NO: 90 | lAs.lTs.lTs.lA-3′ |
| SEQ ID | 5′-lCs.dGs.dAs.lTs.dTs.lAs.lGs.dCs. |
| NO: 91 | lAs.lTs.lTs.lA-3′ |
| SEQ ID | 5′-lCs.dGs.dAs.lTs.lTs.lAs.dGs.dCs. |
| NO: 92 | lAs.lTs.lTs.lA-3′ |
| SEQ ID | 5′-lCs.dGs.dAs.lTs.lTs.dAs.dGs.lCs. |
| NO: 93 | lAs.lTs.lTs.lA-3′ |
| SEQ ID | 5′-lCs.dGs.dAs.lTs.lTs.dAs.lGs.lCs. |
| NO: 94 | dAs.lTs.lTs.lA-3′ |
| SEQ ID | 5′-dGs.dAs.lTs.lTs.dAs.lGs.dCs.lAs. |
| NO: 95 | lTs.lTs.lA-3′ |
| SEQ ID | 5′-lGs.lAs.lTs.lTs.dAs.lGs.dCs.lAs. |
| NO: 96 | lTs.lTs.lA-3′ |
| SEQ ID | 5′-lGs.dAs.dTs.lTs.dAs.lGs.dCs.lAs. |
| NO: 97 | lTs.lTs.lA-3′ |
| SEQ ID | 5′-lGs.dAs.lTs.dTs.dAs.lGs.dCs.lAs. |
| NO: 98 | lTs.lTs.lA-3′ |
| SEQ ID | 5′-lGs.dAs.lTs.lTs.lAs.lGs.dCs.lAs. |
| NO: 99 | lTs.lTs.lA-3′ |
| SEQ ID | 5′-lGs.dAs.lTs.lTs.dAs.dGs.dCs.lAs. |
| NO: | lTs.lTs.lA-3′ |
| 100 | |
| SEQ ID | 5′-lGs.dAs.lTs.lTs.dAs.lGs.lCs.lAs. |
| NO: | lTs.lTs.lA-3′ |
| 101 | |
| SEQ ID | 5′-lGs.dAs.lTs.lTs.dAs.lGs.dCs.dAs. |
| NO: | lTs.lTs.lA-3′ |
| 102 | |
| SEQ ID | 5′-lGs.dAs.lTs.lTs.dAs.lGs.dCs.lAs. |
| NO: | dTs.lTs.lA-3′ |
| 103 | |
| SEQ ID | 5′-lGs.dAs.lTs.lTs.dAs.lGs.dCs.lAs. |
| NO: | lTs.dTs.lA-3′ |
| 104 | |
| SEQ ID | 5′-lGs.dAs.lTs.lTs.dAs.lGs.dCs.lAs. |
| NO: | lTs.lTs.dA-3′ |
| 105 | |
| SEQ ID | 5′-dGs.lAs.lTs.lTs.dAs.lGs.dCs.lAs. |
| NO: | lTs.lTs.lA-3′ |
| 106 | |
| SEQ ID | 5′-lGs.lAs.dTs.lTs.dAs.lGs.dCs.lAs. |
| NO: | lTs.lTs.lA-3′ |
| 107 | |
| SEQ ID | 5′-lGs.dAs.lTs.dTs.lAs.lGs.dCs.lAs. |
| NO: | lTs.lTs.lA-3′ |
| 108 | |
| SEQ ID | 5′-lGs.dAs.lTs.lTs.lAs.dGs.dCs.lAs. |
| NO: | lTs.lTs.lA-3′ |
| 109 | |
| SEQ ID | 5′-lGs.dAs.lTs.lTs.dAs.dGs.lCs.lAs. |
| NO: | lTs.lTs.lA-3′ |
| 110 | |
| SEQ ID | 5′-lGs.dAs.lTs.lTs.dAs.lGs.lCs.dAs. |
| NO: | lTs.lTs.lA-3′ |
| 111 | |
| SEQ ID | 5′-eCs.dAs.eCs.dGs.dAs.eTs.eTs.dAs. |
| NO: | eGs.dCs.eAs.eTs.eTs.eA-3′ |
| 112 | |
| SEQ ID | 5′-lCs.dAs.lCs.dGs.dAs.lTs.lTs.dAs. |
| NO: | lGs.dCs.eAs.lTs.lTs.eA-3′ |
| 113 | |
| SEQ ID | 5′-eCs.dAs.eCs.dGs.dAs.lTs.lTs.dAs. |
| NO: | lGs.dCs.lAs.lTs.lTs.lA-3′ |
| 114 | |
| SEQ ID | 5′-lCs.dAs.lCs.dGs.dAs.lTs.lTs.dAs. |
| NO: | eGs.dCs.lAs.lTs.lTs.lA-3′ |
| 115 | |
| SEQ ID | 5′-lCs.dAs.lCs.dGs.dAs.eTs.eTs.dAs. |
| NO: | lGs.dCs.lAs.eTs.eTs.lA-3′ |
| 116 | |
| SEQ ID | 5′-lCs.dAs.lCs.dGs.dAs.lTs.eTs.dAs. |
| NO: | lGs.dCs.lAs.lTs.lTs.lA-3′ |
| 117 | |
| SEQ ID | 5′-lCs.dAs.lCs.dGs.dAs.lTs.lTs.dAs. |
| NO: | lGs.dCs.lAs.lTs.eTs.lA-3′ |
| 118 | |
| SEQ ID | 5′-lCs.dAs.lCs.dGs.dAs.lTs.lTs.dAs. |
| NO: | lGs.dCs.abAs.lTs.lTs.abA-3′ |
| 119 | |
| SEQ ID | 5′-abCs.dAs.abCs.dGs.dAs.abTs.abTs. |
| NO: | dAs.abGs.dCs.abAs.abTs.abTs.abA- |
| 120 | 3′ |

¹l = locked nucleic acid modification; d = deoxyribonucleotide; s = phosphorothioate linkage; md = 5-Methylcytosine; e = ethylene-bridged nucleotide (ENA); ab = amino-2′-C-Bridged Bicyclic Nucleotide (CBBN).

In some embodiments, an oligonucleotide inhibitors of miR-155 can be administered in a naked form, i.e. the oligonucleotide itself or a composition comprising the oligonucleotide is administered. In some other embodiments, an oligonucleotide inhibitor of miR-155 can be administered to the subject by delivering to the target cell (malignant T cells) an expression vector encoding the miR-155 oligonucleotide inhibitor. A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, and the like. In one particular embodiment, the viral vector is a lentiviral vector or an adenoviral vector. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In one embodiment, an expression vector for expressing an oligonucleotide inhibitor of miR-155 comprises a promoter operably linked to a polynucleotide sequence encoding the oligonucleotide inhibitor. The phrase “operably linked” or “under transcriptional control” as used herein means that the promoter is in the correct location and orientation in relation to a polynucleotide to control the initiation of transcription by RNA polymerase and expression of the polynucleotide.

As used herein, a “promoter” refers to a DNA sequence recognized by the synthetic machinery of the cell, or introduced synthetic machinery, required to initiate the specific transcription of a gene. Suitable promoters include, but are not limited to RNA pol I, pol II, pol III, and viral promoters (e.g. human cytomegalovirus (CMV) immediate early gene promoter, the SV40 early promoter, and the Rous sarcoma virus long terminal repeat). In one embodiment, the promoter is a T-cell specific promoter such as the proximal and distal promoters of the lck gene or promoter and enhancer sequences of the CD4 gene, etc.

In certain embodiments, the promoter operably linked to a polynucleotide encoding a miR-155 oligonucleotide inhibitor can be an inducible promoter. Inducible promoters are known in the art and include, but are not limited to, tetracycline promoter, metallothionein IIA promoter, heat shock promoter, steroid/thyroid hormone/retinoic acid response elements, the adenovirus late promoter, and the inducible mouse mammary tumor virus LTR.

Methods of delivering expression constructs and nucleic acids to cells are known in the art and can include, for example, calcium phosphate co-precipitation, electroporation, microinjection, DEAE-dextran, lipofection, transfection employing polyamine transfection reagents, cell sonication, gene bombardment using high velocity microprojectiles, and receptor-mediated transfection.

### Kits

The present disclosure also provides kits for treating ATLL or HTLV-associated diseases. In an exemplary embodiment, the kit for treating ATLL may comprise any of the oligonucleotide inhibitors of miR-155 described herein and one or more therapeutic agents described herein. In another exemplary embodiment, the kit for treating ATLL may comprise an expression vector comprising a nucleotide sequence for any of the oligonucleotide inhibitors of miR-155 described herein and one or more therapeutic agents described herein.

This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

All patent and non-patent documents referenced throughout this disclosure are incorporated by reference herein in their entirety.

### EXAMPLES

### Example 1: Phase I Clinical Trial Study of ATLL Patients Treated with antimiR-155

Eight patients with ATLL have been enrolled in an ongoing clinical study to analyze the effects of treatment with an antimiR-155 inhibitor. Prior to the treatment with antimiR-155, each of the eight patients received one or more therapeutic agents for ATLL as described in detail below.

Table 3 shows the demographic and baseline characteristics of the patients enrolled in the clinical study.

TABLE 3

| Demographic | n = 8* | |
|---|---|---|
| Sex | | |
| Male (n, %) | 5 | (63%) |
| Female (n, %) | 3 | (37%) |
| Age | | |
| Median years (range) | 50 | (40-68) |
| Race | | |
| Black | 7 | (88%) |
| Not reported | 1 | (17%) |
| Ethnicity | | |
| Non-Hispanic | 8 | (100%) |
| ATLL Type | | |
| Acute ATLL | 3 | (38%) |
| Lymphomatous ATLL | 4 | (50%) |
| Chronic Unfavorable | 1 | (12%) |
| Prior Systemic Therapies | | |
| # Patients Reporting | 8 | |
| Median # (range) | 3 | (1-10) |

*Subject 102-012 re-enrolled as 102-015

For all patients, a loading dose of an antimiR-155 comprising the sequence of SEQ ID NO: 25 (referred to as “cobomarsen” in the figures) was administered followed by once a week maintenance dose. The loading dose was 1800 mg split into three equal doses of about 600 mg, each administered on days 1, 3, and 5 of the treatment cycle followed by a maintenance dose of 600 mg once a week. The first maintenance dose was administered 7 days after the last loading dose. The antimiR-155 was administered as an intravenous infusion over the period of 2 hours.

Patient No. 101-008 with Acute Leukemic ATLL: A 49 year old male patient was diagnosed with acute ATLL in December 2016 and was treated with zidovudine (AZT), interferon alfa-2b (IFN), lenalidomide, and EPOCH chemotherapy for about 9 months. The disease was not well controlled with these therapeutic agents. After completing chemotherapy on Oct. 16, 2017, the patient had a low tumor burden but had not achieved a complete response. Approximately 3 weeks after the last dose of chemotherapy, the first dose of the antimiR-155 was administered to the patient according to the schedule and the dose noted above.

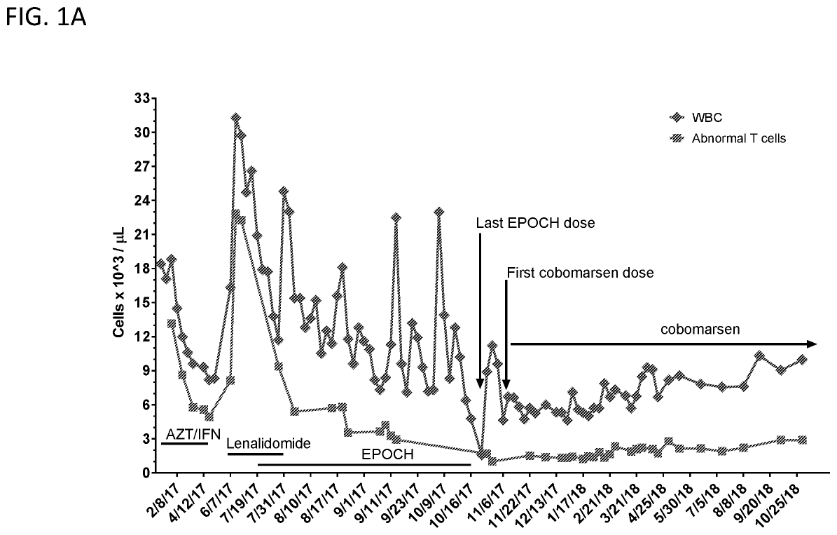

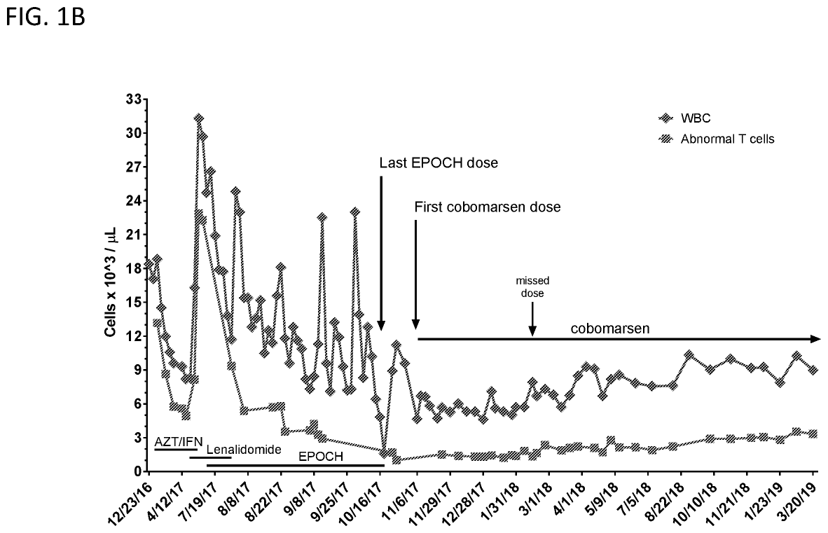

During the time the patient is receiving treatment with the antimiR-155, no other therapeutic agents for treating ATLL are being administered. The antimiR-155 treatment has stabilized the tumor cell count in peripheral blood for over 12 months. FIG. 1A shows the tumor cell count and the WBC count from the beginning of the treatment until filing of the provisional application. FIG. 1B shows the tumor cell count and the WBC count from the beginning of the treatment until filing of the present application. Although the data points until filing of the provisional application are the same for FIGS. 1A and 1B, the dates on the X-axis of FIGS. 1A and 1B do not match as these dates are auto-generated by the analysis software and change as the number of data entries change.

The patient had enlarged lymph nodes even after treatment with zidovudine, interferon alfa-2b, lenalidomide, and EPOCH chemotherapy. The antimiR-155 treatment has resulted in normalization of still enlarged lymph node after chemotherapy (from 1.0 cm to 0.8 cm) as measured by CT Scan done on Oct. 31, 2017 compared to the CT scan performed on Jan. 2, 2018. The size of the lymph node has remained normal as of last CT scan on Aug. 22, 2018. At the time the provisional application was filed, adverse events reported as possibly or definitely related to study drug included ALT/AST (Grade 1) and intermittent diarrhea (Grade 1). The updated records for this patient at the time of filing the present application do not list ALT/AST (Grade 1) as the adverse events.

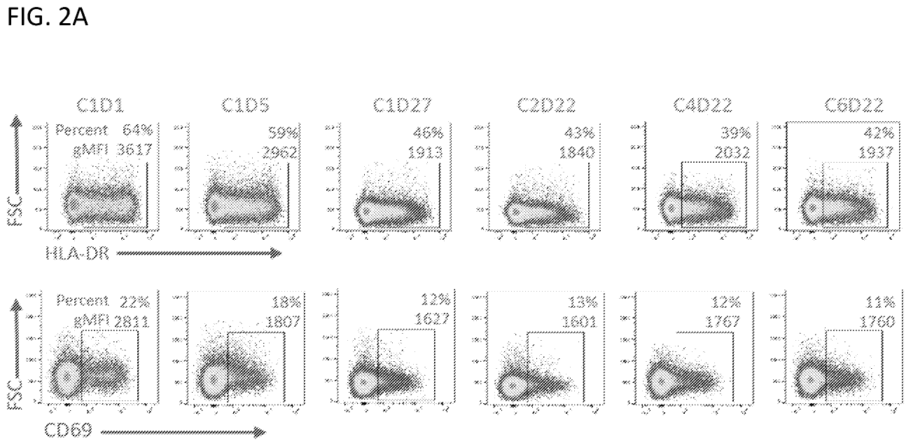

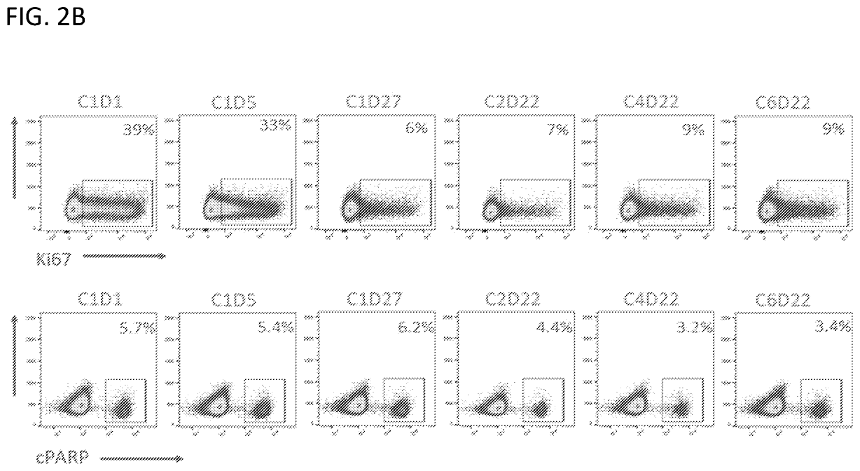

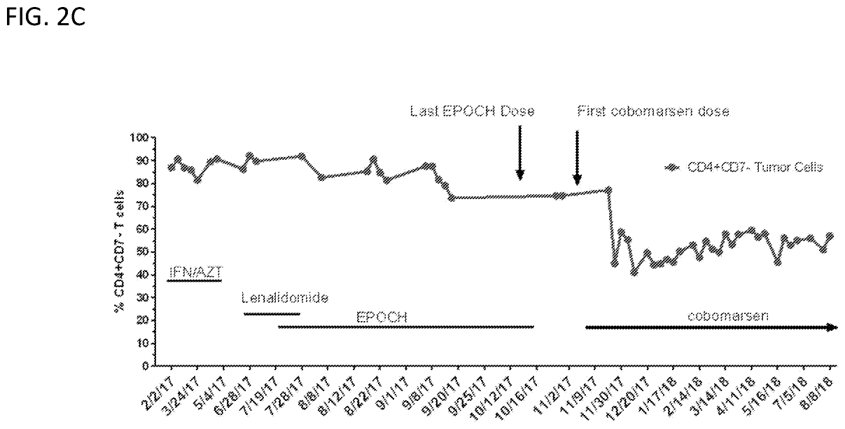

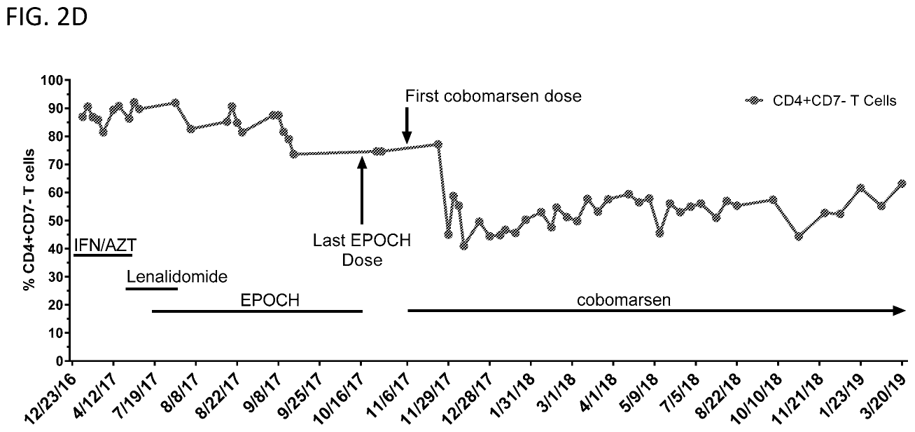

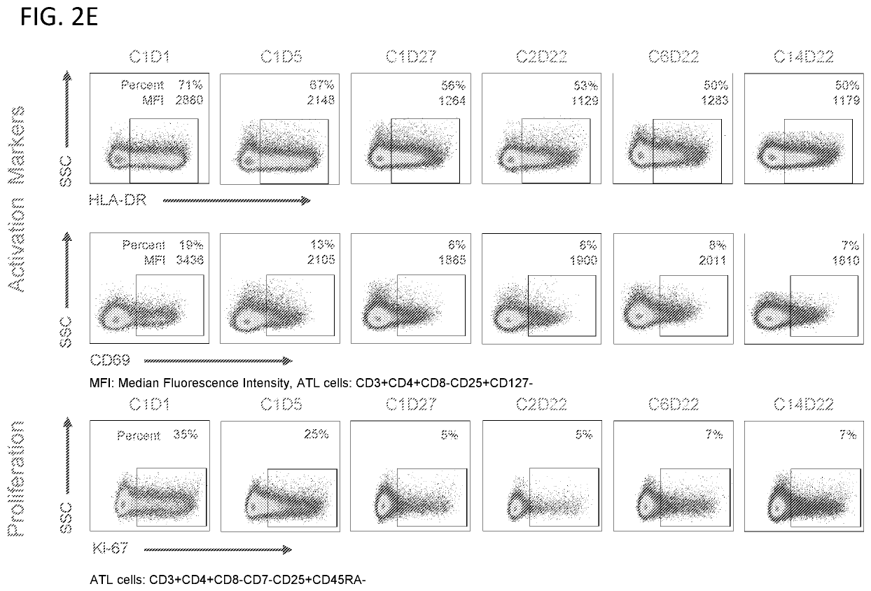

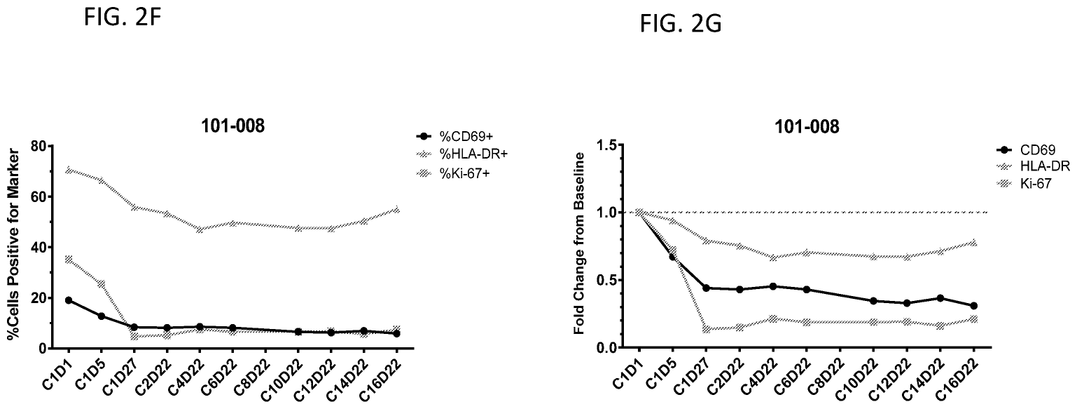

Additionally, the antimiR-155 treatment has decreased the activation and proliferation status of circulating tumor cells in the patient as measured by the mean fluorescent intensity (MFI) and percent tumor cell count for activation markers HLA-DR and CD69 and proliferation marker Ki67 using flow cytometry. FIGS. 2A and 2B are the as-filed figures from the provisional application showing the flow cytometric assessment of the markers from the start of the antimiR-155 treatment until filing of the provisional application. FIG. 2A shows the decreased level (MFI) and decreased percent positive tumor cells as measured by activation markers HLA-DR and CD69 after the anitimiR-155 treatment. In FIG. 2, the notation C1D1 stands for Cycle 1 Day 1, the notation C1D5 stands for Cycle 1 Day 5, and so forth. FIG. 2B, upper panel, shows the cell cycle marker Ki67 in tumor cells decreased by the end of the first cycle of antimiR-155 and has remained low for at least 12 months of treatment. FIG. 2B, lower panel, shows the apoptosis marker cPARP in peripheral tumor cells did not change throughout the antimiR-155 treatment.

FIG. 2C shows the percentage of tumor cells from the beginning of the ATLL treatment until the filing of the provisional application. FIG. 2D shows the percentage of tumor cells from the beginning of the ATLL treatment until the filing of the present application. FIGS. 2C-2D show that the anitimiR-155 treatment decreased the percent tumor cells count circulating in the peripheral blood compared to the tumor cell count percentage at the beginning of administration of antimiR-155 and continued administration of the antimiR-155 has maintained the tumor cell count percentage to the decreased level.

FIG. 2E shows the flow cytometric assessment of the markers from the start of the antimiR-155 treatment until filing of the present application. Although the same raw data for the data points C1D1, C1D5, C1D27, C2D22, and C6D22 was used to generate FIGS. 2A and 2B and part of FIG. 2E, the percentages (% positive cells for the marker) and the MFI for these data points in FIGS. 2A and 2B differ slightly from the percentages and the MFI shown for these data points in FIG. 2E. It is known that the percentages and the MFI for the flow cytometry data points depend on how the gates are set at the time of analysis. One of ordinary skill in the art understands that it is difficult to set the same, exact gates every time the raw data is analyzed. In other words, although the raw data remains the same, the percentages and the MFI can vary depending on how the gates are set at the time of analysis. Although the percentages and the MFI differ slightly between FIGS. 2A and 2B versus FIG. 2E, it is clear from the figures that the percent positive cells and the MFI for the HLA-DR, CD69, and Ki67 markers decrease over time upon treatment with the antimiR-155.

FIGS. 2F and 2G show the percentage of ATL cells positive for each biomarker (FIG. 2F) and the fold change from baseline (C1D1) (FIG. 2G) over the course of the antimiR-155 treatment.

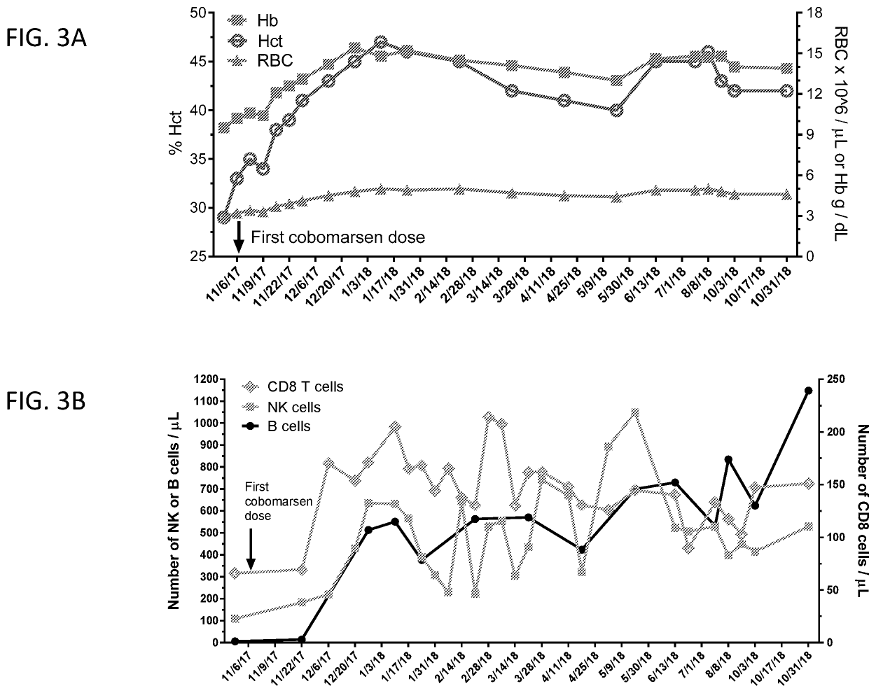

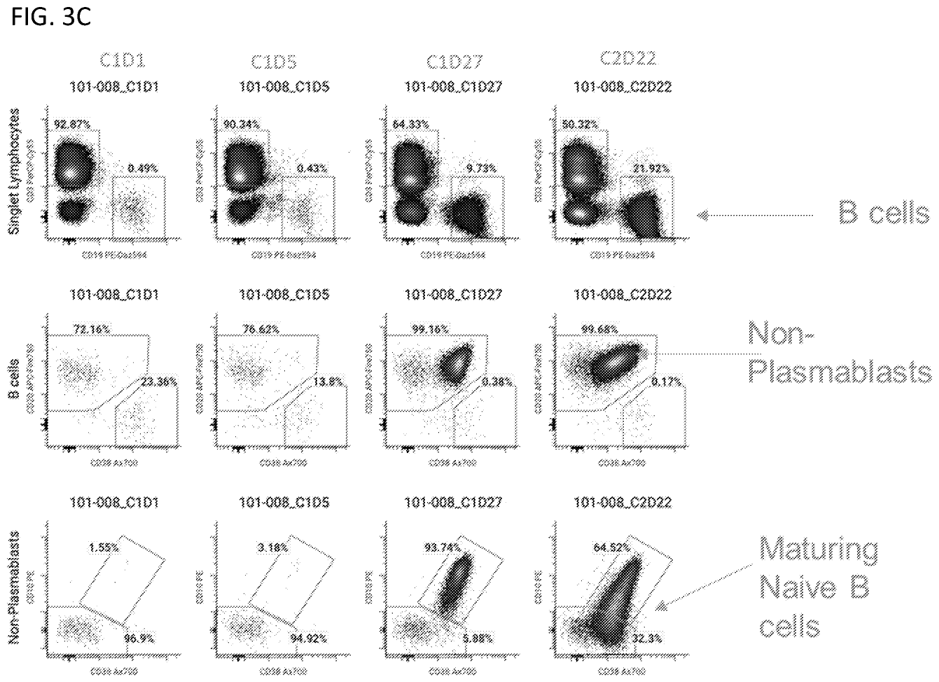

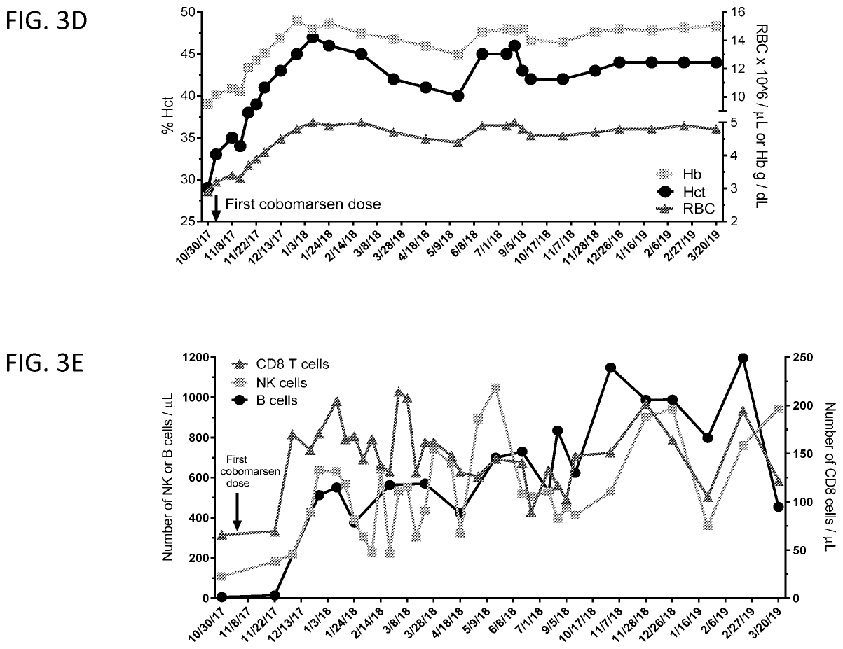

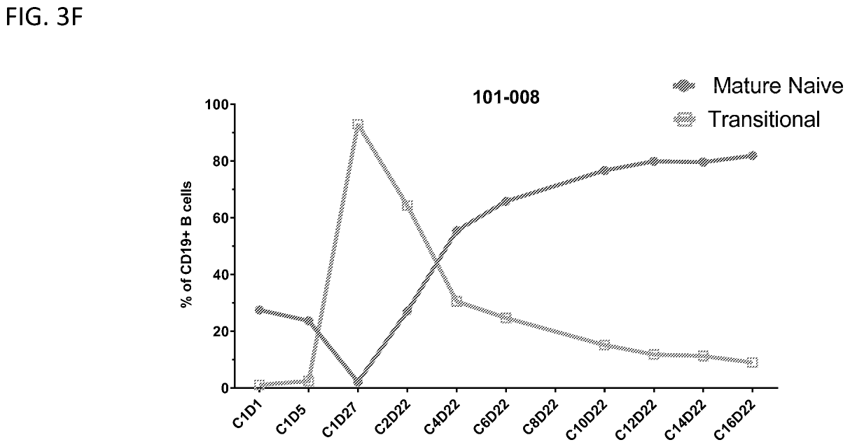

Starting antimiR-155 treatment only 3 weeks after six rounds of chemotherapy did not negatively impact bone marrow recovery and the normalization of peripheral blood immune cell populations in this patient as seen by the recovery of red and white blood cells back to normal ranges. FIGS. 3A and 3B are the as-filed figures from the provisional application and show data from the beginning of the antimiR-155 treatment until the time of the provisional application. In FIG. 3A, decreased RBC counts, hemoglobin (Hb) and hematocrit (Hct) levels measured at screening and on Day 1 of the study before antimir-155 treatment was started, are shown to recover to normal ranges during the first two cycles of therapy. Similar recovery was observed for the CD8 T cell, B cell, and NK cell white blood cell populations over the same time period (FIG. 3B). FIGS. 3D and 3E show the updated data for these parameters from the beginning of the antimiR-155 treatment until the time of the present application (the dates on the X-axis between FIGS. 3A-3B and FIGS. 3D-3E are not identical as these dates are auto-generated by the analysis software).

Assessment of B cell subsets by flow cytometry demonstrated the normal progression of immature transitional B cells (CD10hi, CD38hi) migrating from the bone marrow maturing in to normal naïve B cells (CD10neg, CD38low) in the periphery while on antimir-155 treatment (FIG. 3C). A graphical representation of B cell maturation is shown in FIG. 3F as % mature B cell (CD10⁻CD27⁻IgD⁺) and % transitional B cells (CD10⁺CD38⁺⁺) of CD19⁺ cells over the course of treatment.

Patient No. 101-010 with Lymphomatous ATLL: A 47 year old male patient was diagnosed with lymphomatous ATLL in April 2017 and was treated with the CHOEP chemotherapy for about 5 months. Extensive and bulky lymphadenopathy on initial CT scan was reduced significantly by the CHOEP chemotherapy regimen. At the time of antimiR-155 treatment initiation six weeks post-chemotherapy, the subject had low tumor burden but had not achieved a complete response. A first dose of the antimiR-155 was administered to the patient according to the schedule and the dose noted above. The antimiR-155 treatment has maintained stable lymph node size and peripheral blood tumor cell counts in this patient for over 11 months. The spike in WBC count on Nov. 7, 2018 was attributed primarily to large increase in neutrophils and correlates with a reported rhinovirus infection on Nov. 3, 2018 for this patient. This patient is now off study for progression after completing cycle 18.

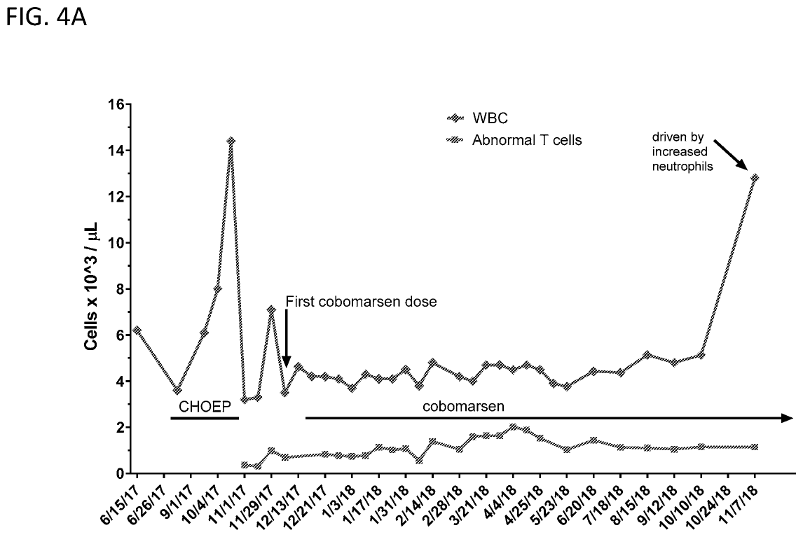

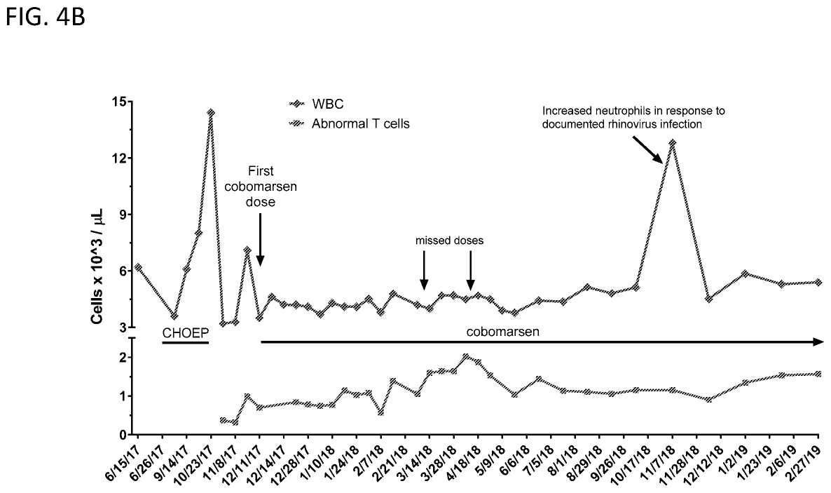

FIG. 4A shows the tumor cell count (abnormal T cells) and the WBC count from the beginning of the treatment until filing of the provisional application. FIG. 4B shows the tumor cell count and the WBC count from the beginning of the treatment until filing of the present application (the dates on the X-axis between FIGS. 4A and 4B are not identical for the reasons explained earlier). Adverse events reported as possibly or definitely related to study drug, at the time the provisional application was filed, included nausea, decreased WBC and platelet count, fatigue, increased AST/ALT, and cough. The adverse events were generally transient and mild in severity (Grade 1). The updated records for this patient at the time of filing the present application include nausea, fatigue, increased AST/ALT, pruritus, and erectile dysfunction.

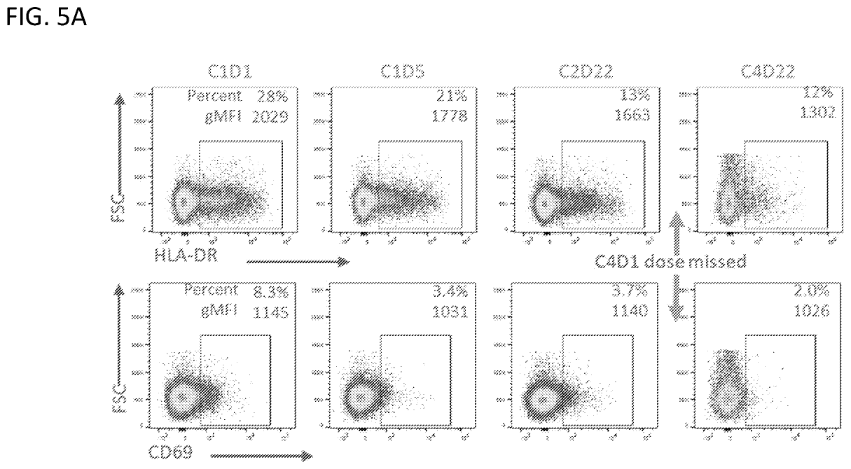

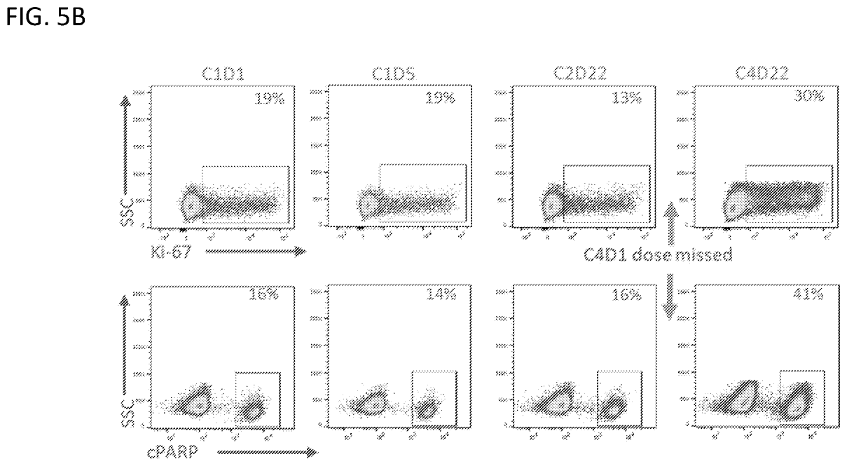

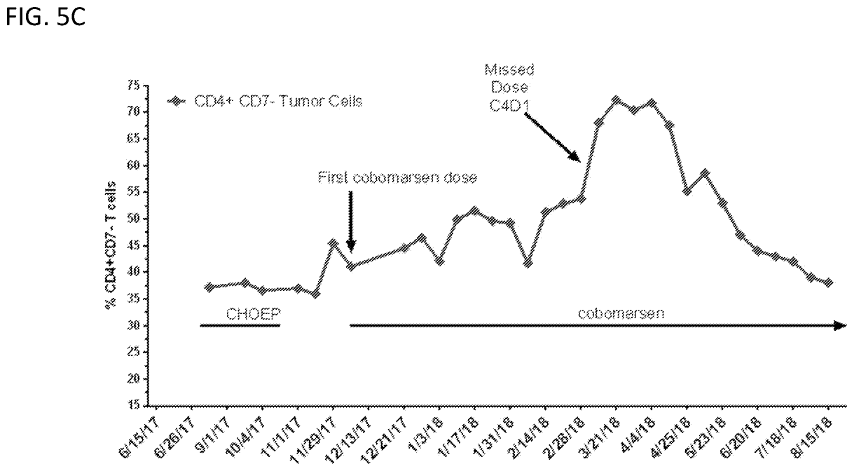

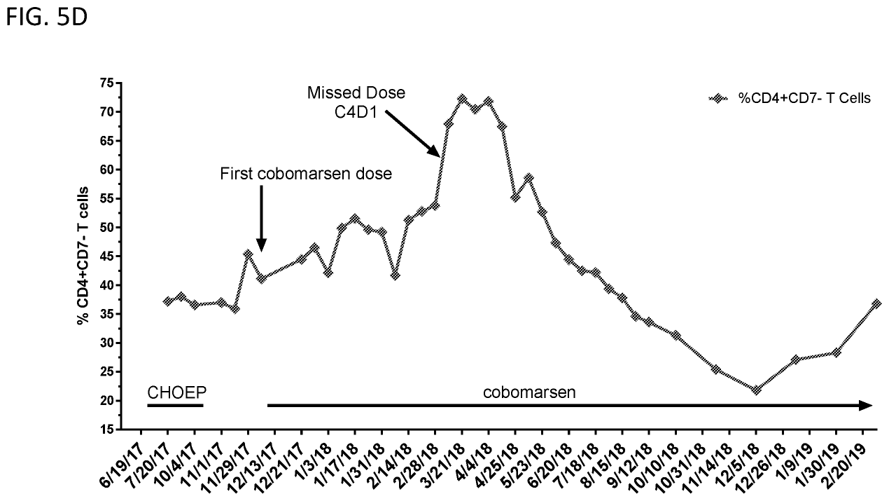

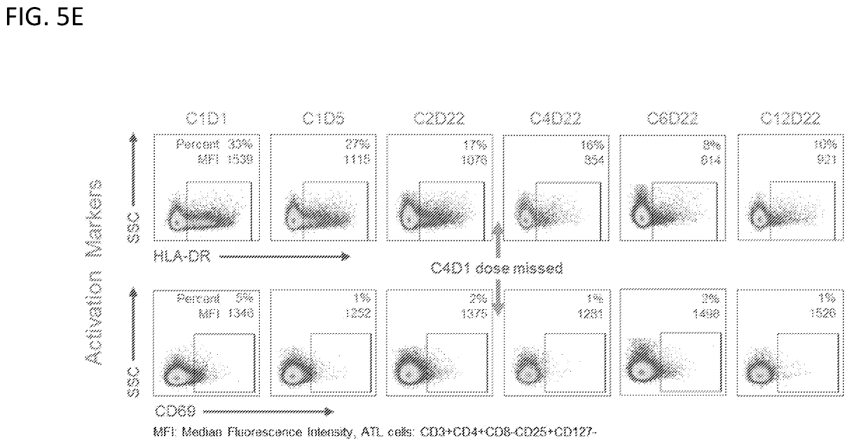

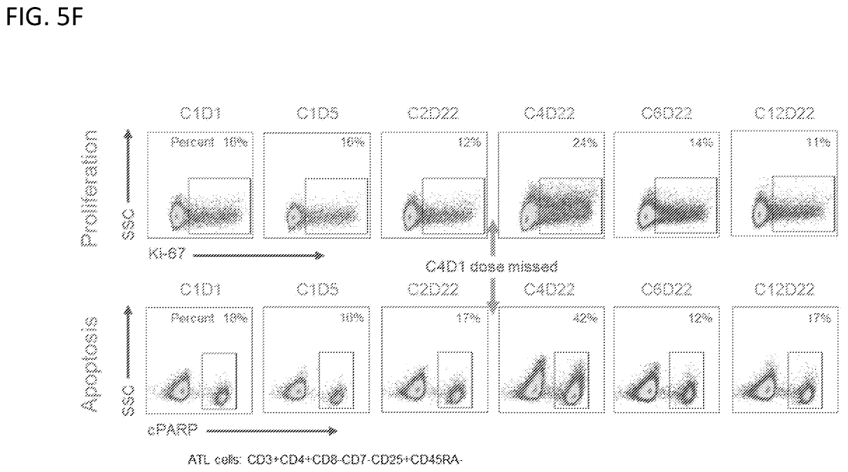

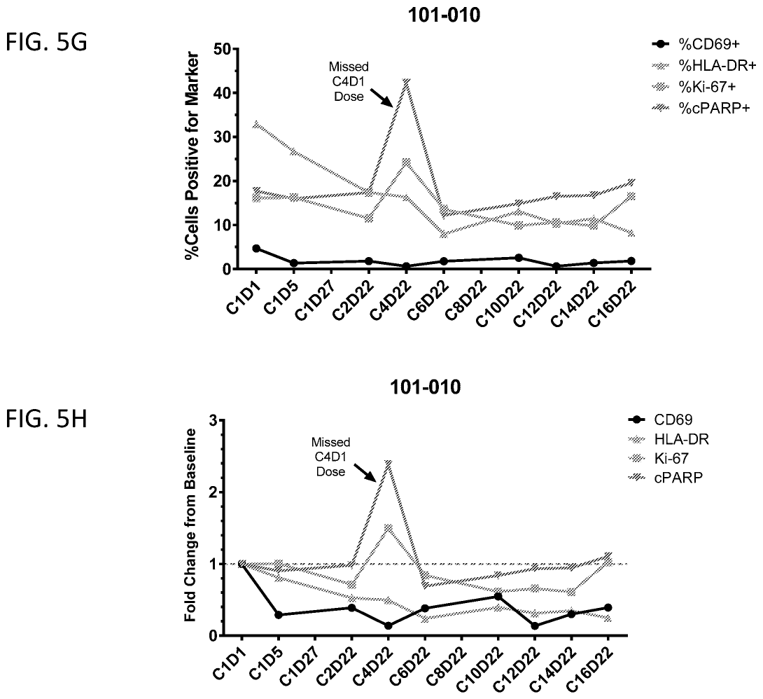

The antimiR-155 treatment decreased the activation and proliferation status of circulating tumor cells in the patient as measured by the mean fluorescent intensity (MFI) and percent tumor cell count for activation markers HLA-DR and CD69 and proliferation marker Ki67 using flow cytometry. FIGS. 5A-5B show the flow cytometry data from the beginning of the antimiR-155 treatment until the filing of the provisional application.

FIG. 5A shows the decreased level (MFI) and decreased percent positive tumor cells as measured by activation markers HLA-DR and CD69 after the anitimiR-155 treatment. Cycle 4 Day 22 (C4D22) data showed an increased percentage of proliferation (Ki67+) or apoptosis (cPARP) markers in tumor cells in periphery following a missed dose on Cycle 4 Day 1 for weather related reasons.

FIG. 5B, upper panel, shows the cell cycle marker Ki67 in tumor cells decreased by the antimiR-155 treatment. FIG. 5B, lower panel, shows the apoptosis marker cPARP in peripheral tumor cells. Cycle 4 Day 22 (C4D22) data showed an increased percentage of proliferation (Ki67+) or apoptosis (cPARP) markers in tumor cells in periphery following a missed dose on Cycle 4 Day 1 (C4D1) for weather related reasons. Percent tumor cells in lymphocyte population increased coincident to increased Ki67 expression but returned to previous C3D22 levels by the end of the next cycle (C5D22).

FIGS. 5E-5F show the flow cytometry data from the beginning of the antimiR-155 treatment until filing of the present application. FIG. 5E shows the status of HLA-DR and CD69 as analyzed at the time of filing of the present application. FIG. 5F shows the status of Ki-67 and cPARP as analyzed at the time of filing of the present application. For the reasons explained earlier (setting of the gates), the percent positive cells and the MFI for the same data points (C1D1, C1D5, C2D22, and C4D22) between FIGS. 5A-5B and 5E-5F do not match although the raw data for these data points is the same.

FIG. 5C shows the percentage of tumor cells from the beginning of the ATLL treatment until the filing of the provisional application. FIG. 5D shows the percentage of tumor cells from the beginning of the ATLL treatment until the filing of the present application. FIG. 5C shows that a missed dose of the antimiR-155 was followed by a minor increase in tumor cells in periphery that returned to previous levels after following a cycle of antimiR-155 treatment.

FIGS. 5G and 5H show the percentage of ATL cells positive for each biomarker (FIG. 5G) and the fold change from baseline (C1D1) (FIG. 5H) over the course of the antimiR-155 treatment.

Patient No. 102-012 with lymphomatous ATLL: A 68 year old male was diagnosed with lymphomatous ATLL in April 2014. The patient manifested extensive skin disease with peripheral ATL cells and was heavily pretreated with Etoposide, AZT/IFN (zidovudine and interferon alfa-2b), Romidepsin, Mogamulizumab, Pralatrexate, lenalidomide, Kenalog, ALR-6924 over the period of about 3.5 years. The skin lesions were treated with PUVA (photochemotherapy with Psoralens (P) and then exposing the skin to UVA (long wave ultraviolet radiation)), Triamcinolone and clobetasol. At the time of antimiR-155 therapy initiation, the patient was considered to have a high tumor burden primarily residing in his skin.

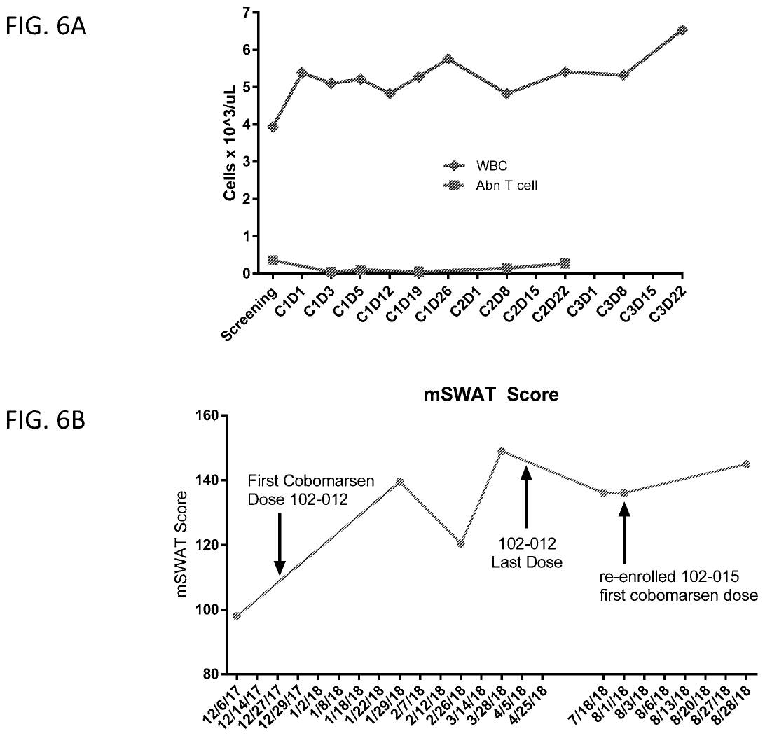

A first dose of the antimiR-155 was administered to the patient according to the schedule and the dose noted above after receiving the above-mentioned therapies. Possibly related adverse events reported included Grade 1 bilateral swelling of hands and feet and Grade 2 bilateral swelling of the thighs. The number of abnormal T cells relative to the white blood cells (WBCs) after the antimiR-155 treatment are shown in FIG. 6A. Due to the patient having primarily skin associated disease, the Modified Severity Weighted Assessment Tool (mSWAT) scores were used to assess the patient's response to drug treatment. mSWAT scores determine the percentage body surface area (BSA) covered by patch, plaque, or tumor of an MF patient, then multiply each lesion BSA by a factor that gives gradations of weight to patch (x1) versus plaque (x2) versus tumor (x4) and sums these scores. After completing 3 cycles of treatment, patient 101-012's (mSWAT) scores had increased over his pretreatment values (FIG. 6B) and his CT scan showed a slight increase in lesion size.

CT scan data for: Nov. 29, 2017 to Apr. 3, 2018:
Lesion 1: 0.9×1.8→1.1×2.0
Lesion 2: 1.1×2.0→1.5×2.2

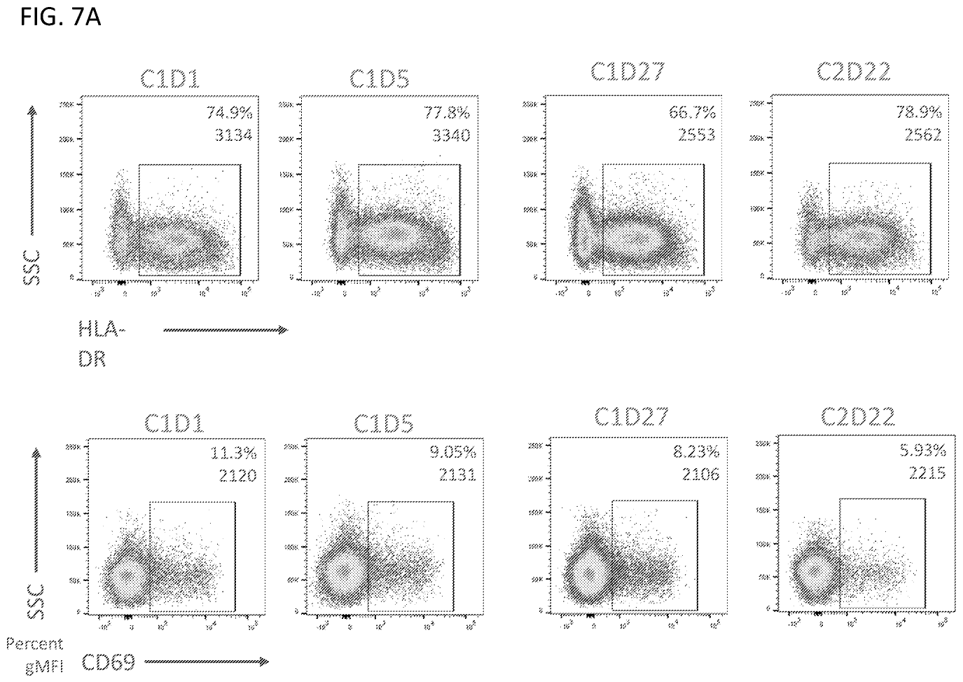

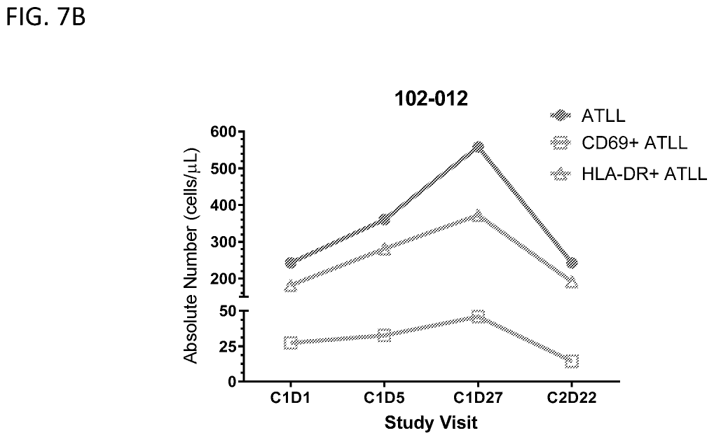

The patient came off study for progression on Apr. 5, 2018. Subsequently, the patient re-enrolled as Patient No. 102-015 due to rapid skin disease progression after stopping the antimiR-155 therapy. However, the patient's mSWAT scores continued to increase and the patient came off study after the 7th dose in the second course of antimiR-155 treatment. The status of activation markers HLA-DR and CD69 on the tumor cells after the antimiR-155 treatment is shown in FIG. 7. FIG. 7C shows the total tumor cell number and the tumor cells expressing CD69 or HLA-DR in the patient.

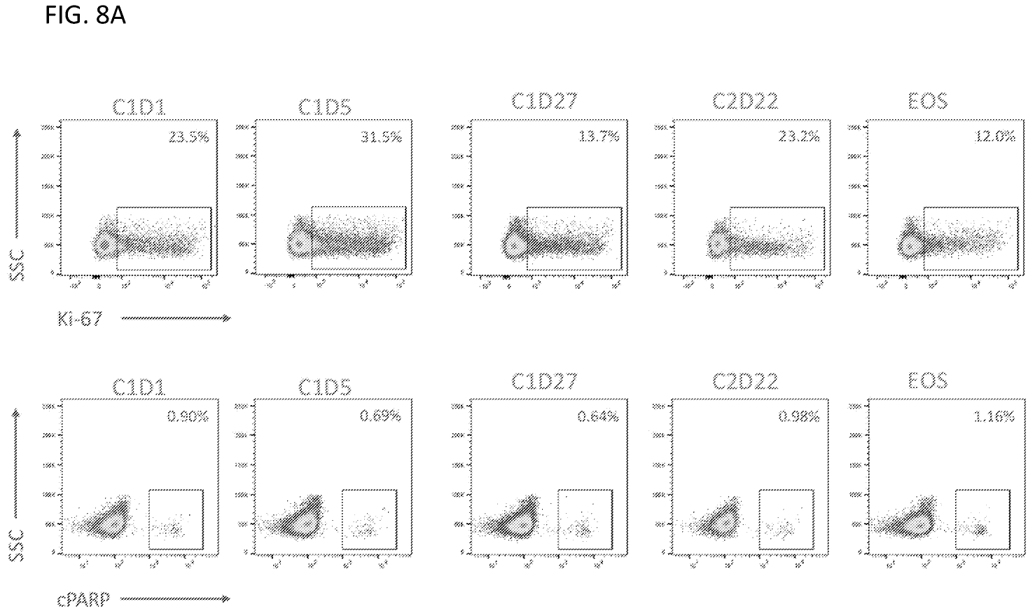

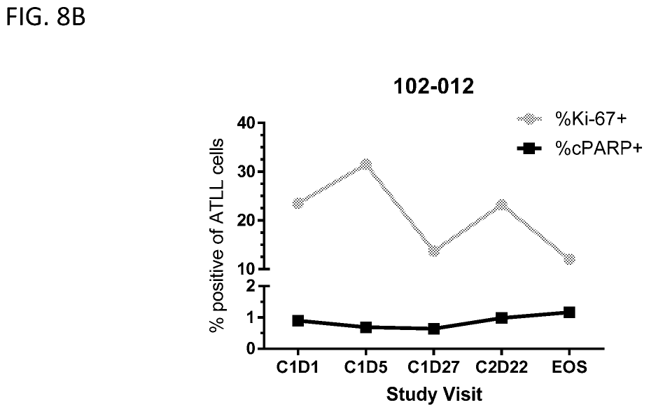

The status of the proliferation marker Ki67 and the apoptosis marker cPARP on the tumor cells is shown in FIG. 8.

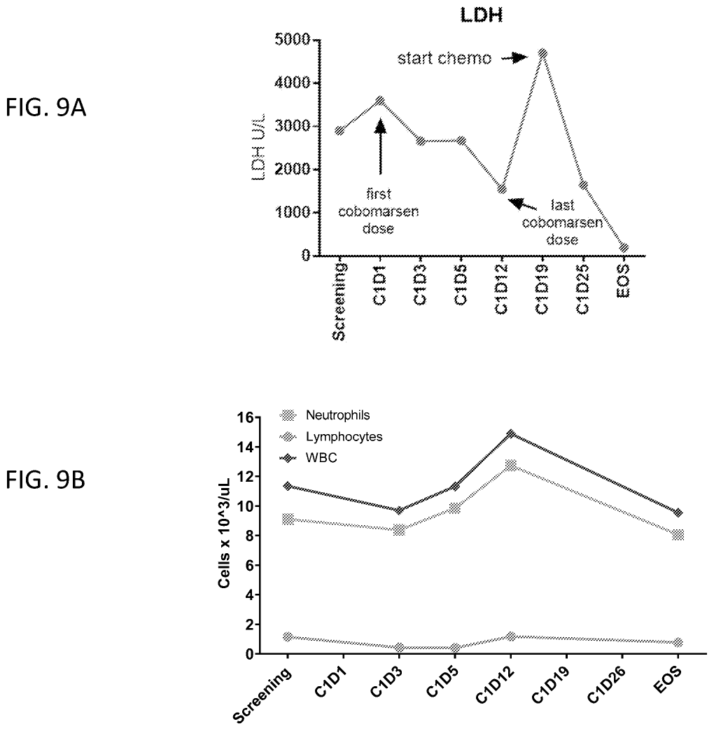

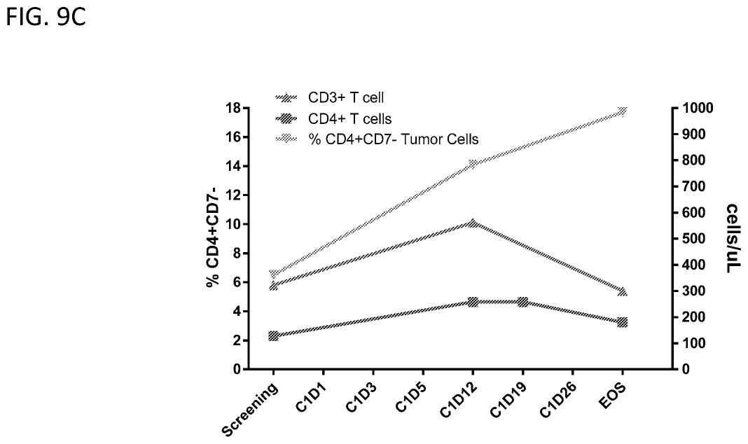

Patient No. 101-011 with lymphomatous ATLL: A 67 year old female was diagnosed with lymphomatous ATLL in June 2016. The patient was pre-treated with high dose methotrexate, EPOCH, and Lenolidomide for ATLL prior to receiving the first dose of antimiR-155. The patient was quickly progressing and had a high tumor burden at the time antimiR-155 was initiated using the schedule and the dose noted above. The patient was treated with the antimiR-155 for 10 days before the treating physician decided to start aggressive chemotherapy. During the short course of antimiR-155 treatment, the patient's LDH levels decreased 57% from 3600 U/L to 1554 U/L (normal range 53-234 U/L) and then rebounded to 4700 U/L after therapy was discontinued (FIG. 9A). FIG. 9B shows the neutrophil, white blood cells, and lymphocyte count in the patient over one cycle of the antimiR-155 treatment. FIG. 9C shows percent CD3⁺, and CD4⁺ T cells, or CD4⁺CD7⁻ tumor cells in the patient over one cycle of the antimiR-155 treatment.

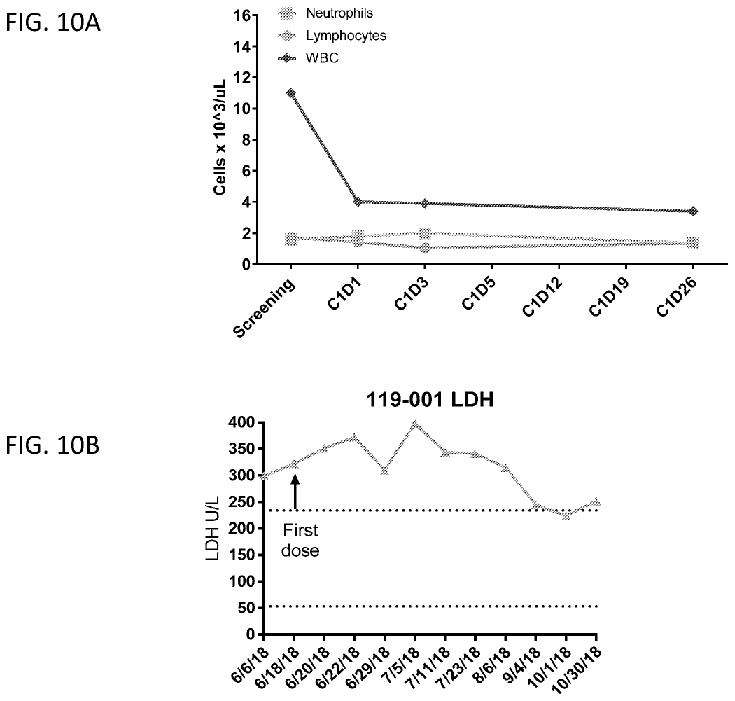

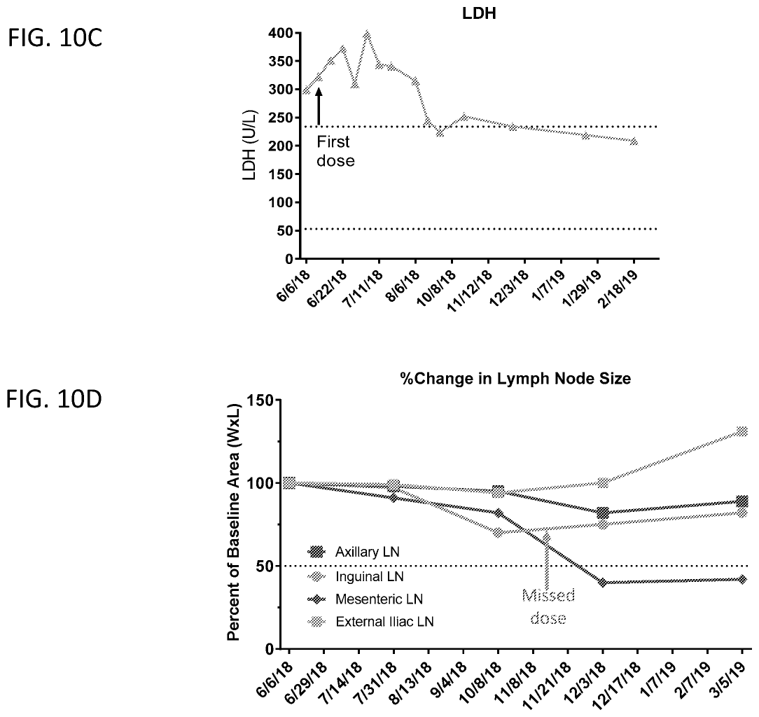

Patient No. 119-001 with lymphomatous ATLL: A 40 year old male was diagnosed with lymphomatous ATLL in June 2013. The patient had previously been treated with VCAP-AMP-VECP, AZT/IFN, gemcitabine with oxaliplatin, belinostat, pralatrexate, IT chemotherapy and AZT/VPA which reduced the tumor burden but did not provide a complete response. The antimiR-155 treatment was initiated from June 2018 according to the schedule and the dose noted above. FIG. 10A shows the neutrophil, white blood cells, and lymphocyte count in the patient over one cycle of the antimiR-155 treatment. FIG. 10B shows the LDH levels from the beginning of the antimiR-155 treatment until filing of the provisional application. FIG. 10C shows the LDH levels from the beginning of the antimiR-155 treatment until filing of the present application. CT scans have shown stable lymph nodes for >5 months with decreasing LDH levels in the over the course of the antimiR-155 treatment. FIG. 10D shows a percent change in the size of 4 lymph nodes over the course of the antimiR-155 treatment. The size refers to area (width×length) as measured by CT scans. The patient is currently receiving the antimiR-155 treatment.

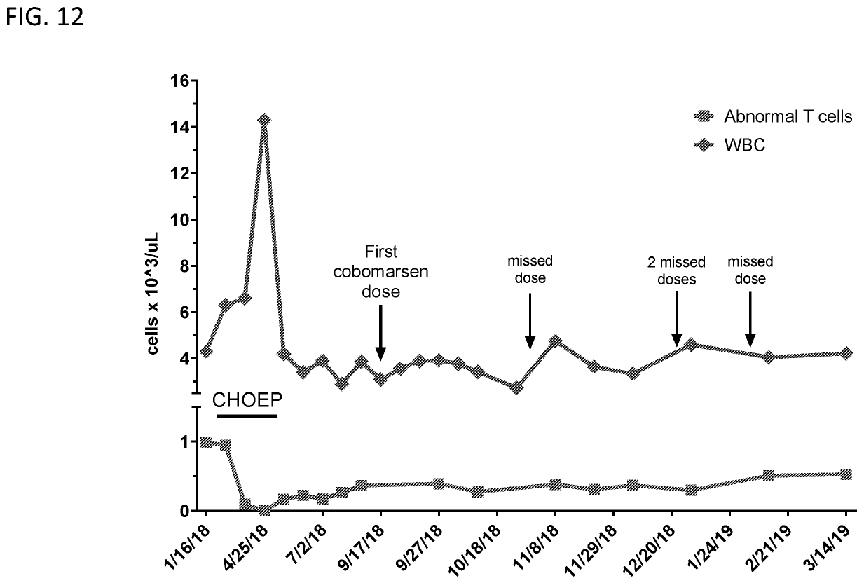

Patient No. 101-012 with acute ATLL: A 52 year old male was diagnosed with acute ATLL in December 2017. The patient had previously been treated with CHOEP chemotherapy resulting in a low tumor burden prior to initiating the antimiR-155 treatment from September, 2018 according to the schedule and the dose noted above. Reported possibly related adverse events, at the time the provisional application was filed, included transient Grade 1 abdominal discomfort and increased AST/ALT and alkaline phosphatase. The updated records for this patient at the time of filing the present application list transient Grade 1 abdominal discomfort, increased AST/ALT, alkaline phosphatase, cough, headache, and dyspnea. The patient is currently stable and continues to receive antimiR-155 treatment. As of this filing, the patient has completed cycle 6 of the antimiR-155 treatment and has missed four doses as shown in the graph in FIG. 12. CT scans of the lymph nodes have remained normal over the course of treatment with the last scan performed in January 2019. LDH values tested for this patient have been within the normal range.

Patient No. 101-014 with lymphomatous ATLL: A 52 year old female was diagnosed with lymphomatous ATLL in December, 2017. The patient had previously been treated with CHOEP chemotherapy and radiation resulting in a low tumor burden prior to receiving the antimiR-155 treatment from September, 2018 according to the schedule and the dose noted above. Reported possibly related adverse events included Grade 1 nausea, vomiting, intermittent diarrhea and worsening anemia. This subject is off study now for progression at the end of Cycle 5.

Patient No. 118-001 with chronic unfavorable ATLL: A 50 year old female was diagnosed with chronic unfavorable ATLL in May, 2016. The patient had high tumor burden of primarily skin disease and had previously been treated with lenalidomide and romidepsin prior to receiving the antimiR-155 treatment from Oct. 1, 2018 according to the schedule and the dose noted above. The patient reported no adverse events. The patient came off study after the first cycle of treatment due to physician's decision on Nov. 16, 2018.

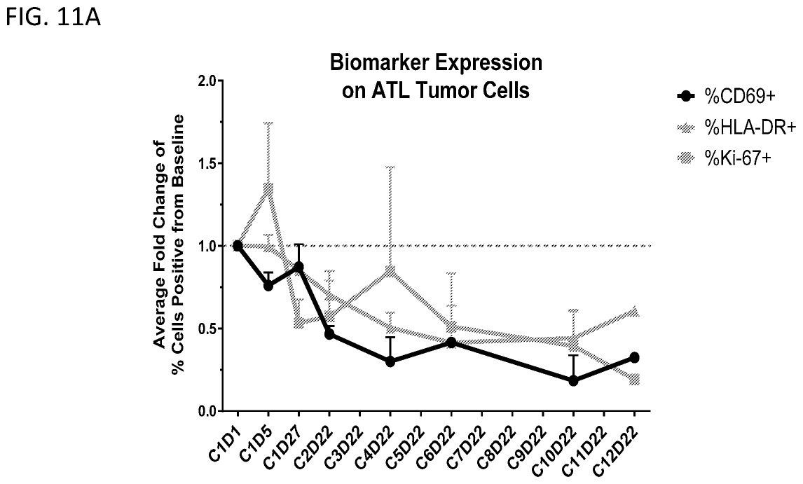

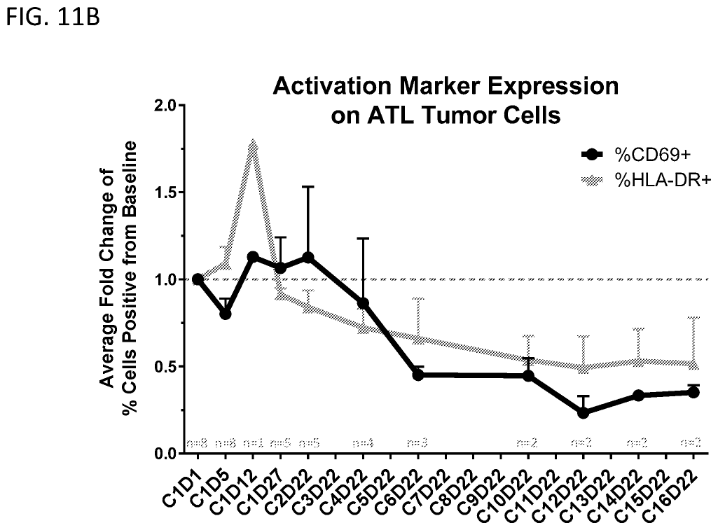

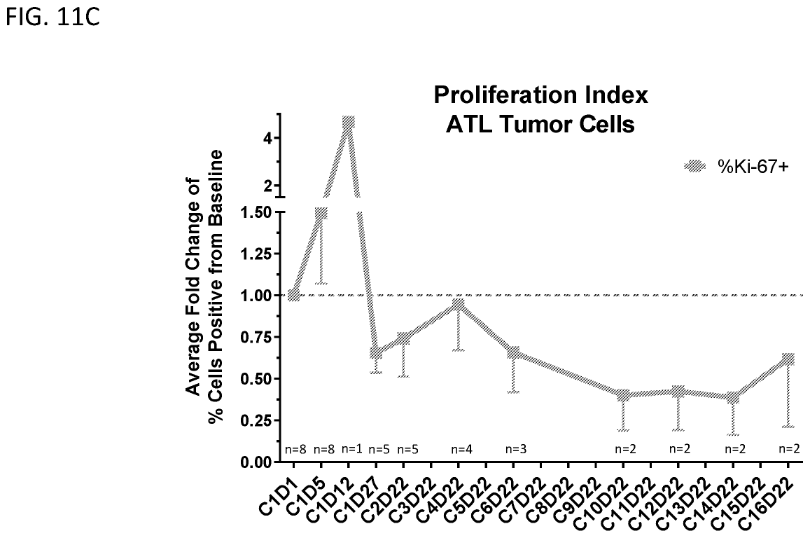

Average expression of certain biomarkers on evaluable patients: All evaluable patients' circulating tumor cells were phenotyped by flow cytometry from peripheral blood samples taken before and during antimiR-155 treatment. On average, following one cycle (28 days) of therapy, circulating tumor cells had decreased activation markers and a lower proliferation index as measured by the mean fluorescent intensity (MFI) and percent tumor cell count for activation markers HLA-DR and CD69 and proliferation marker Ki67. FIG. 11A shows an average fold change (±SEM) from the pretreatment time point (C1D1) in the percentage of ATL tumor cells expressing HLA-DR and CD69 activation markers and Ki-67 proliferation marker from all evaluable ATLL subjects over multiple cycles of the antimiR-155 treatment. The reductions in these activation and proliferation indices was maintained for up to 12 months with continued treatment. In FIG. 11A, the notation C1D1 stands for Cycle 1 Day 1, the notation C1D5 stands for Cycle 1 Day 5, and so forth.

The data in FIG. 11A is from the beginning of the antimiR-155 treatment until filing of the provisional application. FIGS. 11B and 11C show data from the beginning of the antimiR-155 treatment until filing of the present application. Specifically, FIGS. 11B-11C show an average fold change (±SEM) from the pretreatment time point (C1D1) in the percentage of ATL tumor cells expressing HLA-DR and CD69 activation markers (FIG. 11B) and Ki-67 proliferation marker (FIG. 11C) from all evaluable ATLL subjects over multiple cycles of the antimiR-155 treatment. The number of subjects assessed is indicated as n on the X-axis.

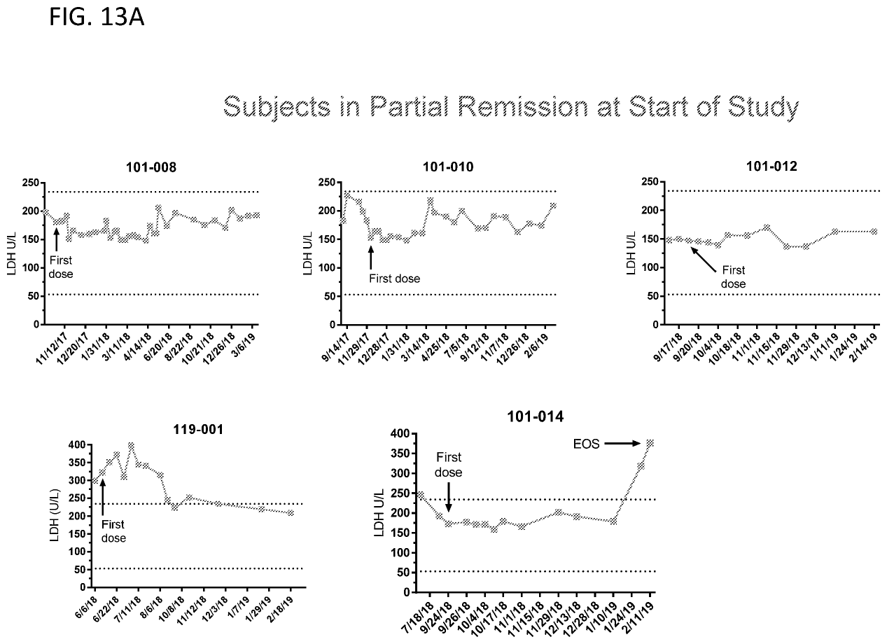

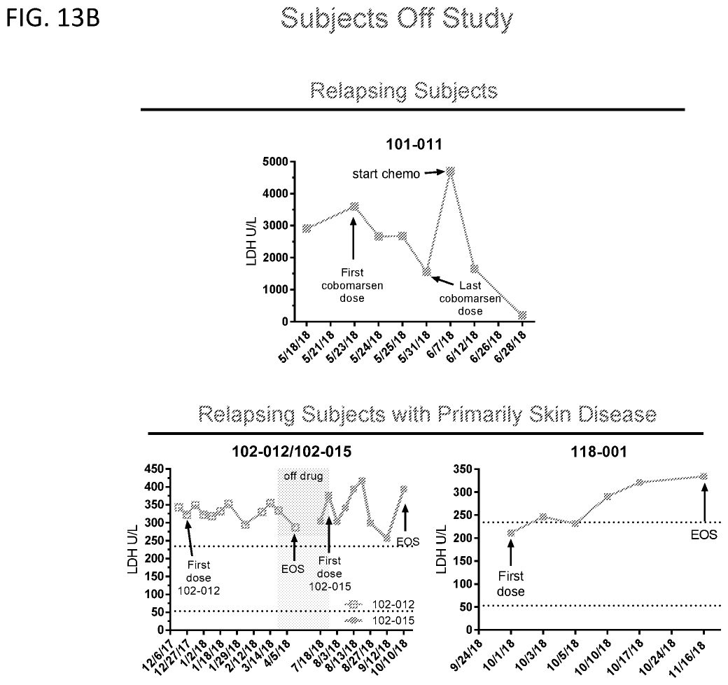

LDH values in ATLL subjects in partial remission and relapsing subjects: FIGS. 13A-13B shows blood LDH values for the patients who were in partial remission at the start of the study versus relapsing patients. Specifically, FIG. 13A shows the LDH levels from the patients that were in partial remission at the start of the study. FIG. 13B shows the LDH levels from the patients that were actively relapsing as they started the study. LDH levels appear to correlate with response and loss of response in these subjects. Based on this, it appears that the blood LDH levels could be used as a marker to assess the effectiveness of an antimiR-155 treatment.

Pharmacokinetic (PK) parameters: A summary of the determined pharmacokinetic parameters for the antimiR-155 upon administration of a single dose and multiple doses of the antimiR-155 intravenously is given in Table 4. The number of subjects assessed for a given PK parameter is indicated as N.

TABLE 4

| | | | Tₘₐₓ* | Cₘₐₓ | AUC₀₋₂₄ | AUC_{inf} |
|---|---|---|---|---|---|---|
| | Dose | | (hr) | (μg/mL) | (μg*hr/mL) | (μg*hr/mL) |
| Single | 600 mg | N | 8 | 8 | 7 | 7 |
| Dose | | Mean | 1.92 | 22.9 | 78.6 | 81.8 |
| | | SD | | 6.08 | 22.3 | 24.1 |
| Multiple | 600 mg | N | 4 | 4 | 2 | 2 |
| Dose | | Mean | 1.92 | 17.3 | 75.9 | 80.9 |
| | | SD | | 2.85 | 25.6 | 29.4 |

*Median data reported for Tₘₐₓ, which occurred at the End of Infusion

### Numbered Embodiments of the Invention

1. A method for treating Adult T-cell Leukemia/Lymphoma (ATLL) in a subject in need thereof, comprising:
   a. administering to the subject one or more therapeutic agents for treating ATLL;
   b. administering an oligonucleotide inhibitor of miR-155 to the subject after the administration of the one or more therapeutic agents.
2. A method for decreasing or maintaining the tumor cell count in a subject suffering from Adult T-cell Leukemia/Lymphoma (ATLL), comprising administering an oligonucleotide inhibitor of miR-155 to the subject, wherein the subject has received one or more therapeutic agents for treating ATLL prior to the treatment with the oligonucleotide inhibitor of miR-155.
3. A method for treating a disease associated with a Human T-cell Lymphotropic Virus (HTLV) in a subject in need thereof, comprising administering to the subject an oligonucleotide inhibitor of miR-155.
4. The method of embodiment 3, wherein the HTLV is HTLV-1, HTLV-2, HTLV-3, and/or HTLV-4.
5. The method of embodiment 3 or 4, wherein the disease associated with the HTLV is Adult T-cell Leukemia/Lymphoma (ATLL), HTLV-1 associated myelopathy/tropical spastic paraparesis (HAM/TSP), or HTLV-associated dermatitis.
6. The method of any one of embodiments 3-5, wherein the disease associated with the HTLV is ATLL.
7. The method of any one of embodiments 1, 2, 5, and 6, wherein the ATLL is Leukemic ATLL or Lymphomatous ATLL.
8. The method of embodiment 7, wherein the Leukemic ATLL is Acute Leukemic ATLL.
9. The method of any one of embodiments 6-8, comprising administering to the subject one or more therapeutic agents for treating ATLL prior to or after the administration of the oligonucleotide inhibitor of miR-155.
10. The method of any one of embodiments 1, 2 and 9, wherein the one or more therapeutic agents are selected from the group consisting of zidovudine, interferon alpha, lenalidomide, etoposide, prednisone, vincristine, cyclophosphamide, doxorubicin hydrochloride, rituximab, romidepsin, mogamulizumab, pralatrexate, triamcinolone acetonide, ranimustine, vindesine, carboplatin, ALRN-6924 (a stapled alpha-helical peptide), methotrexate, gemcitabine, oxaliplatin, belinostat, acyclovir, Histone deacetylase (HDAC) inhibitors (HDIs), radiation therapy, and combinations thereof
11. The method of any one of embodiments 1, 2, 9, and 10, wherein the one or more therapeutic agents are zidovudine, interferon alpha, lenalidomide, etoposide, prednisone, vincristine, cyclophosphamide, and doxorubicin hydrochloride.
12. The method of any one of embodiments 1, 2, 9, and 10, wherein the one or more therapeutic agents are cyclophosphamide, doxorubicin, vincristine, etoposide, and prednisone.
13. The method of any one of embodiments 1, 2, 9, and 10, wherein the one or more therapeutic agents are etoposide, zidovudine, interferon alpha, romidepsin, mogamulizumab, pralatrexate, lenalidomide, triamcinolone acetonide, and ALRN-6924.
14. The method of any one of embodiments 1, 2, and 6-13, wherein the subject is receiving or has received a photochemotherapy.
15. The method of embodiment 14, wherein the photochemotherapy is a combination treatment comprising psoralens and long wave ultraviolet radiation.
16. The method of any one of embodiments 1, 2, and 6-13, wherein the one or more therapeutic agents for treating ATLL are administered to the subject at least for one cycle prior to the administration of the oligonucleotide inhibitor of miR-155.
17. The method of any one of embodiments 1-16, wherein the oligonucleotide inhibitor of miR-155 is administered according to a cycle, said cycle comprising:
   c. first administering at least one loading dose; and
   d. second administering at least one maintenance dose.
18. The method of embodiment 17, wherein the loading dose is about 2 times, 3 times, 4 times, or 5 times the maintenance dose.
19. The method of embodiment 17 or 18, wherein the loading dose is about 1500 mg to about 2400 mg.
20. The method of any one of embodiments 17-19, wherein the loading dose is about 1500 mg, 1800 mg, 2100 mg, or about 2400 mg.
21. The method of any one of embodiments 17-20, wherein the loading dose is about 1800 mg.
22. The method of any one of embodiments 17-21, wherein the loading dose is split into three doses administered every other day.
23. The method of any one of embodiments 17-22, wherein the loading dose is about 1800 mg split into three doses with each split dose being about 600 mg administered every other day.
24. The method of any one of embodiments 17-22, wherein the loading dose is about 1800 mg split into three doses of about 600 mg each administered on day 1, 3, and 5 of the cycle.
25. The method of any one of embodiments 17-24, wherein the maintenance dose is about 400 mg to about 1200 mg.
26. The method of any one of embodiments 17-25, wherein the maintenance dose is about 400 mg, 600 mg, 800 mg, 1000 mg, or about 1200 mg.
27. The method of any one of embodiments 17-26, wherein the maintenance dose is about 600 mg.
28. The method of any one of embodiments 17-27, wherein the maintenance dose is administered once a week.
29. The method of any one of embodiments 17-28, wherein the maintenance dose is about 600 mg administered once a week.
30. The method of any one of embodiments 1-29, wherein the oligonucleotide inhibitor of miR-155 comprises the sequence selected from Table 2.
31. The method of any one of embodiments 1-30, wherein the oligonucleotide inhibitor of miR-155 comprises the sequence of the sequence of 5′-lCs.dAs.lCs.dGs.dAs.lTs.lTs.dAs.lGs.dCs.lAs.lTs.lTs.lA-3′.
32. The method of any one of embodiments 1, 2, and 6-31, wherein the administration of the oligonucleotide inhibitor of miR-155 decreases the tumor cell count compared to the tumor cell count at the beginning of the administration of the oligonucleotide inhibitor of miR-155.
33. The method of any one of embodiments 1, 2, and 6-32, wherein the administration of the oligonucleotide inhibitor of miR-155 decreases the tumor cell count compared to the tumor cell count at the beginning of the administration of the oligonucleotide inhibitor of miR-155 and maintains the tumor cell count at or below the decreased level.
34. The method of any one of embodiments 1, 2, and 6-33, wherein the administration of the oligonucleotide inhibitor of miR-155 decreases the tumor cell count by about 10 to 80% compared to the tumor cell count at the beginning of the administration of the oligonucleotide inhibitor of miR-155.
35. The method of any one of embodiments 1, 2, and 6-31, wherein the administration of the oligonucleotide inhibitor of miR-155 maintains the tumor cell count at or below the level that was present at the beginning of the administration of the oligonucleotide inhibitor of miR-155.
36. The method of any one of embodiments 1, 2, and 6-35, wherein the administration of the oligonucleotide inhibitor of miR-155 decreases the size of enlarged lymph nodes of the subject.
37. The method of any one of embodiments 1, 2, and 6-36, wherein the administration of the oligonucleotide inhibitor of miR-155 decreases the size of enlarged lymph nodes of the subject to a normal size.
38. The method of any one of embodiments 1, 2, and 6-36, wherein the administration of the one or more chemotherapeutic agents decreases the size of lymph nodes in the subject and the administration of the oligonucleotide inhibitor of miR-155 maintains the size of lymph nodes to the decreased size.
39. The method of any one of embodiments 1, 2, and 6-38, wherein the administration of the oligonucleotide inhibitor of miR-155 decreases the activation and/or proliferation of tumor cells.
40. The method of any one of embodiments 1, 2, and 6-39, wherein the administration of the oligonucleotide inhibitor of miR-155 decreases the activation of tumor cells as measured by the levels of activation markers HLA-DR and/or CD69.
41. The method of any one of embodiments 1, 2, and 6-40, wherein the administration of the oligonucleotide inhibitor of miR-155 decreases the levels of activation markers HLA-DR and/or CD69 on tumor cells by about 5-80% compared to the levels at the beginning of the administration of the oligonucleotide inhibitor of miR-155.
42. The method of any one of embodiments 1, 2, and 6-41, wherein the administration of the oligonucleotide inhibitor of miR-155 decreases the proliferation of tumor cells as measured by the levels of cell cycle marker Ki67.
43. The method of any one of embodiments 1, 2, and 6-42, wherein the administration of the oligonucleotide inhibitor of miR-155 decreases the levels of cell cycle marker Ki67 on tumor cells by about 5-80% compared to the levels at the beginning of the administration of the oligonucleotide inhibitor of miR-155.
44. The method of any one of embodiments 1, 2, and 6-43, wherein the administration of the oligonucleotide inhibitor of miR-155 decreases the proliferation of tumor cells by about 5-90% compared to the proliferation of tumor cells at the beginning of the administration of the oligonucleotide inhibitor of miR-155.
45. The method of any one of embodiments 1-44, wherein the oligonucleotide inhibitor of miR-155 is administered for about 6 week, 2 months, 3 months, 4 months, 5 months, 6 months, 8 months, 9 months, 10 months, or 12 months.
46. The method of any one of embodiments 1, 2, and 6-32, wherein the administration of the oligonucleotide inhibitor of miR-155 decreases the tumor cell count compared to the tumor cell count at the beginning of the administration of the oligonucleotide inhibitor of miR-155 and maintains the tumor cell count to the decreased level for 6 months or more.
47. The method of any one of embodiments 1, 2, and 6-31, wherein the administration of the oligonucleotide inhibitor of miR-155 maintains the tumor cell count to the level that was present at the beginning of the administration of the oligonucleotide inhibitor of miR-155 for 6 months or more.
48. The method of any one of embodiments 1-47, wherein the administration of the oligonucleotide inhibitor of miR-155 increases the progression-free survival of the subject by at least about 3 months, 4 months, 6 months, 8 months, 10 months, 12 months, 15 months, 18 months, 24 months or more.
49. The method of any one of embodiments 3-48, wherein the administration of the oligonucleotide inhibitor of miR-155 reduces the HTLV load in the subject.
50. The method of any one of embodiments 3-49, wherein the administration of the oligonucleotide inhibitor of miR-155 reduces the HTLV load in the subject by about 10-80%.
51. The method of any one of embodiments 1-50, wherein the administration of the oligonucleotide inhibitor of miR-155 promotes restoration of normal homeostatic immune response in the subject.
52. The method of any one of embodiments 1-51, wherein the administration of the oligonucleotide inhibitor of miR-155 promotes re-population of healthy B and/or T cells in the subject.
53. The method of any one of embodiments 1-52, wherein the administration of the oligonucleotide inhibitor of miR-155 increases the number of healthy B and/or T cells in the subject by about 5 to 60%.
54. A method for treating Adult T-cell Leukemia/Lymphoma (ATLL) in a subject in need thereof, comprising administering to the subject an oligonucleotide inhibitor of miR-155, wherein the oligonucleotide inhibitor of miR-155 decreases or maintains the tumor cell count in the subject.
55. The method of any one of embodiments 1, 2, 5, 6, and 54, wherein the ATLL is Leukemic ATLL, Chronic ATLL, Smouldering ATLL, or Lymphomatous ATLL.
56. The method of embodiment 55, wherein the Leukemic ATLL is Acute Leukemic ATLL.
57. The method of embodiment 55, wherein the Lymphomatous ATLL is Acute Lymphomatous ATLL.
58. The method of any one of embodiments 54-57, comprising administering to the subject one or more therapeutic agents for treating ATLL prior to or after the administration of the oligonucleotide inhibitor of miR-155.
59. The method of embodiment 58, wherein the one or more therapeutic agents are selected from the group consisting of zidovudine, interferon alpha, lenalidomide, etoposide, prednisone, vincristine, cyclophosphamide, doxorubicin hydrochloride, rituximab, romidepsin, mogamulizumab, pralatrexate, triamcinolone acetonide, ranimustine, vindesine, carboplatin, ALRN-6924 (a stapled alpha-helical peptide), methotrexate, gemcitabine, oxaliplatin, belinostat, acyclovir, Histone deacetylase (HDAC) inhibitors (HDIs), radiation therapy, and combinations thereof
60. The method of embodiment 58 or 59, wherein the one or more therapeutic agents are zidovudine, interferon alpha, lenalidomide, etoposide, prednisone, vincristine, cyclophosphamide, and doxorubicin hydrochloride.
61. The method of embodiment 58 or 59, wherein the one or more therapeutic agents are cyclophosphamide, doxorubicin, vincristine, etoposide, and prednisone.
62. The method of embodiment 58 or 59, wherein the one or more therapeutic agents are etoposide, zidovudine, interferon alpha, romidepsin, mogamulizumab, pralatrexate, lenalidomide, triamcinolone acetonide, and ALRN-6924.
63. The method of any one of embodiments 54-62, wherein the subject is receiving or has received a photochemotherapy.
64. The method of embodiment 63, wherein the photochemotherapy is a combination treatment comprising psoralens and long wave ultraviolet radiation.
65. The method of any one of embodiments 58-64, wherein the one or more therapeutic agents for treating ATLL are administered to the subject at least for one cycle prior to the administration of the oligonucleotide inhibitor of miR-155.
66. The method of any one of embodiments 54-65, wherein the oligonucleotide inhibitor of miR-155 is administered according to a cycle, said cycle comprising:
   a. first administering at least one loading dose; and
   b. second administering at least one maintenance dose.
67. The method of embodiment 66, wherein the loading dose is about 2 times, 3 times, 4 times, or 5 times the maintenance dose.
68. The method of embodiment 66 or 67, wherein the loading dose is about 1500 mg to about 2400 mg.
69. The method of any one of embodiments 66-68, wherein the loading dose is about 1500 mg, 1800 mg, 2100 mg, or about 2400 mg.
70. The method of any one of embodiments 66-69, wherein the loading dose is about 1800 mg.
71. The method of any one of embodiments 66-70, wherein the loading dose is split into three doses administered every other day.
72. The method of any one of embodiments 66-71, wherein the loading dose is about 1800 mg split into three doses with each split dose being about 600 mg administered every other day.
73. The method of any one of embodiments 66-72, wherein the loading dose is about 1800 mg split into three doses of about 600 mg each administered on day 1, 3, and 5 of the cycle.
74. The method of any one of embodiments 66-73, wherein the maintenance dose is about 400 mg to about 1200 mg.
75. The method of any one of embodiments 66-74, wherein the maintenance dose is about 400 mg, 600 mg, 800 mg, 1000 mg, or about 1200 mg.
76. The method of any one of embodiments 66-75, wherein the maintenance dose is about 600 mg.
77. The method of any one of embodiments 66-76, wherein the maintenance dose is administered once a week.
78. The method of any one of embodiments 66-77, wherein the maintenance dose is about 600 mg administered once a week.
79. The method of any one of embodiments 54-78, wherein the oligonucleotide inhibitor of miR-155 comprises a sequence selected from Table 2.
80. The method of any one of embodiments 54-79, wherein the oligonucleotide inhibitor of miR-155 comprises the sequence of 5′-lCs.dAs.lCs.dGs.dAs.lTs.lTs.dAs.lGs.dCs.lAs.lTs.lTs.lA-3′ (SEQ ID NO: 25).
81. The method of any one of embodiments 54-80, wherein the administration of the oligonucleotide inhibitor of miR-155 decreases the tumor cell count compared to the tumor cell count at the beginning of the administration of the oligonucleotide inhibitor of miR-155.
82. The method of any one of embodiments 54-81, wherein the administration of the oligonucleotide inhibitor of miR-155 decreases the tumor cell count compared to the tumor cell count at the beginning of the administration of the oligonucleotide inhibitor of miR-155 and maintains the tumor cell count at or below the decreased level.
83. The method of any one of embodiments 54-82, wherein the administration of the oligonucleotide inhibitor of miR-155 decreases the tumor cell count by about 10 to 80% compared to the tumor cell count at the beginning of the administration of the oligonucleotide inhibitor of miR-155.
84. The method of any one of embodiments 54-83, wherein the administration of the oligonucleotide inhibitor of miR-155 maintains the tumor cell count at or below the level that was present at the beginning of the administration of the oligonucleotide inhibitor of miR-155.
85. The method of any one of embodiments 54-84, wherein the administration of the oligonucleotide inhibitor of miR-155 decreases the size of enlarged lymph nodes of the subject.
86. The method of any one of embodiments 54-85, wherein the administration of the oligonucleotide inhibitor of miR-155 decreases the size of enlarged lymph nodes of the subject to a normal size.
87. The method of any one of embodiments 58-86, wherein the administration of the one or more chemotherapeutic agents decreases the size of lymph nodes in the subject and the administration of the oligonucleotide inhibitor of miR-155 maintains the size of lymph nodes to the decreased size.
88. The method of any one of embodiments 54-87, wherein the administration of the oligonucleotide inhibitor of miR-155 decreases the activation and/or proliferation of tumor cells.
89. The method of any one of embodiments 54-88, wherein the administration of the oligonucleotide inhibitor of miR-155 decreases the activation of tumor cells as measured by the levels of activation markers HLA-DR and/or CD69.
90. The method of any one of embodiments 54-89, wherein the administration of the oligonucleotide inhibitor of miR-155 decreases the levels of activation markers HLA-DR and/or CD69 on tumor cells by about 5-80% compared to the levels at the beginning of the administration of the oligonucleotide inhibitor of miR-155.
91. The method of any one of embodiments 54-90, wherein the administration of the oligonucleotide inhibitor of miR-155 decreases the proliferation of tumor cells as measured by the levels of cell cycle marker Ki67.
92. The method of any one of embodiments 54-91, wherein the administration of the oligonucleotide inhibitor of miR-155 decreases the levels of cell cycle marker Ki67 on tumor cells by about 5-80% compared to the levels at the beginning of the administration of the oligonucleotide inhibitor of miR-155.
93. The method of any one of embodiments 54-92, wherein the administration of the oligonucleotide inhibitor of miR-155 decreases the proliferation of tumor cells by about 5-90% compared to the proliferation of tumor cells at the beginning of the administration of the oligonucleotide inhibitor of miR-155.
94. The method of any one of embodiments 54-93, wherein the oligonucleotide inhibitor of miR-155 is administered for about 6 week, 2 months, 3 months, 4 months, 5 months, 6 months, 8 months, 9 months, 10 months, or 12 months.
95. The method of any one of embodiments 54-94, wherein the administration of the oligonucleotide inhibitor of miR-155 decreases the tumor cell count compared to the tumor cell count at the beginning of the administration of the oligonucleotide inhibitor of miR-155 and maintains the tumor cell count to the decreased level for 6 months or more.
96. The method of any one of embodiments 54-95, wherein the administration of the oligonucleotide inhibitor of miR-155 maintains the tumor cell count to the level that was present at the beginning of the administration of the oligonucleotide inhibitor of miR-155 for 6 months or more.
97. The method of any one of embodiments 54-96, wherein the administration of the oligonucleotide inhibitor of miR-155 increases the progression-free survival of the subject by at least about 3 months, 4 months, 6 months, 8 months, 10 months, 12 months, 15 months, 18 months, 24 months or more.
98. The method of any one of embodiments 54-97, wherein the administration of the oligonucleotide inhibitor of miR-155 reduces the HTLV load in the subject.
99. The method of any one of embodiments 54-98, wherein the administration of the oligonucleotide inhibitor of miR-155 reduces the HTLV load in the subject by about 10-80%.
100. The method of any one of embodiments 54-99, wherein the administration of the oligonucleotide inhibitor of miR-155 promotes restoration of normal homeostatic immune response in the subject.
101. The method of any one of embodiments 54-100, wherein the administration of the oligonucleotide inhibitor of miR-155 promotes re-population of healthy B and/or T cells in the subject.
102. The method of any one of embodiments 54-101, wherein the administration of the oligonucleotide inhibitor of miR-155 increases the number of healthy B and/or T cells in the subject by about 5 to 60%.
103. A method for treating Adult T-cell Leukemia/Lymphoma (ATLL) in a subject in need thereof, comprising:
   a. administering to the subject one or more therapeutic agents for treating ATLL;
   b. administering an oligonucleotide inhibitor of miR-155 to the subject after the administration of the one or more therapeutic agents, wherein the oligonucleotide inhibitor of miR-155 comprises the sequence of 5′-lCs.dAs.lCs.dGs.dAs.lTs.lTs.dAs.lGs.dCs.lAs.lTs.lTs.lA-3′ (SEQ ID NO: 25).
104. A method for decreasing or maintaining the tumor cell count in a subject suffering from Adult T-cell Leukemia/Lymphoma (ATLL), comprising administering an oligonucleotide inhibitor of miR-155 to the subject, wherein the subject has received one or more therapeutic agents for treating ATLL prior to the treatment with the oligonucleotide inhibitor of miR-155, and wherein the oligonucleotide inhibitor of miR-155 comprises the sequence of 5′-lCs.dAs.lCs.dGs.dAs.lTs.lTs.dAs.lGs.dCs.lAs.lTs.lTs.lA-3′ (SEQ ID NO: 25).
105. A method for treating Adult T-cell Leukemia/Lymphoma (ATLL) in a subject in need thereof, comprising administering to the subject an oligonucleotide inhibitor of miR-155 comprising the sequence of 5′-lCs.dAs.lCs.dGs.dAs.lTs.lTs.dAs.lGs.dCs.lAs.lTs.lTs.lA-3′ (SEQ ID NO: 25), wherein the oligonucleotide inhibitor of miR-155 decreases or maintains the tumor cell count in the subject.
106. A method for treating a disease associated with a Human T-cell Lymphotropic Virus (HTLV) in a subject in need thereof, comprising administering to the subject an oligonucleotide inhibitor of miR-155 comprising the sequence of 5′-lCs.dAs.lCs.dGs.dAs.lTs.lTs.dAs.lGs.dCs.lAs.lTs.lTs.lA-3′ (SEQ ID NO: 25).
107. A method for treating Adult T-cell Leukemia/Lymphoma (ATLL) in a subject in need thereof, comprising:
   a. administering to the subject one or more therapeutic agents for treating ATLL;
   b. administering an oligonucleotide inhibitor of miR-155 to the subject after the administration of the one or more therapeutic agents, wherein the oligonucleotide inhibitor of miR-155 comprises a sequence selected from Table 2.
108. A method for decreasing or maintaining the tumor cell count in a subject suffering from Adult T-cell Leukemia/Lymphoma (ATLL), comprising administering an oligonucleotide inhibitor of miR-155 to the subject, wherein the subject has received one or more therapeutic agents for treating ATLL prior to the treatment with the oligonucleotide inhibitor of miR-155, and wherein the oligonucleotide inhibitor of miR-155 comprises a sequence selected from Table 2.
109. A method for treating Adult T-cell Leukemia/Lymphoma (ATLL) in a subject in need thereof, comprising administering to the subject an oligonucleotide inhibitor of miR-155 comprising a sequence selected from Table 2, wherein the oligonucleotide inhibitor of miR-155 decreases or maintains the tumor cell count in the subject.
110. A method for treating a disease associated with a Human T-cell Lymphotropic Virus (HTLV) in a subject in need thereof, comprising administering to the subject an oligonucleotide inhibitor of miR-155 comprising a sequence selected from Table 2.
111. The method of embodiment 106 or 110, wherein the HTLV is HTLV-1, HTLV-2, HTLV-3, and/or HTLV-4.
112. The method of embodiment 106, 110, or 111, wherein the disease associated with the HTLV is Adult T-cell Leukemia/Lymphoma (ATLL), HTLV-1 associated myelopathy/tropical spastic paraparesis (HAM/TSP), or HTLV-associated dermatitis.
113. The method of any one of embodiments 106, 110, 111, or 112, wherein the disease associated with the HTLV is ATLL.
114. The method of any one of embodiments 103-105, 107-109, and 112-113, wherein the ATLL is Acute ATLL, Lymphomatous ATLL, Chronic ATLL, or Smouldering ATLL.
115. The method of any one of embodiments 103-105, 107-109, and 112-114, wherein the ATLL is Leukemic ATLL.
116. The method of embodiment 115, wherein the Leukemic ATLL is Acute Leukemic ATLL.
117. The method of any one of embodiments 103-105, 107-109, and 112-114, wherein the ATLL is Lymphomatous ATLL.
118. The method of embodiment 117, wherein the Lymphomatous ATLL is Acute Lymphomatous ATLL.
119. The method of any one of embodiments 105, 109, 112, and 113, comprising administering to the subject one or more therapeutic agents for treating ATLL prior to or after the administration of the oligonucleotide inhibitor of miR-155.
120. The method of any one of embodiments 103-104, 107-108, and 119, wherein the one or more therapeutic agents are selected from the group consisting of zidovudine, interferon alpha, lenalidomide, etoposide, prednisone, vincristine, cyclophosphamide, doxorubicin hydrochloride, rituximab, romidepsin, mogamulizumab, pralatrexate, triamcinolone acetonide, ranimustine, vindesine, carboplatin, ALRN-6924 (a stapled alpha-helical peptide), methotrexate, gemcitabine, oxaliplatin, belinostat, acyclovir, Histone deacetylase (HDAC) inhibitors (HDIs), radiation therapy, and combinations thereof
121. The method of any one of embodiments 103-104, 107-108, and 119-120, wherein the one or more therapeutic agents are zidovudine, interferon alpha, lenalidomide, etoposide, prednisone, vincristine, cyclophosphamide, and doxorubicin hydrochloride.
122. The method of any one of embodiments 103-104, 107-108, and 119-120, wherein the one or more therapeutic agents are cyclophosphamide, doxorubicin, vincristine, etoposide, and prednisone.
123. The method of any one of embodiments 103-104, 107-108, and 119-120, wherein the one or more therapeutic agents are etoposide, zidovudine, interferon alpha, romidepsin, mogamulizumab, pralatrexate, lenalidomide, triamcinolone acetonide, and ALRN-6924.
124. The method of any one of embodiments 103-123, wherein the subject is receiving or has received a photochemotherapy.
125. The method of embodiment 124, wherein the photochemotherapy is a combination treatment comprising psoralens and long wave ultraviolet radiation.
126. The method of any one of embodiments 103-104, 107-108, and 119-123, wherein the one or more therapeutic agents for treating ATLL are administered to the subject at least for one cycle prior to the administration of the oligonucleotide inhibitor of miR-155.
127. The method of any one of embodiments 103-126, wherein the oligonucleotide inhibitor of miR-155 is administered according to a cycle, said cycle comprising:
   a. first administering at least one loading dose; and
   b. second administering at least one maintenance dose.
128. The method of embodiment 127, wherein the loading dose is about 2 times, 3 times, 4 times, or 5 times the maintenance dose.
129. The method of embodiment 127 or 128, wherein the loading dose is about 1500 mg to about 2400 mg.
130. The method of any one of embodiments 127-129, wherein the loading dose is about 1500 mg, 1800 mg, 2100 mg, or about 2400 mg.
131. The method of any one of embodiments 127-130, wherein the loading dose is about 1800 mg.
132. The method of any one of embodiments 127-131, wherein the loading dose is split into three doses administered every other day.
133. The method of any one of embodiments 127-132, wherein the loading dose is about 1800 mg split into three doses with each split dose being about 600 mg administered every other day.
134. The method of any one of embodiments 127-133, wherein the loading dose is about 1800 mg split into three doses of about 600 mg each administered on day 1, 3, and 5 of the cycle.
135. The method of any one of embodiments 127-134, wherein the maintenance dose is about 400 mg to about 1200 mg.
136. The method of any one of embodiments 127-135, wherein the maintenance dose is about 400 mg, 600 mg, 800 mg, 1000 mg, or about 1200 mg.
137. The method of any one of embodiments 127-136, wherein the maintenance dose is about 600 mg.
138. The method of any one of embodiments 127-137, wherein the maintenance dose is administered once a week.
139. The method of any one of embodiments 127-138, wherein the maintenance dose is about 600 mg administered once a week.
140. The method of any one of embodiments 103-139, wherein the oligonucleotide inhibitor of miR-155 comprises a sequence selected from Table 2.
141. The method of any one of embodiments 103-140, wherein the oligonucleotide inhibitor of miR-155 comprises the sequence of the sequence of 5′-lCs.dAs.lCs.dGs.dAs.lTs.lTs.dAs.lGs.dCs.lAs.lTs.lTs.lA-3′ (SEQ ID NO: 25).
142. The method of any one of embodiments 103-105, 107-109, and 113-141, wherein the administration of the oligonucleotide inhibitor of miR-155 decreases the tumor cell count compared to the tumor cell count at the beginning of the administration of the oligonucleotide inhibitor of miR-155.
143. The method of any one of embodiments 103-105, 107-109, and 113-142, wherein the administration of the oligonucleotide inhibitor of miR-155 decreases the tumor cell count compared to the tumor cell count at the beginning of the administration of the oligonucleotide inhibitor of miR-155 and maintains the tumor cell count at or below the decreased level.
144. The method of any one of embodiments 103-105, 107-109, and 113-143, wherein the administration of the oligonucleotide inhibitor of miR-155 decreases the tumor cell count by about 10 to 80% compared to the tumor cell count at the beginning of the administration of the oligonucleotide inhibitor of miR-155.
145. The method of any one of embodiments 103-105, 107-109, and 113-141, wherein the administration of the oligonucleotide inhibitor of miR-155 maintains the tumor cell count at or below the level that was present at the beginning of the administration of the oligonucleotide inhibitor of miR-155.
146. The method of any one of embodiments 103-105, 107-109, and 113-145, wherein the administration of the oligonucleotide inhibitor of miR-155 decreases the size of enlarged lymph nodes of the subject.
147. The method of any one of embodiments 103-105, 107-109, and 113-146, wherein the administration of the oligonucleotide inhibitor of miR-155 decreases the size of enlarged lymph nodes of the subject to a normal size.
148. The method of any one of embodiments 103-105, 107-109, and 113-147, wherein the administration of the one or more chemotherapeutic agents decreases the size of lymph nodes in the subject and the administration of the oligonucleotide inhibitor of miR-155 maintains the size of lymph nodes to the decreased size.
149. The method of any one of embodiments 103-105, 107-109, and 113-148, wherein the administration of the oligonucleotide inhibitor of miR-155 decreases the activation and/or proliferation of tumor cells.
150. The method of any one of embodiments 103-105, 107-109, and 113-149, wherein the administration of the oligonucleotide inhibitor of miR-155 decreases the activation of tumor cells as measured by the levels of activation markers HLA-DR and/or CD69.
151. The method of any one of embodiments 103-105, 107-109, and 113-150, wherein the administration of the oligonucleotide inhibitor of miR-155 decreases the levels of activation markers HLA-DR and/or CD69 on tumor cells by about 5-80% compared to the levels at the beginning of the administration of the oligonucleotide inhibitor of miR-155.
152. The method of any one of embodiments 103-105, 107-109, and 113-151, wherein the administration of the oligonucleotide inhibitor of miR-155 decreases the proliferation of tumor cells as measured by the levels of cell cycle marker Ki67.
153. The method of any one of embodiments 103-105, 107-109, and 113-152, wherein the administration of the oligonucleotide inhibitor of miR-155 decreases the levels of cell cycle marker Ki67 on tumor cells by about 5-80% compared to the levels at the beginning of the administration of the oligonucleotide inhibitor of miR-155.
154. The method of any one of embodiments 103-105, 107-109, and 113-152, wherein the administration of the oligonucleotide inhibitor of miR-155 decreases the proliferation of tumor cells by about 5-90% compared to the proliferation of tumor cells at the beginning of the administration of the oligonucleotide inhibitor of miR-155.
155. The method of any one of embodiments 103-154, wherein the oligonucleotide inhibitor of miR-155 is administered for about 6 week, 2 months, 3 months, 4 months, 5 months, 6 months, 8 months, 9 months, 10 months, or 12 months.
156. The method of any one of embodiments 103-105, 107-109, and 113-155, wherein the administration of the oligonucleotide inhibitor of miR-155 decreases the tumor cell count compared to the tumor cell count at the beginning of the administration of the oligonucleotide inhibitor of miR-155 and maintains the tumor cell count to the decreased level for 6 months or more.
157. The method of any one of embodiments 103-105, 107-109, and 113-155, wherein the administration of the oligonucleotide inhibitor of miR-155 maintains the tumor cell count to the level that was present at the beginning of the administration of the oligonucleotide inhibitor of miR-155 for 6 months or more.
158. The method of any one of embodiments 103-157, wherein the administration of the oligonucleotide inhibitor of miR-155 increases the progression-free survival of the subject by at least about 3 months, 4 months, 6 months, 8 months, 10 months, 12 months, 15 months, 18 months, 24 months or more.
159. The method of any one of embodiments 110-158, wherein the administration of the oligonucleotide inhibitor of miR-155 reduces the HTLV load in the subject.
160. The method of any one of embodiments 110-159, wherein the administration of the oligonucleotide inhibitor of miR-155 reduces the HTLV load in the subject by about 10-80%.
161. The method of any one of embodiments 103-160, wherein the administration of the oligonucleotide inhibitor of miR-155 promotes restoration of normal homeostatic immune response in the subject.
162. The method of any one of embodiments 103-161, wherein the administration of the oligonucleotide inhibitor of miR-155 promotes re-population of healthy B and/or T cells in the subject.
163. The method of any one of embodiments 103-162, wherein the administration of the oligonucleotide inhibitor of miR-155 increases the number of healthy B and/or T cells in the subject by about 5 to 60%.
164. A method for treating Adult T-cell Leukemia/Lymphoma (ATLL) in a subject in need thereof, comprising:
   a. administering to the subject one or more therapeutic agents for treating ATLL;
   b. administering an oligonucleotide inhibitor of miR-155 comprising the sequence of 5′-lCs.dAs.lCs.dGs.dAs.lTs.lTs.dAs.lGs.dCs.lAs.lTs.lTs.lA-3′ (SEQ ID NO: 25) to the subject after the administration of the one or more therapeutic agents, wherein the oligonucleotide inhibitor of miR-155 is administered according to a cycle, said cycle comprising:
   a. first administering at least one loading dose; and
   b. second administering at least one maintenance dose.
165. A method for decreasing or maintaining the tumor cell count in a subject suffering from Adult T-cell Leukemia/Lymphoma (ATLL), comprising administering an oligonucleotide inhibitor of miR-155 comprising the sequence of 5′-lCs.dAs.lCs.dGs.dAs.lTs.lTs.dAs.lGs.dCs.lAs.lTs.lTs.lA-3′ (SEQ ID NO: 25) to the subject, wherein the subject has received one or more therapeutic agents for treating ATLL prior to the treatment with the oligonucleotide inhibitor of miR-155, and wherein the oligonucleotide inhibitor of miR-155 is administered according to a cycle, said cycle comprising:
   a. first administering at least one loading dose; and
   b. second administering at least one maintenance dose.
166. A method for treating Adult T-cell Leukemia/Lymphoma (ATLL) in a subject in need thereof, comprising administering to the subject an oligonucleotide inhibitor of miR-155 comprising the sequence of 5′-lCs.dAs.lCs.dGs.dAs.lTs.lTs.dAs.lGs.dCs.lAs.lTs.lTs.lA-3′ (SEQ ID NO: 25), wherein the oligonucleotide inhibitor of miR-155 decreases or maintains the tumor cell count in the subject, and wherein the oligonucleotide inhibitor of miR-155 is administered according to a cycle, said cycle comprising:
   a. first administering at least one loading dose; and
   b. second administering at least one maintenance dose.
167. A method for treating Adult T-cell Leukemia/Lymphoma (ATLL) in a subject in need thereof, comprising:
   a. administering to the subject one or more therapeutic agents for treating ATLL;
   b. administering an oligonucleotide inhibitor of miR-155 comprising a sequence selected from Table 2 to the subject after the administration of the one or more therapeutic agents, wherein the oligonucleotide inhibitor of miR-155 is administered according to a cycle, said cycle comprising:
   a. first administering at least one loading dose; and
   b. second administering at least one maintenance dose.
168. A method for decreasing or maintaining the tumor cell count in a subject suffering from Adult T-cell Leukemia/Lymphoma (ATLL), comprising administering an oligonucleotide inhibitor of miR-155 comprising a sequence selected from Table 2 to the subject, wherein the subject has received one or more therapeutic agents for treating ATLL prior to the treatment with the oligonucleotide inhibitor of miR-155, and wherein the oligonucleotide inhibitor of miR-155 is administered according to a cycle, said cycle comprising:
   a. first administering at least one loading dose; and
   b. second administering at least one maintenance dose.
169. A method for treating Adult T-cell Leukemia/Lymphoma (ATLL) in a subject in need thereof, comprising administering to the subject an oligonucleotide inhibitor of miR-155 comprising a sequence selected from Table 2, wherein the oligonucleotide inhibitor of miR-155 decreases or maintains the tumor cell count in the subject, and wherein the oligonucleotide inhibitor of miR-155 is administered according to a cycle, said cycle comprising:
   a. first administering at least one loading dose; and
   b. second administering at least one maintenance dose.
170. The method of any one of embodiments 164-169, wherein the ATLL is Acute ATLL, Lymphomatous ATLL, Chronic ATLL, or Smouldering ATLL.
171. The method of any one of embodiments 164-170, wherein the ATLL is Leukemic ATLL.
172. The method of embodiment 171, wherein the Leukemic ATLL is Acute Leukemic ATLL.
173. The method of any one of embodiments 164-170, wherein the ATLL is Lymphomatous ATLL.
174. The method of embodiment 171, wherein the Lymphomatous ATLL is Acute Lymphomatous ATLL.
175. The method of embodiment 166 or 169, comprising administering to the subject one or more therapeutic agents for treating ATLL prior to or after the administration of the oligonucleotide inhibitor of miR-155.
176. The method of any one of embodiments 164, 165, 167, 168, and 170-175, wherein the one or more therapeutic agents are selected from the group consisting of zidovudine, interferon alpha, lenalidomide, etoposide, prednisone, vincristine, cyclophosphamide, doxorubicin hydrochloride, rituximab, romidepsin, mogamulizumab, pralatrexate, triamcinolone acetonide, ranimustine, vindesine, carboplatin, ALRN-6924 (a stapled alpha-helical peptide), methotrexate, gemcitabine, oxaliplatin, belinostat, acyclovir, Histone deacetylase (HDAC) inhibitors (HDIs), radiation therapy, and combinations thereof
177. The method of any one of embodiments 164, 165, 167, 168, and 170-175, wherein the one or more therapeutic agents are zidovudine, interferon alpha, lenalidomide, etoposide, prednisone, vincristine, cyclophosphamide, and doxorubicin hydrochloride.
178. The method of any one of embodiments 164, 165, 167, 168, and 170-175, wherein the one or more therapeutic agents are cyclophosphamide, doxorubicin, vincristine, etoposide, and prednisone.
179. The method of any one of embodiments 164, 165, 167, 168, and 170-175, wherein the one or more therapeutic agents are etoposide, zidovudine, interferon alpha, romidepsin, mogamulizumab, pralatrexate, lenalidomide, triamcinolone acetonide, and ALRN-6924.
180. The method of any one of embodiments 164-179, wherein the subject is receiving or has received a photochemotherapy.
181. The method of embodiment 180, wherein the photochemotherapy is a combination treatment comprising psoralens and long wave ultraviolet radiation.
182. The method of any one of embodiments 164, 165, 167, 168, and 170-181, wherein the one or more therapeutic agents for treating ATLL are administered to the subject at least for one cycle prior to the administration of the oligonucleotide inhibitor of miR-155.
183. The method of any one of embodiments 164-182, wherein the loading dose is about 2 times, 3 times, 4 times, or 5 times the maintenance dose.
184. The method of any one of embodiments 164-183, wherein the loading dose is about 1500 mg to about 2400 mg.
185. The method of any one of embodiments 164-184, wherein the loading dose is about 1500 mg, 1800 mg, 2100 mg, or about 2400 mg.
186. The method of any one of embodiments 164-185, wherein the loading dose is about 1800 mg.
187. The method of any one of embodiments 164-186, wherein the loading dose is split into three doses administered every other day.
188. The method of any one of embodiments 164-187, wherein the loading dose is about 1800 mg split into three doses with each split dose being about 600 mg administered every other day.
189. The method of any one of embodiments 164-188, wherein the loading dose is about 1800 mg split into three doses of about 600 mg each administered on day 1, 3, and 5 of the cycle.
190. The method of any one of embodiments 164-189, wherein the maintenance dose is about 400 mg to about 1200 mg.
191. The method of any one of embodiments 164-190, wherein the maintenance dose is about 400 mg, 600 mg, 800 mg, 1000 mg, or about 1200 mg.
192. The method of any one of embodiments 164-191, wherein the maintenance dose is about 600 mg.
193. The method of any one of embodiments 164-192, wherein the maintenance dose is administered once a week.
194. The method of any one of embodiments 164-193, wherein the maintenance dose is about 600 mg administered once a week.
195. The method of any one of embodiments 164-194, wherein the administration of the oligonucleotide inhibitor of miR-155 decreases the tumor cell count compared to the tumor cell count at the beginning of the administration of the oligonucleotide inhibitor of miR-155.
196. The method of any one of embodiments 164-195, wherein the administration of the oligonucleotide inhibitor of miR-155 decreases the tumor cell count compared to the tumor cell count at the beginning of the administration of the oligonucleotide inhibitor of miR-155 and maintains the tumor cell count at or below the decreased level.
197. The method of any one of embodiments 164-196, wherein the administration of the oligonucleotide inhibitor of miR-155 decreases the tumor cell count by about 10 to 80% compared to the tumor cell count at the beginning of the administration of the oligonucleotide inhibitor of miR-155.
198. The method of any one of embodiments 164-196, wherein the administration of the oligonucleotide inhibitor of miR-155 maintains the tumor cell count at or below the level that was present at the beginning of the administration of the oligonucleotide inhibitor of miR-155.
199. The method of any one of embodiments 164-198, wherein the administration of the oligonucleotide inhibitor of miR-155 decreases the size of enlarged lymph nodes of the subject.
200. The method of any one of embodiments 164-199, wherein the administration of the oligonucleotide inhibitor of miR-155 decreases the size of enlarged lymph nodes of the subject to a normal size.
201. The method of any one of embodiments 164-200, wherein the administration of the one or more chemotherapeutic agents decreases the size of lymph nodes in the subject and the administration of the oligonucleotide inhibitor of miR-155 maintains the size of lymph nodes to the decreased size.
202. The method of any one of embodiments 164-201, wherein the administration of the oligonucleotide inhibitor of miR-155 decreases the activation and/or proliferation of tumor cells.
203. The method of any one of embodiments 164-202, wherein the administration of the oligonucleotide inhibitor of miR-155 decreases the activation of tumor cells as measured by the levels of activation markers HLA-DR and/or CD69.
204. The method of any one of embodiments 164-203, wherein the administration of the oligonucleotide inhibitor of miR-155 decreases the levels of activation markers HLA-DR and/or CD69 on tumor cells by about 5-80% compared to the levels at the beginning of the administration of the oligonucleotide inhibitor of miR-155.
205. The method of any one of embodiments 164-204, wherein the administration of the oligonucleotide inhibitor of miR-155 decreases the proliferation of tumor cells as measured by the levels of cell cycle marker Ki67.
206. The method of any one of embodiments 164-205, wherein the administration of the oligonucleotide inhibitor of miR-155 decreases the levels of cell cycle marker Ki67 on tumor cells by about 5-80% compared to the levels at the beginning of the administration of the oligonucleotide inhibitor of miR-155.
207. The method of any one of embodiments 164-206, wherein the administration of the oligonucleotide inhibitor of miR-155 decreases the proliferation of tumor cells by about 5-90% compared to the proliferation of tumor cells at the beginning of the administration of the oligonucleotide inhibitor of miR-155.
208. The method of any one of embodiments 164-207, wherein the oligonucleotide inhibitor of miR-155 is administered for about 6 week, 2 months, 3 months, 4 months, 5 months, 6 months, 8 months, 9 months, 10 months, or 12 months.
209. The method of any one of embodiments 164-208, wherein the administration of the oligonucleotide inhibitor of miR-155 decreases the tumor cell count compared to the tumor cell count at the beginning of the administration of the oligonucleotide inhibitor of miR-155 and maintains the tumor cell count to the decreased level for 6 months or more.
210. The method of any one of embodiments 164-208, wherein the administration of the oligonucleotide inhibitor of miR-155 maintains the tumor cell count to the level that was present at the beginning of the administration of the oligonucleotide inhibitor of miR-155 for 6 months or more.
211. The method of any one of embodiments 164-210, wherein the administration of the oligonucleotide inhibitor of miR-155 increases the progression-free survival of the subject by at least about 3 months, 4 months, 6 months, 8 months, 10 months, 12 months, 15 months, 18 months, 24 months or more.
212. The method of any one of embodiments 164-211, wherein the administration of the oligonucleotide inhibitor of miR-155 reduces the HTLV load in the subject.
213. The method of any one of embodiments 164-212, wherein the administration of the oligonucleotide inhibitor of miR-155 reduces the HTLV load in the subject by about 10-80%.
214. The method of any one of embodiments 164-213, wherein the administration of the oligonucleotide inhibitor of miR-155 promotes restoration of normal homeostatic immune response in the subject.
215. The method of any one of embodiments 164-214, wherein the administration of the oligonucleotide inhibitor of miR-155 promotes re-population of healthy B and/or T cells in the subject.
216. The method of any one of embodiments 164-215, wherein the administration of the oligonucleotide inhibitor of miR-155 increases the number of healthy B and/or T cells in the subject by about 5 to 60%.
217. The method of any one of embodiments 1-216, wherein after a single intravenous administration of a 600 mg dose of the oligonucleotide inhibitor of miR-155 to a human, the Cmax of the oligonucleotide inhibitor ranges from 80%-125% of about 23 μg/mL.
218. The method of any one of embodiments 1-216, wherein after multiple intravenous administrations of a 600 mg dose of the oligonucleotide inhibitor of miR-155 to a human, the Cmax of the oligonucleotide inhibitor ranges from 80%-125% of about 17 μg/mL.
219. The method of any one of embodiments 1-218, wherein after a single intravenous administration of a 600 mg dose of the oligonucleotide inhibitor of miR-155 to a human, the AUC₀₋₂₄ of the oligonucleotide inhibitor ranges from 80%-125% of about 79 μg*hr/mL.
220. The method of any one of embodiments 1-218, wherein after multiple intravenous administrations of a 600 mg dose of the oligonucleotide inhibitor of miR-155 to a human, the AUC₀₋₂₄ of the oligonucleotide inhibitor ranges from 80%-125% of about 76 μg*hr/mL.
221. The method of any one of embodiments 1-220, wherein after a single intravenous administration of a 600 mg dose of the oligonucleotide inhibitor of miR-155 to a human, the AUC_{inf} of the oligonucleotide inhibitor ranges from 80%-125% of about 82 μg*hr/mL.
222. The method of any one of embodiments 1-220, wherein after multiple intravenous administrations of a 600 mg dose of the oligonucleotide inhibitor of miR-155 to a human, the AUC_{inf} of the oligonucleotide inhibitor ranges from 80%-125% of about 81 μg*hr/mL.
223. The method of any one of embodiments 1-222, wherein after a single intravenous administration of a 600 mg dose of the oligonucleotide inhibitor of miR-155 to a human, the tₘₐₓ of the oligonucleotide inhibitor is about 1.9 hours.
224. The method of any one of embodiments 1-222, wherein after multiple intravenous administrations of a 600 mg dose of the oligonucleotide inhibitor of miR-155 to a human, the tₘₐₓ of the oligonucleotide inhibitor is about 1.9 hours.

1. A method for treating Adult T-cell Leukemia/Lymphoma (ATLL) in a subject in need thereof, comprising:
a. administering to the subject one or more therapeutic agents for treating ATLL;
b. administering an oligonucleotide inhibitor of miR-155 to the subject after the administration of the one or more therapeutic agents.

2-6. (canceled)

7. The method of claim 1, wherein the ATLL is Acute ATLL, Chronic ATLL, Smouldering ATLL, or Lymphomatous ATLL;
wherein, optionally, the ATLL is Acute ATLL; wherein, optionally, the ATLL is ef Lymphomatous ATLL.

8-9. (canceled)

10. The method of claim 1, wherein the one or more therapeutic agents are selected from the group consisting of zidovudine, interferon alpha, lenalidomide, etoposide, prednisone, vincristine, cyclophosphamide, doxorubicin hydrochloride, rituximab, romidepsin, mogamulizumab, pralatrexate, triamcinolone acetonide, ranimustine, vindesine, carboplatin, ALRN-6924 (a stapled alpha-helical peptide), methotrexate, gemcitabine, oxaliplatin, belinostat, acyclovir, Histone deacetylase (HDAC) inhibitors (HDIs), radiation therapy, and combinations thereof.

11-16. (canceled)

17. The method of claim 1, wherein the oligonucleotide inhibitor of miR-155 is administered according to a cycle, said cycle comprising:
a. first administering at least one loading dose; and
b. second administering at least one maintenance dose;
wherein the loading dose is about 2 times, 3 times, 4 times, or 5 times the maintenance dose;
wherein the loading dose is about 1500 mg to about 2400 mg, wherein, optionally, the loading dose is about 1500 mg, 1800 mg, 2100 mg, or about 2400 mg.

18-21. (canceled)

22. The method of claim 17, wherein the loading dose is split into three doses administered every other day; wherein, optionally, the loading dose is about 1800 mg split into three doses with each split dose being about 600 mg administered every other day.

23-24. (canceled)

25. The method of claim 17, wherein the maintenance dose is about 400 mg to about 1200 mg; wherein, optionally, the maintenance dose is about 400 mg, 600 mg, 800 mg, 1000 mg, or about 1200 mg; wherein, optionally, the maintenance dose is administered once a week.

26-29. (canceled)

30. The method of claim 1, wherein the oligonucleotide inhibitor of miR-155 comprises the sequence selected from Table 2.

31. The method of claim 30, wherein the oligonucleotide inhibitor of miR-155 comprises the sequence of the sequence of 5′-lCs.dAs.lCs.dGs.dAs.lTs.lTs.dAs.lGs.dCs.lAs.lTs.lTs.lA-3′.

32. The method of claim 1, wherein the administration of the oligonucleotide inhibitor of miR-155 decreases the tumor cell count compared to the tumor cell count at the beginning of the administration of the oligonucleotide inhibitor of miR-155; wherein, optionally, the tumor cell count is decreased by about 10 to 80% compared to the tumor cell count at the beginning of the administration of the oligonucleotide inhibitor of miR-155; wherein, optionally, the administration of the oligonucleotide inhibitor of miR-155 maintains the tumor cell count at or below the decreased level; wherein, optionally, the administration of the oligonucleotide inhibitor of miR-155 maintains the tumor cell count at about the decreased level for 6 months or more.

33-35. (canceled)

36. The method of claim 1, wherein the administration of the oligonucleotide inhibitor of miR-155 decreases the size of enlarged lymph nodes of the subject; wherein, optionally, the lymph nodes are decreased to a normal size.

37. (canceled)

38. The method of claim 1, wherein the administration of the one or more chemotherapeutic agents decreases the size of lymph nodes in the subject and the administration of the oligonucleotide inhibitor of miR-155 maintains the size of lymph nodes at about the decreased size.

39. The method of claim 1, wherein the administration of the oligonucleotide inhibitor of miR-155 decreases the activation and/or proliferation of tumor cells.

40. The method of claim 39, wherein the administration of the oligonucleotide inhibitor of miR-155 decreases the activation of tumor cells as measured by the levels of activation markers HLA-DR and/or CD69; wherein, optionally, the levels of activation markers HLA-DR and/or CD69 on tumor cells is decreased by about 5-80% compared to the levels at the beginning of the administration of the oligonucleotide inhibitor of miR-155.

41. (canceled)

42. The method of claim 39, wherein the administration of the oligonucleotide inhibitor of miR-155 decreases the proliferation of tumor cells as measured by the levels of cell cycle marker Ki67; wherein, optionally, the levels of cell cycle marker Ki67 on tumor cells is decreased by about 5-80% compared to the levels at the beginning of the administration of the oligonucleotide inhibitor of miR-155.

43. (canceled)

44. The method of claim 1, wherein the administration of the oligonucleotide inhibitor of miR-155 decreases the proliferation of tumor cells by about 5-90% compared to the proliferation of tumor cells at the beginning of the administration of the oligonucleotide inhibitor of miR-155.

45. The method of claim 1, wherein the oligonucleotide inhibitor of miR-155 is administered for about 6 weeks, 2 months, 3 months, 4 months, 5 months, 6 months, 8 months, 9 months, 10 months, 12 months, or more.

46-47. (canceled)

48. The method of claim 1, wherein the administration of the oligonucleotide inhibitor of miR-155 increases the progression-free survival of the subject by at least about 3 months, 4 months, 6 months, 8 months, 10 months, 12 months, 15 months, 18 months, 24 months or more.

49. The method of claim 17, wherein the administration of the oligonucleotide inhibitor of miR-155 reduces the HTLV load in the subject; wherein, optionally, the administration of the oligonucleotide inhibitor of miR-155 reduces the HTLV load in the subject by about 10-80%.

50. (canceled)

51. The method of claim 1, wherein the administration of the oligonucleotide inhibitor of miR-155 promotes restoration of normal homeostatic immune cell populations in the subject.

52-103. (canceled)

104. The method of claim 1, wherein the administration of the oligonucleotide inhibitor of miR-155 increases apoptosis of tumor cells as measured by the levels of apoptosis marker cPARP; wherein, optionally, the apoptosis of tumor cells is increased by about 5-90% compared to the apoptosis of tumor cells at the beginning of the administration of the oligonucleotide inhibitor of miR-155.

105-117. (canceled)


SEQUENCE LISTING (120 sequences)

SEQ ID NO: 1 - RNA, 23 nt, Homo sapiens
uuaaugcuaaucgugauaggggu

SEQ ID NO: 2 - RNA, 22 nt, Homo sapiens
cuccuacauauuagcauuaaca

SEQ ID NO: 3 - DNA, 16 bp, Artificial Sequence
  miR-155 10101
  misc_feature (1)..(16): joined through phosphorothioate bonds
  modified_base (1)..(1): locked nucleic acid adenosine
  modified_base (5)..(5): locked nucleic acid cytidine
  modified_base (6)..(6): locked nucleic acid guanosine
  modified_base (8)..(8): locked nucleic acid thymidine
  modified_base (10)..(10): locked nucleic acid adenosine
  modified_base (11)..(11): locked nucleic acid guanosine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 4 - DNA, 16 bp, Artificial Sequence
  miR-155 11293
  misc_feature (1)..(16): joined through phosphorothioate bonds
  modified_base (1)..(1): locked nucleic acid adenosine
  modified_base (5)..(5): locked nucleic acid cytidine
  modified_base (6)..(6): locked nucleic acid guanosine
  modified_base (9)..(9): locked nucleic acid thymidine
  modified_base (10)..(10): locked nucleic acid adenosine
  modified_base (11)..(11): locked nucleic acid guanosine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 5 - DNA, 16 bp, Artificial Sequence
  miR-155 11294
  misc_feature (1)..(16): joined through phosphorothioate bonds
  modified_base (1)..(1): locked nucleic acid adenosine
  modified_base (2)..(2): locked nucleic acid thymidine
  modified_base (6)..(6): locked nucleic acid guanosine
  modified_base (8)..(8): locked nucleic acid thymidine
  modified_base (10)..(10): locked nucleic acid adenosine
  modified_base (11)..(11): locked nucleic acid guanosine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 6 - DNA, 16 bp, Artificial Sequence
  miR-155 11295
  misc_feature (1)..(16): joined through phosphorothioate bonds
  modified_base (1)..(1): locked nucleic acid adenosine
  modified_base (2)..(2): locked nucleic acid thymidine
  modified_base (6)..(6): locked nucleic acid guanosine
  modified_base (7)..(7): locked nucleic acid adenosine
  modified_base (10)..(10): locked nucleic acid adenosine
  modified_base (11)..(11): locked nucleic acid guanosine
  modified_base (12)..(12): locked nucleic acid cytidine
  modified_base (14)..(14): locked nucleic acid thymidine
  modified_base (16)..(16): locked nucleic acid adenosine
atcacgattagcatta

SEQ ID NO: 7 - DNA, 16 bp, Artificial Sequence
  miR-155 11296
  misc_feature (1)..(16): joined through phosphorothioate bonds
  modified_base (1)..(1): locked nucleic acid adenosine
  modified_base (5)..(5): locked nucleic acid cytidine
  modified_base (6)..(6): locked nucleic acid guanosine
  modified_base (8)..(8): locked nucleic acid thymidine
  modified_base (10)..(10): locked nucleic acid adenosine
  modified_base (11)..(11): locked nucleic acid guanosine
  modified_base (13)..(13): locked nucleic acid adenosine
  modified_base (14)..(14): locked nucleic acid thymidine
  modified_base (16)..(16): locked nucleic acid adenosine
atcacgattagcatta

SEQ ID NO: 8 - DNA, 16 bp, Artificial Sequence
  miR-155 16 6
  misc_feature (1)..(16): joined through phosphorothioate bonds
  modified_base (1)..(1): locked nucleic acid adenosine
  modified_base (2)..(2): locked nucleic acid thymidine
  modified_base (5)..(5): locked nucleic acid cytidine
  modified_base (9)..(9): locked nucleic acid thymidine
  modified_base (10)..(10): locked nucleic acid adenosine
  modified_base (12)..(12): locked nucleic acid cytidine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 9 - DNA, 16 bp, Artificial Sequence
  miR-155 16 7
  misc_feature (1)..(16): joined through phosphorothioate bonds
  modified_base (1)..(1): locked nucleic acid adenosine
  modified_base (5)..(5): locked nucleic acid cytidine
  modified_base (7)..(7): locked nucleic acid adenosine
  modified_base (9)..(9): locked nucleic acid thymidine
  modified_base (10)..(10): locked nucleic acid adenosine
  modified_base (12)..(12): locked nucleic acid cytidine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 10 - DNA, 16 bp, Artificial Sequence
  miR-155 16 8
  misc_feature (1)..(16): joined through phosphorothioate bonds
  modified_base (1)..(1): locked nucleic acid adenosine
  modified_base (4)..(4): locked nucleic acid adenosine
  modified_base (7)..(7): locked nucleic acid adenosine
  modified_base (8)..(8): locked nucleic acid thymidine
  modified_base (10)..(10): locked nucleic acid adenosine
  modified_base (11)..(11): locked nucleic acid guanosine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 11 - DNA, 16 bp, Artificial Sequence
  miR-155 16 9
  misc_feature (1)..(16): joined through phosphorothioate bonds
  modified_base (1)..(1): locked nucleic acid adenosine
  modified_base (3)..(3): locked nucleic acid cytidine
  modified_base (6)..(6): locked nucleic acid guanosine
  modified_base (8)..(9): locked nucleic acid thymidine
  modified_base (12)..(12): locked nucleic acid cytidine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 12 - DNA, 16 bp, Artificial Sequence
  miR-155 16 10
  misc_feature (1)..(16): joined through phosphorothioate bonds
  modified_base (1)..(1): locked nucleic acid adenosine
  modified_base (2)..(2): locked nucleic acid thymidine
  modified_base (4)..(4): locked nucleic acid adenosine
  modified_base (5)..(5): locked nucleic acid cytidine
  modified_base (9)..(9): locked nucleic acid thymidine
  modified_base (10)..(10): locked nucleic acid adenosine
  modified_base (12)..(12): locked nucleic acid cytidine
  modified_base (13)..(13): locked nucleic acid adenosine
  modified_base (16)..(16): locked nucleic acid adenosine
atcacgattagcatta

SEQ ID NO: 13 - DNA, 16 bp, Artificial Sequence
  miR-155 16 11
  misc_feature (1)..(16): joined through phosphorothioate bonds
  modified_base (1)..(1): locked nucleic acid adenosine
  modified_base (3)..(3): locked nucleic acid cytidine
  modified_base (7)..(7): locked nucleic acid adenosine
  modified_base (9)..(9): locked nucleic acid thymidine
  modified_base (10)..(10): locked nucleic acid adenosine
  modified_base (12)..(12): locked nucleic acid cytidine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 14 - DNA, 16 bp, Artificial Sequence
  miR-155 16 12
  misc_feature (1)..(16): joined through phosphorothioate bonds
  modified_base (1)..(1): locked nucleic acid adenosine
  modified_base (3)..(3): locked nucleic acid cytidine
  modified_base (5)..(5): locked nucleic acid cytidine
  modified_base (7)..(7): locked nucleic acid adenosine
  modified_base (9)..(9): locked nucleic acid thymidine
  modified_base (11)..(11): locked nucleic acid guanosine
  modified_base (13)..(13): locked nucleic acid adenosine
  modified_base (15)..(15): locked nucleic acid thymidine
  modified_base (16)..(16): locked nucleic acid adenosine
atcacgattagcatta

SEQ ID NO: 15 - DNA, 15 bp, Artificial Sequence
  miR-155 15 1
  misc_feature (1)..(15): joined through phosphorothioate bonds
  modified_base (1)..(1): locked nucleic acid thymidine
  modified_base (4)..(4): locked nucleic acid cytidine
  modified_base (7)..(7): locked nucleic acid thymidine
  modified_base (10)..(10): locked nucleic acid guanosine
  modified_base (12)..(12): locked nucleic acid adenosine
  modified_base (13)..(13): locked nucleic acid thymidine
  modified_base (15)..(15): locked nucleic acid adenosine
tcacgattagcatta

SEQ ID NO: 16 - DNA, 15 bp, Artificial Sequence
  miR-155 15 2
  misc_feature (1)..(15): joined through phosphorothioate bonds
  modified_base (1)..(1): locked nucleic acid thymidine
  modified_base (3)..(3): locked nucleic acid adenosine
  modified_base (6)..(6): locked nucleic acid adenosine
  modified_base (7)..(7): locked nucleic acid thymidine
  modified_base (10)..(10): locked nucleic acid guanosine
  modified_base (12)..(12): locked nucleic acid adenosine
  modified_base (14)..(14): locked nucleic acid thymidine
  modified_base (15)..(15): locked nucleic acid adenosine
tcacgattagcatta

SEQ ID NO: 17 - DNA, 15 bp, Artificial Sequence
  miR-155 15 3
  misc_feature (1)..(15): joined through phosphorothioate bonds
  modified_base (1)..(1): locked nucleic acid thymidine
  modified_base (5)..(5): locked nucleic acid guanosine
  modified_base (6)..(6): locked nucleic acid adenosine
  modified_base (7)..(7): locked nucleic acid thymidine
  modified_base (10)..(10): locked nucleic acid guanosine
  modified_base (12)..(12): locked nucleic acid adenosine
  modified_base (14)..(14): locked nucleic acid thymidine
  modified_base (15)..(15): locked nucleic acid adenosine
tcacgattagcatta

SEQ ID NO: 18 - DNA, 15 bp, Artificial Sequence
  miR-155 15 10823
  misc_feature (1)..(15): joined through phosphorothioate bonds
  modified_base (1)..(1): locked nucleic acid thymidine
  modified_base (2)..(2): locked nucleic acid cytidine
  modified_base (3)..(3): locked nucleic acid adenosine
  modified_base (5)..(5): locked nucleic acid guanosine
  modified_base (8)..(8): locked nucleic acid thymidine
  modified_base (9)..(9): locked nucleic acid adenosine
  modified_base (11)..(11): locked nucleic acid cytidine
  modified_base (14)..(14): locked nucleic acid thymidine
  modified_base (15)..(15): locked nucleic acid adenosine
tcacgattagcatta

SEQ ID NO: 19 - DNA, 15 bp, Artificial Sequence
  miR-155 15 10824
  misc_feature (1)..(15): joined through phosphorothioate bonds
  modified_base (1)..(1): locked nucleic acid thymidine
  modified_base (4)..(4): locked nucleic acid cytidine
  modified_base (8)..(8): locked nucleic acid thymidine
  modified_base (9)..(9): locked nucleic acid adenosine
  modified_base (10)..(10): locked nucleic acid guanosine
  modified_base (11)..(11): locked nucleic acid cytidine
  modified_base (12)..(12): locked nucleic acid adenosine
  modified_base (13)..(14): locked nucleic acid thymidine
  modified_base (15)..(15): locked nucleic acid adenosine
tcacgattagcatta

SEQ ID NO: 20 - DNA, 15 bp, Artificial Sequence
  miR-155 15 4
  misc_feature (1)..(15): joined through phosphorothioate bonds
  modified_base (1)..(1): locked nucleic acid thymidine
  modified_base (3)..(3): locked nucleic acid adenosine
  modified_base (5)..(5): locked nucleic acid guanosine
  modified_base (6)..(6): locked nucleic acid adenosine
  modified_base (7)..(7): locked nucleic acid thymidine
  modified_base (10)..(10): locked nucleic acid guanosine
  modified_base (11)..(11): locked nucleic acid cytidine
  modified_base (12)..(12): locked nucleic acid adenosine
  modified_base (14)..(14): locked nucleic acid thymidine
  modified_base (15)..(15): locked nucleic acid adenosine
tcacgattagcatta

SEQ ID NO: 21 - DNA, 11 bp, Artificial Sequence
  miR-155 11 1
  misc_feature (1)..(11): joined through phosphorothioate bonds
  modified_base (1)..(1): locked nucleic acid guanosine
  modified_base (2)..(2): locked nucleic acid adenosine
  modified_base (3)..(4): locked nucleic acid thymidine
  modified_base (5)..(5): locked nucleic acid adenosine
  modified_base (6)..(6): locked nucleic acid guanosine
  modified_base (8)..(8): locked nucleic acid adenosine
  modified_base (9)..(9): locked nucleic acid thymidine
  modified_base (11)..(11): locked nucleic acid adenosine
gattagcatta

SEQ ID NO: 22 - DNA, 12 bp, Artificial Sequence
  miR-155 12 1
  misc_feature (1)..(12): joined through phosphorothioate bonds
  modified_base (1)..(1): locked nucleic acid cytidine
  modified_base (3)..(3): locked nucleic acid adenosine
  modified_base (4)..(5): locked nucleic acid thymidine
  modified_base (6)..(6): locked nucleic acid adenosine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 23 - DNA, 12 bp, Artificial Sequence
  miR-155 12 2
  misc_feature (1)..(12): joined through phosphorothioate bonds
  modified_base (1)..(1): locked nucleic acid cytidine
  modified_base (3)..(3): locked nucleic acid adenosine
  modified_base (4)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 24 - DNA, 14 bp, Artificial Sequence
  miR-155 14 1
  misc_feature (1)..(14): joined through phosphorothioate bonds
  modified_base (1)..(1): locked nucleic acid cytidine
  modified_base (2)..(2): locked nucleic acid adenosine
  modified_base (4)..(4): locked nucleic acid guanosine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 25 - DNA, 14 bp, Artificial Sequence
  miR-155 14 2
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 26 - DNA, 15 bp, Artificial Sequence
  11622 Me C-DNA
  misc_feature (1)..(15): joined through phosphorothioate bonds
  modified_base (1)..(1): locked nucleic acid thymidine
  modified_base (3)..(3): locked nucleic acid adenosine
  modified_base (4)..(4): 5-Methylcytosine
  modified_base (5)..(5): locked nucleic acid guanosine
  modified_base (6)..(6): locked nucleic acid adenosine
  modified_base (7)..(7): locked nucleic acid thymidine
  modified_base (10)..(10): locked nucleic acid guanosine
  modified_base (11)..(11): locked nucleic acid cytidine
  modified_base (12)..(12): locked nucleic acid adenosine
  modified_base (14)..(14): locked nucleic acid thymidine
  modified_base (15)..(15): locked nucleic acid adenosine
tcacgattagcatta

SEQ ID NO: 27 - DNA, 8 bp, Artificial Sequence
  miR-155 8
  misc_feature (1)..(8): joined through phosphorothioate bonds
  modified_base (1)..(1): locked nucleic acid thymidine
  modified_base (2)..(2): locked nucleic acid adenosine
  modified_base (3)..(3): locked nucleic acid guanosine
  modified_base (4)..(4): locked nucleic acid cytidine
  modified_base (5)..(5): locked nucleic acid adenosine
  modified_base (6)..(7): locked nucleic acid thymidine
  modified_base (8)..(8): locked nucleic acid adenosine
tagcatta

SEQ ID NO: 28 - DNA, 14 bp, Artificial Sequence
  miR-155 mimic 11667
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 29 - DNA, 14 bp, Artificial Sequence
  miR-155 mimic 12707
  misc_feature (1)..(14): joined through phosphorothioate bonds
  modified_base (1)..(1): locked nucleic acid cytidine
  modified_base (3)..(3): locked nucleic acid cytidine
  modified_base (5)..(5): locked nucleic acid adenosine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 30 - DNA, 14 bp, Artificial Sequence
  miR-155 mimic 12708
  misc_feature (1)..(14): joined through phosphorothioate bonds
  modified_base (1)..(1): locked nucleic acid cytidine
  modified_base (3)..(3): locked nucleic acid cytidine
  modified_base (5)..(5): locked nucleic acid adenosine
  modified_base (7)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 31 - DNA, 14 bp, Artificial Sequence
  miR-155 mimic 12709
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (8)..(8): locked nucleic acid adenosine
  modified_base (9)..(9): locked nucleic acid guanosine
  modified_base (11)..(11): locked nucleic acid adenosine
  modified_base (13)..(13): locked nucleic acid thymidine
  modified_base (14)..(14): locked nucleic acid adenosine
cacgattagcatta

SEQ ID NO: 32 - DNA, 14 bp, Artificial Sequence
  miR-155 mimic 12868
  misc_feature (1)..(14): joined through phosphorothioate bonds
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 33 - DNA, 14 bp, Artificial Sequence
  miR-155 mimic 12869
  misc_feature (1)..(14): joined through phosphorothioate bonds
  modified_base (1)..(1): locked nucleic acid cytidine
  modified_base (2)..(2): locked nucleic acid adenosine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 34 - DNA, 14 bp, Artificial Sequence
  miR-155 mimic 12870
  misc_feature (1)..(14): joined through phosphorothioate bonds
  modified_base (1)..(1): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 35 - DNA, 14 bp, Artificial Sequence
  miR-155 mimic 12871
  misc_feature (1)..(14): joined through phosphorothioate bonds
  modified_base (1)..(1): locked nucleic acid cytidine
  modified_base (3)..(3): locked nucleic acid cytidine
  modified_base (4)..(4): locked nucleic acid guanosine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 36 - DNA, 14 bp, Artificial Sequence
  miR-155 mimic 12872
  misc_feature (1)..(14): joined through phosphorothioate bonds
  modified_base (1)..(1): locked nucleic acid cytidine
  modified_base (3)..(3): locked nucleic acid cytidine
  modified_base (5)..(5): locked nucleic acid adenosine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 37 - DNA, 14 bp, Artificial Sequence
  miR-155 mimic 12873
  misc_feature (1)..(14): joined through phosphorothioate bonds
  modified_base (1)..(1): locked nucleic acid cytidine
  modified_base (3)..(3): locked nucleic acid cytidine
  modified_base (7)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 38 - DNA, 14 bp, Artificial Sequence
  miR-155 mimic 12874
  misc_feature (1)..(14): joined through phosphorothioate bonds
  modified_base (1)..(1): locked nucleic acid cytidine
  modified_base (3)..(3): locked nucleic acid cytidine
  modified_base (6)..(6): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 39 - DNA, 14 bp, Artificial Sequence
  miR-155 mimic 12875
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (8)..(8): locked nucleic acid adenosine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 40 - DNA, 14 bp, Artificial Sequence
  miR-155 mimic 12876
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 41 - DNA, 14 bp, Artificial Sequence
  miR-155 mimic 12877
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (10)..(10): locked nucleic acid cytidine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 42 - DNA, 14 bp, Artificial Sequence
  miR-155 mimic 12878
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (12)..(13): locked nucleic acid thymidine
  modified_base (14)..(14): locked nucleic acid adenosine
cacgattagcatta

SEQ ID NO: 43 - DNA, 14 bp, Artificial Sequence
  miR-155 mimic 12879
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (13)..(13): locked nucleic acid thymidine
  modified_base (14)..(14): locked nucleic acid adenosine
cacgattagcatta

SEQ ID NO: 44 - DNA, 14 bp, Artificial Sequence
  miR-155 mimic 12880
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(12): locked nucleic acid thymidine
  modified_base (14)..(14): locked nucleic acid adenosine
cacgattagcatta

SEQ ID NO: 45 - DNA, 14 bp, Artificial Sequence
  miR-155 mimic 12881
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
cacgattagcatta

SEQ ID NO: 46 - DNA, 14 bp, Artificial Sequence
  miR-155 mimic 12882
  misc_feature (1)..(14): joined through phosphorothioate bonds
  modified_base (2)..(2): locked nucleic acid adenosine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 47 - DNA, 14 bp, Artificial Sequence
  miR-155 mimic 12883
  misc_feature (1)..(14): joined through phosphorothioate bonds
  modified_base (1)..(1): locked nucleic acid cytidine
  modified_base (2)..(2): locked nucleic acid adenosine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 48 - DNA, 14 bp, Artificial Sequence
  miR-155 mimic 12884
  misc_feature (1)..(14): joined through phosphorothioate bonds
  modified_base (1)..(1): locked nucleic acid cytidine
  modified_base (4)..(4): locked nucleic acid guanosine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 49 - DNA, 14 bp, Artificial Sequence
  miR-155 mimic 12885
  misc_feature (1)..(14): joined through phosphorothioate bonds
  modified_base (1)..(1): locked nucleic acid cytidine
  modified_base (3)..(3): locked nucleic acid cytidine
  modified_base (5)..(5): locked nucleic acid adenosine
  modified_base (7)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 50 - DNA, 14 bp, Artificial Sequence
  miR-155 mimic 12886
  misc_feature (1)..(14): joined through phosphorothioate bonds
  modified_base (1)..(1): locked nucleic acid cytidine
  modified_base (3)..(3): locked nucleic acid cytidine
  modified_base (6)..(6): locked nucleic acid thymidine
  modified_base (8)..(8): locked nucleic acid adenosine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 51 - DNA, 14 bp, Artificial Sequence
  miR-155 mimic 12887
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (8)..(8): locked nucleic acid adenosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 52 - DNA, 14 bp, Artificial Sequence
  miR-155 mimic 12888
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (10)..(10): locked nucleic acid cytidine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 53 - DNA, 14 bp, Artificial Sequence
  miR-155 mimic 12889
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (10)..(10): locked nucleic acid cytidine
  modified_base (12)..(13): locked nucleic acid thymidine
  modified_base (14)..(14): locked nucleic acid adenosine
cacgattagcatta

SEQ ID NO: 54 - DNA, 13 bp, Artificial Sequence
  miR-155 mimic 12890
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 55 - DNA, 12 bp, Artificial Sequence
  miR-155 mimic 12891
  misc_feature (1)..(12): joined through phosphorothioate bonds
  modified_base (1)..(1): locked nucleic acid cytidine
  modified_base (4)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 56 - DNA, 11 bp, Artificial Sequence
  miR-155 mimic 12892
  misc_feature (1)..(11): joined through phosphorothioate bonds
  modified_base (1)..(1): locked nucleic acid guanosine
  modified_base (3)..(4): locked nucleic acid thymidine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 57 - DNA, 13 bp, Artificial Sequence
  miR-155 mimic 12893
  misc_feature (1)..(13): joined through phosphorothioate bonds
  modified_base (2)..(2): locked nucleic acid cytidine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 58 - DNA, 13 bp, Artificial Sequence
  miR-155 mimic 12894
  misc_feature (1)..(13): joined through phosphorothioate bonds
  modified_base (1)..(1): locked nucleic acid adenosine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 59 - DNA, 13 bp, Artificial Sequence
  miR-155 mimic 12895
  misc_feature (1)..(13): joined through phosphorothioate bonds
  modified_base (1)..(1): locked nucleic acid adenosine
  modified_base (2)..(2): locked nucleic acid cytidine
  modified_base (3)..(3): locked nucleic acid guanosine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 60 - DNA, 13 bp, Artificial Sequence
  miR-155 mimic 12896
  misc_feature (1)..(13): joined through phosphorothioate bonds
  modified_base (1)..(1): locked nucleic acid adenosine
  modified_base (2)..(2): locked nucleic acid cytidine
  modified_base (4)..(4): locked nucleic acid adenosine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 61 - DNA, 13 bp, Artificial Sequence
  miR-155 mimic 12897
  misc_feature (1)..(13): joined through phosphorothioate bonds
  modified_base (1)..(1): locked nucleic acid adenosine
  modified_base (2)..(2): locked nucleic acid cytidine
  modified_base (6)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 62 - DNA, 13 bp, Artificial Sequence
  miR-155 mimic 12898
  misc_feature (1)..(13): joined through phosphorothioate bonds
  modified_base (1)..(1): locked nucleic acid adenosine
  modified_base (2)..(2): locked nucleic acid cytidine
  modified_base (5)..(5): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 63 - DNA, 13 bp, Artificial Sequence
  miR-155 mimic 12899
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (7)..(7): locked nucleic acid adenosine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 64 - DNA, 13 bp, Artificial Sequence
  miR-155 mimic 12900
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 65 - DNA, 13 bp, Artificial Sequence
  miR-155 mimic 12901
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (9)..(9): locked nucleic acid cytidine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 66 - DNA, 13 bp, Artificial Sequence
  miR-155 mimic 12902
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (11)..(12): locked nucleic acid thymidine
  modified_base (13)..(13): locked nucleic acid adenosine
acgattagcatta

SEQ ID NO: 67 - DNA, 13 bp, Artificial Sequence
  miR-155 mimic 12903
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (10)..(10): locked nucleic acid adenosine
  modified_base (12)..(13): locked nucleic acid thymidine
acgattagcatta

SEQ ID NO: 68 - DNA, 13 bp, Artificial Sequence
  miR-155 mimic 12904
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (10)..(10): locked nucleic acid adenosine
  modified_base (11)..(11): locked nucleic acid thymidine
  modified_base (13)..(13): locked nucleic acid adenosine
acgattagcatta

SEQ ID NO: 69 - DNA, 13 bp, Artificial Sequence
  miR-155 mimic 12905
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
acgattagcatta

SEQ ID NO: 70 - DNA, 13 bp, Artificial Sequence
  miR-155 mimic 12906
  misc_feature (1)..(13): joined through phosphorothioate bonds
  modified_base (1)..(1): locked nucleic acid adenosine
  modified_base (3)..(3): locked nucleic acid guanosine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 71 - DNA, 13 bp, Artificial Sequence
  miR-155 mimic 12907
  misc_feature (1)..(13): joined through phosphorothioate bonds
  modified_base (1)..(1): locked nucleic acid adenosine
  modified_base (2)..(2): locked nucleic acid cytidine
  modified_base (4)..(4): locked nucleic acid adenosine
  modified_base (6)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 72 - DNA, 13 bp, Artificial Sequence
  miR-155 mimic 12908
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 73 - DNA, 13 bp, Artificial Sequence
  miR-155 mimic 12909
  misc_feature (1)..(13): joined through phosphorothioate bonds
  modified_base (1)..(1): locked nucleic acid adenosine
  modified_base (2)..(2): locked nucleic acid cytidine
  modified_base (5)..(5): locked nucleic acid thymidine
  modified_base (7)..(7): locked nucleic acid adenosine
  modified_base (8)..(8): locked nucleic acid guanosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 74 - DNA, 13 bp, Artificial Sequence
  miR-155 mimic 12910
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (7)..(7): locked nucleic acid adenosine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 75 - DNA, 13 bp, Artificial Sequence
  miR-155 mimic 12911
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (9)..(9): locked nucleic acid cytidine
  modified_base (10)..(10): locked nucleic acid adenosine
  modified_base (11)..(12): locked nucleic acid thymidine
  modified_base (13)..(13): locked nucleic acid adenosine
acgattagcatta

SEQ ID NO: 76 - DNA, 13 bp, Artificial Sequence
  miR-155 mimic 12912
  misc_feature (1)..(13): joined through phosphorothioate bonds
  modified_base (1)..(1): locked nucleic acid adenosine
  modified_base (2)..(2): locked nucleic acid cytidine
  modified_base (5)..(6): locked nucleic acid thymidine
  modified_base (8)..(8): locked nucleic acid guanosine
  modified_base (9)..(9): locked nucleic acid cytidine
  modified_base (11)..(12): locked nucleic acid thymidine
  modified_base (13)..(13): locked nucleic acid adenosine
acgattagcatta

SEQ ID NO: 77 - DNA, 12 bp, Artificial Sequence
  miR-155 mimic 12913
  misc_feature (1)..(12): joined through phosphorothioate bonds
  modified_base (4)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 78 - DNA, 12 bp, Artificial Sequence
  miR-155 mimic 12914
  misc_feature (1)..(12): joined through phosphorothioate bonds
  modified_base (1)..(1): locked nucleic acid cytidine
  modified_base (2)..(2): locked nucleic acid guanosine
  modified_base (4)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 79 - DNA, 12 bp, Artificial Sequence
  miR-155 mimic 12915
  misc_feature (1)..(12): joined through phosphorothioate bonds
  modified_base (1)..(1): locked nucleic acid cytidine
  modified_base (3)..(3): locked nucleic acid adenosine
  modified_base (4)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 80 - DNA, 12 bp, Artificial Sequence
  miR-155 mimic 12916
  misc_feature (1)..(12): joined through phosphorothioate bonds
  modified_base (1)..(1): locked nucleic acid cytidine
  modified_base (5)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 81 - DNA, 12 bp, Artificial Sequence
  miR-155 mimic 12917
  misc_feature (1)..(12): joined through phosphorothioate bonds
  modified_base (1)..(1): locked nucleic acid cytidine
  modified_base (4)..(4): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 82 - DNA, 12 bp, Artificial Sequence
  miR-155 mimic 12918
  misc_feature (1)..(12): joined through phosphorothioate bonds
  modified_base (1)..(1): locked nucleic acid cytidine
  modified_base (4)..(5): locked nucleic acid thymidine
  modified_base (6)..(6): locked nucleic acid adenosine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 83 - DNA, 12 bp, Artificial Sequence
  miR-155 mimic 12919
  misc_feature (1)..(12): joined through phosphorothioate bonds
  modified_base (1)..(1): locked nucleic acid cytidine
  modified_base (4)..(5): locked nucleic acid thymidine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 84 - DNA, 12 bp, Artificial Sequence
  miR-155 mimic 12920
  misc_feature (1)..(12): joined through phosphorothioate bonds
  modified_base (1)..(1): locked nucleic acid cytidine
  modified_base (4)..(5): locked nucleic acid thymidine
  modified_base (7)..(7): locked nucleic acid guanosine
  modified_base (8)..(8): locked nucleic acid cytidine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 85 - DNA, 12 bp, Artificial Sequence
  miR-155 mimic 12921
  misc_feature (1)..(12): joined through phosphorothioate bonds
  modified_base (1)..(1): locked nucleic acid cytidine
  modified_base (4)..(5): locked nucleic acid thymidine
  modified_base (7)..(7): locked nucleic acid guanosine
  modified_base (10)..(11): locked nucleic acid thymidine
  modified_base (12)..(12): locked nucleic acid adenosine
cgattagcatta

SEQ ID NO: 86 - DNA, 12 bp, Artificial Sequence
  miR-155 mimic 12922
  misc_feature (1)..(16): joined through phosphorothioate bonds
  modified_base (1)..(1): locked nucleic acid cytidine
  modified_base (4)..(5): locked nucleic acid thymidine
  modified_base (7)..(7): locked nucleic acid guanosine
  modified_base (9)..(9): locked nucleic acid adenosine
  modified_base (11)..(12): locked nucleic acid thymidine
cgattagcatta

SEQ ID NO: 87 - DNA, 12 bp, Artificial Sequence
  miR-155 mimic 12923
  misc_feature (1)..(12): joined through phosphorothioate bonds
  modified_base (1)..(1): locked nucleic acid cytidine
  modified_base (4)..(5): locked nucleic acid thymidine
  modified_base (7)..(7): locked nucleic acid guanosine
  modified_base (9)..(9): locked nucleic acid adenosine
  modified_base (10)..(10): locked nucleic acid thymidine
  modified_base (12)..(12): locked nucleic acid adenosine
cgattagcatta

SEQ ID NO: 88 - DNA, 12 bp, Artificial Sequence
  miR-155 mimic 12924
  misc_feature (1)..(12): joined through phosphorothioate bonds
  modified_base (1)..(1): locked nucleic acid cytidine
  modified_base (4)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
cgattagcatta

SEQ ID NO: 89 - DNA, 12 bp, Artificial Sequence
  miR-155 mimic 12925
  misc_feature (1)..(12): joined through phosphorothioate bonds
  modified_base (2)..(2): locked nucleic acid guanosine
  modified_base (4)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 90 - DNA, 12 bp, Artificial Sequence
  miR-155 mimic 12926
  misc_feature (1)..(12): joined through phosphorothioate bonds
  modified_base (1)..(1): locked nucleic acid cytidine
  modified_base (3)..(3): locked nucleic acid adenosine
  modified_base (5)..(5): locked nucleic acid thymidine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 91 - DNA, 12 bp, Artificial Sequence
  miR-155 mimic 12927
  misc_feature (1)..(12): joined through phosphorothioate bonds
  modified_base (1)..(1): locked nucleic acid cytidine
  modified_base (4)..(4): locked nucleic acid thymidine
  modified_base (6)..(6): locked nucleic acid adenosine
  modified_base (7)..(7): locked nucleic acid guanosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 92 - DNA, 12 bp, Artificial Sequence
  miR-155 mimic 12928
  misc_feature (1)..(12): joined through phosphorothioate bonds
  modified_base (1)..(1): locked nucleic acid cytidine
  modified_base (4)..(5): locked nucleic acid thymidine
  modified_base (6)..(6): locked nucleic acid adenosine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 93 - DNA, 12 bp, Artificial Sequence
  miR-155 mimic 12929
  misc_feature (1)..(12): joined through phosphorothioate bonds
  modified_base (1)..(1): locked nucleic acid cytidine
  modified_base (4)..(5): locked nucleic acid thymidine
  modified_base (8)..(8): locked nucleic acid cytidine
  modified_base (9)..(9): locked nucleic acid adenosine
  modified_base (10)..(11): locked nucleic acid thymidine
  modified_base (12)..(12): locked nucleic acid adenosine
cgattagcatta

SEQ ID NO: 94 - DNA, 12 bp, Artificial Sequence
  miR-155 mimic 12930
  misc_feature (1)..(12): joined through phosphorothioate bonds
  modified_base (1)..(1): locked nucleic acid cytidine
  modified_base (4)..(5): locked nucleic acid thymidine
  modified_base (7)..(7): locked nucleic acid guanosine
  modified_base (8)..(8): locked nucleic acid cytidine
  modified_base (10)..(11): locked nucleic acid thymidine
  modified_base (12)..(12): locked nucleic acid adenosine
cgattagcatta

SEQ ID NO: 95 - DNA, 11 bp, Artificial Sequence
  miR-155 mimic 12931
  misc_feature (1)..(11): joined through phosphorothioate bonds
  modified_base (3)..(4): locked nucleic acid thymidine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 96 - DNA, 11 bp, Artificial Sequence
  miR-155 mimic 12932
  misc_feature (1)..(11): joined through phosphorothioate bonds
  modified_base (1)..(1): locked nucleic acid guanosine
  modified_base (2)..(2): locked nucleic acid adenosine
  modified_base (3)..(4): locked nucleic acid thymidine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 97 - DNA, 11 bp, Artificial Sequence
  miR-155 mimic 12933
  misc_feature (1)..(11): joined through phosphorothioate bonds
  modified_base (1)..(1): locked nucleic acid guanosine
  modified_base (4)..(4): locked nucleic acid thymidine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 98 - DNA, 11 bp, Artificial Sequence
  miR-155 mimic 12934
  misc_feature (1)..(11): joined through phosphorothioate bonds
  modified_base (1)..(1): locked nucleic acid guanosine
  modified_base (3)..(3): locked nucleic acid thymidine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 99 - DNA, 11 bp, Artificial Sequence
  miR-155 mimic 12935
  misc_feature (1)..(11): joined through phosphorothioate bonds
  modified_base (1)..(1): locked nucleic acid guanosine
  modified_base (3)..(4): locked nucleic acid thymidine
  modified_base (5)..(5): locked nucleic acid adenosine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 100 - DNA, 11 bp, Artificial Sequence
  miR-155 mimic 12936
  misc_feature (1)..(11): joined through phosphorothioate bonds
  modified_base (1)..(1): locked nucleic acid guanosine
  modified_base (3)..(4): locked nucleic acid thymidine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 101 - DNA, 11 bp, Artificial Sequence
  miR-155 mimic 12937
  misc_feature (1)..(11): joined through phosphorothioate bonds
  modified_base (1)..(1): locked nucleic acid guanosine
  modified_base (3)..(4): locked nucleic acid thymidine
  modified_base (6)..(6): locked nucleic acid guanosine
  modified_base (7)..(7): locked nucleic acid cytidine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 102 - DNA, 11 bp, Artificial Sequence
  miR-155 mimic 12938
  misc_feature (1)..(11): joined through phosphorothioate bonds
  modified_base (1)..(1): locked nucleic acid guanosine
  modified_base (3)..(4): locked nucleic acid thymidine
  modified_base (6)..(6): locked nucleic acid guanosine
  modified_base (9)..(10): locked nucleic acid thymidine
  modified_base (11)..(11): locked nucleic acid adenosine
gattagcatta

SEQ ID NO: 103 - DNA, 11 bp, Artificial Sequence
  miR-155 mimic 12939
  misc_feature (1)..(11): joined through phosphorothioate bonds
  modified_base (1)..(1): locked nucleic acid guanosine
  modified_base (3)..(4): locked nucleic acid thymidine
  modified_base (6)..(6): locked nucleic acid guanosine
  modified_base (8)..(8): locked nucleic acid adenosine
  modified_base (10)..(10): locked nucleic acid thymidine
  modified_base (11)..(11): locked nucleic acid adenosine
gattagcatta

SEQ ID NO: 104 - DNA, 11 bp, Artificial Sequence
  miR-155 mimic 12940
  misc_feature (1)..(11): joined through phosphorothioate bonds
  modified_base (1)..(1): locked nucleic acid guanosine
  modified_base (3)..(4): locked nucleic acid thymidine
  modified_base (6)..(6): locked nucleic acid guanosine
  modified_base (8)..(8): locked nucleic acid adenosine
  modified_base (9)..(9): locked nucleic acid thymidine
  modified_base (11)..(11): locked nucleic acid adenosine
gattagcatta

SEQ ID NO: 105 - DNA, 11 bp, Artificial Sequence
  miR-155 mimic 12941
  misc_feature (1)..(11): joined through phosphorothioate bonds
  modified_base (1)..(1): locked nucleic acid guanosine
  modified_base (3)..(4): locked nucleic acid thymidine
  modified_base (6)..(6): locked nucleic acid guanosine
  modified_base (8)..(8): locked nucleic acid adenosine
  modified_base (9)..(10): locked nucleic acid thymidine
gattagcatta

SEQ ID NO: 106 - DNA, 11 bp, Artificial Sequence
  miR-155 mimic 12942
  misc_feature (1)..(11): joined through phosphorothioate bonds
  modified_base (2)..(2): locked nucleic acid adenosine
  modified_base (3)..(4): locked nucleic acid thymidine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 107 - DNA, 11 bp, Artificial Sequence
  miR-155 mimic 12943
  misc_feature (1)..(11): joined through phosphorothioate bonds
  modified_base (1)..(1): locked nucleic acid guanosine
  modified_base (2)..(2): locked nucleic acid adenosine
  modified_base (4)..(4): locked nucleic acid thymidine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 108 - DNA, 11 bp, Artificial Sequence
  miR-155 mimic 12944
  misc_feature (1)..(11): joined through phosphorothioate bonds
  modified_base (1)..(1): locked nucleic acid guanosine
  modified_base (3)..(3): locked nucleic acid thymidine
  modified_base (5)..(5): locked nucleic acid adenosine
  modified_base (6)..(6): locked nucleic acid guanosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 109 - DNA, 11 bp, Artificial Sequence
  miR-155 mimic 12945
  misc_feature (1)..(11): joined through phosphorothioate bonds
  modified_base (1)..(1): locked nucleic acid guanosine
  modified_base (3)..(4): locked nucleic acid thymidine
  modified_base (5)..(5): locked nucleic acid adenosine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 110 - DNA, 11 bp, Artificial Sequence
  miR-155 mimic 12946
  misc_feature (1)..(11): joined through phosphorothioate bonds
  modified_base (1)..(1): locked nucleic acid guanosine
  modified_base (3)..(4): locked nucleic acid thymidine
  modified_base (7)..(7): locked nucleic acid cytidine
  modified_base (8)..(8): locked nucleic acid adenosine
  modified_base (9)..(10): locked nucleic acid thymidine
  modified_base (11)..(11): locked nucleic acid adenosine
gattagcatta

SEQ ID NO: 111 - DNA, 11 bp, Artificial Sequence
  miR-155 mimic 12947
  misc_feature (1)..(11): joined through phosphorothioate bonds
  modified_base (1)..(1): locked nucleic acid guanosine
  modified_base (3)..(4): locked nucleic acid thymidine
  modified_base (6)..(6): locked nucleic acid guanosine
  modified_base (7)..(7): locked nucleic acid cytidine
  modified_base (9)..(10): locked nucleic acid thymidine
  modified_base (11)..(11): locked nucleic acid adenosine
gattagcatta

SEQ ID NO: 112 - DNA, 14 bp, Artificial Sequence
  miR-155 mimic 12954
  misc_feature (1)..(14): joined through phosphorothioate bonds
  modified_base (1)..(1): ethylene-bridged cytidine
  modified_base (3)..(3): ethylene-bridged cytidine
  modified_base (6)..(7): ethylene-bridged thymidine
  modified_base (9)..(9): ethylene-bridged guanosine
  modified_base (11)..(11): ethylene-bridged adenosine
  modified_base (12)..(13): ethylene-bridged thymidine
  modified_base (14)..(14): ethylene-bridged adenosine
cacgattagcatta

SEQ ID NO: 113 - DNA, 14 bp, Artificial Sequence
  miR-155 mimic 12955
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): ethylene-bridged adenosine
  modified_base (12)..(13): locked nucleic acid thymidine
  modified_base (14)..(14): ethylene-bridged adenosine
cacgattagcatta

SEQ ID NO: 114 - DNA, 14 bp, Artificial Sequence
  miR-155 mimic 12956
  misc_feature (1)..(14): joined through phosphorothioate bonds
  modified_base (1)..(1): ethylene-bridged cytidine
  modified_base (3)..(3): ethylene-bridged cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 115 - DNA, 14 bp, Artificial Sequence
  miR-155 mimic 12957
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): ethylene-bridged guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 116 - DNA, 14 bp, Artificial Sequence
  miR-155 mimic 12958
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): ethylene-bridged thymidine
  modified_base (9)..(9): locked nucleic acid guanosine
  modified_base (11)..(11): locked nucleic acid adenosine
  modified_base (12)..(13): ethylene-bridged thymidine
  modified_base (14)..(14): locked nucleic acid adenosine
cacgattagcatta

SEQ ID NO: 117 - DNA, 14 bp, Artificial Sequence
  miR-155 mimic 12959
  misc_feature (1)..(14): joined through phosphorothioate bonds
  modified_base (1)..(1): locked nucleic acid cytidine
  modified_base (3)..(3): locked nucleic acid cytidine
  modified_base (6)..(6): locked nucleic acid thymidine
  modified_base (7)..(7): ethylene-bridged thymidine
  modified_base (9)..(9): locked nucleic acid guanosine
  modified_base (11)..(11): locked nucleic acid adenosine
  modified_base (12)..(13): locked nucleic acid thymidine
  modified_base (14)..(14): locked nucleic acid adenosine
cacgattagcatta

SEQ ID NO: 118 - DNA, 14 bp, Artificial Sequence
  miR-155 mimic 12960
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): locked nucleic acid adenosine
  modified_base (12)..(12): locked nucleic acid thymidine
  modified_base (13)..(13): ethylene-bridged thymidine
  modified_base (14)..(14): locked nucleic acid adenosine
cacgattagcatta

SEQ ID NO: 119 - DNA, 14 bp, Artificial Sequence
  miR-155 mimic 12961
  misc_feature (1)..(14): joined through phosphorothioate bonds
  modified_base (1)..(1): locked nucleic acid cytidine
  modified_base (3)..(3): locked nucleic acid cytidine
  modified_base (6)..(7): locked nucleic acid thymidine
  modified_base (9)..(9): locked nucleic acid guanosine
  modified_base (11)..(11): amino-2′-C-bridged bicyclic adenosine
  modified_base (12)..(13): locked nucleic acid thymidine
  modified_base (14)..(14): amino-2′-C-bridged bicyclic adenosine
cacgattagcatta

SEQ ID NO: 120 - DNA, 14 bp, Artificial Sequence
  miR-155 mimic 12552
  misc_feature (1)..(14): joined through phosphorothioate bonds
  modified_base (1)..(1): amino-2′-C-bridged bicyclic cytidine
  modified_base (3)..(3): amino-2′-C-bridged bicyclic cytidine
  modified_base (6)..(7): amino-2′-C-bridged bicyclic thymidine
  modified_base (9)..(9): amino-2′-C-bridged bicyclic guanosine
  modified_base (11)..(11): amino-2′-C-bridged bicyclic adenosine
  modified_base (12)..(13): amino-2′-C-bridged bicyclic thymidine
  modified_base (14)..(14): amino-2′-C-bridged bicyclic adenosine
cacgattagcatta